(19) United States
(12) Patent Application Publication
(10) Pub. No.: US 2022/0395468 A1
(43) Pub. Date: Dec. 15, 2022

(54) Transdermal Drug Delivery Systems for Administration of a Therapeutically Effective Amount of Lenalidomide and Other Immunomodulatory Agents

(71) Applicant: Starton Therapeutics, Inc., Paramus, NJ (US)

(72) Inventors: Fotios Plakogiannis, Whitestone, NY (US); Rod L. Hartwig, Hamburg, NJ (US); Nisarg Modi, Plainview, NY (US); Tamanna Lather, Livingston, NJ (US); Yuliya Levintova, Brooklyn, NY (US); Marina Borovinskaya, East Brunswick, NJ (US); Arturo Serrano-Batista, Sloatsburg, NY (US)

(21) Appl. No.: 17/831,603

(22) Filed: Jun. 3, 2022

Related U.S. Application Data
(60) Provisional application No. 63/197,427, filed on Jun. 6, 2021.

(51) Int. Cl. A61K 9/70; A61K 31/454; A61K 47/32; A61K 47/20; A61K 47/10; A61K 47/38; A61K 47/12

(52) U.S. Cl. CPC A61K 9/7061; A61K 31/454; A61K 47/32; A61K 47/20; A61K 47/10; A61K 47/38; A61K 47/12; A61K 9/7069

(57) ABSTRACT

Transdermal drug delivery systems and methods of fabricating such systems are provided. The active pharmaceutical ingredient can be lenalidomide or other immunomodulatory agents. More particularly, the present invention is directed to improving the solubility of lenalidomide and other immunomodulatory imide compounds and improving the permeation of such compounds through the skin.

### RELATED APPLICATIONS

The present application claims priority to U.S. Provisional Patent Application No. 63/197,427, filed on Jun. 6, 2021, the entire contents of which are incorporated herein by reference.

### TECHNICAL FIELD

The present disclosure is directed to transdermal drug delivery systems for lenalidomide and other immunomodulatory agents. More particularly, various embodiments are directed to improving the solubility of lenalidomide and other immunomodulatory imide compounds and improving the permeation of such compounds through the skin.

### BACKGROUND OF THE INVENTION

Immunomodulatory imide compounds include thalidomide and thalidomide analogues (collectively the thalidomide family of compounds), which possess pleiotropic anti-myeloma properties including immune-modulation, anti-angiogenic, anti-inflammatory and anti-proliferative effects. The thalidomide analogues include lenalidomide, pomalidomide, and iberdomide.

Lenalidomide (3-(4-amino-1-3-dihydro-1-oxo-2H-isoindol-2yl)-2,6-piperidinedione) or LLD is an FDA approved drug which is available in the form of an oral capsule. Lenalidomide is indicated, for example, for treatment of patients with multiple myeloma (MM) in combination with dexamethasone, MM as maintenance following autologous hematopoietic stem cell transplantation (auto-HSCT), transfusion-dependent anemia due to low- or intermediate-1-risk myelodysplastic syndromes (MDS) associated with a deletion 5q abnormality with or without additional cytogenetic abnormalities, mantle cell lymphoma (MCL) whose disease has relapsed or progressed after two prior therapies, one of which included bortezomib, previously treated follicular lymphoma (FL) in combination with a rituximab product, previously treated marginal zone lymphoma (MZL) in combination with a rituximab product, or chronic lymphocytic leukemia (CLL). Lenalidomide is available in an oral dosing form in strengths of 2.5 mg, 5 mg, 10 mg, 15 mg, 20 mg, and 25 mg.

Pomalidomide (4-amino-2-(2,6-dioxopiperidin-3-yl)isoindoline-1,3-dione is also an FDA approved drug, which is available in the form of oral capsules. Pomalidomide, is typically used, often in combination with dexamethasone, for patients with multiple myeloma who have received prior therapy (such as lenalidomide) and have demonstrated disease progression upon completion (or shortly thereafter) of the last therapy. Pomalidomide is available in an oral dosage form at strengths of 1 mg, 2 mg, 3 mg, and 4 mg.

Thalidomide (2-(2,6-dioxopiperidin-3-yl)isoindole-1,3-dione) is an FDA approved drug, which is available in the form of oral capsules. Thalidomide is typically used, often in combination with dexamethasone, for the treatment of patients with newly diagnosed multiple myeloma. Thalidomide is available in an oral dosage form at strengths of 50 mg, 100 mg, 150 mg, and 200 mg.

Iberdomide ((3S)-3-[7-[[4-(morpholin-4-ylmethyl)phenyl]methoxy]-3-oxo-1H-isoindol-2-yl]piperidine-2,6-dione) is under development for treating refractory multiple myeloma.

Unfortunately, such immunomodulatory imides exhibit very low water solubility. For instance, lenalidomide (LLD) purportedly exhibits very slight water solubility, as provided for in prescribing information for Revlimid, which states: “Solubility was significantly lower in less acidic buffers, ranging from about 0.4 to 0.5 mg/m L.” Recent work has shown that the drug has very limited solubility in water-based solutions and in most of the common pharmaceutically accepted organic solvents.

The currently approved drug product is a solid oral dosage form presented as powder filled capsules. Thus, the drug is maintained in a solid state. The solid state of the drug exhibits very good stability with its current dosage formulation as Revlimid is offered with a room temperature shelf life of 24 months from Date of Manufacture.

However, such oral administration results in a cycle of high and low drug levels caused by oral administration, which are associated with unpleasant and often debilitating side effects. For example, 40% of people with high-risk SMM treated with oral lenalidomide discontinued due to drug-related side effects, despite 91% achieving three-year progression-free survival versus the 66% who were monitored without receiving active treatment (the current standard of care).

Meanwhile, transdermal drug delivery systems are typically available in a solubilized drug-in-adhesive formulation in their simplest formulations. With drugs exhibiting challenging solubility and permeability requirements, modifications to the formulation are needed to maintain the drug in solution and to provide for alternative pathways to solubility upon application and/or specific permeability enhancers are required to increase the permeability of the drug molecules. For instance, transdermal formulations for lenalidomide are challenging due to the low solubility of the LLD in solution of less than 0.4-0.5 mg/mL based on LLD's high melting point of around 270° C. and its highly crystalline nature. Further, LLD's low log P value of −0.4 is indicative of the difficulty of the drug molecule to penetrate the stratum corneum.

In consideration of the aforementioned problems, a need exists for a transdermal drug delivery system where the solubility of immunomodulatory drugs including lenalidomide (LLD) is improved and where the delivery of the drug through the skin is improved. A need also exists for immunomodulatory drugs that can be delivered in a controlled release format for a long period of time. Still another need exist for reducing the side effects associated with high oral doses of immunomodulatory drugs.

### SUMMARY OF THE INVENTION

In accordance with one embodiment of the present invention, a transdermal drug delivery system is disclosed. The transdermal drug delivery system includes a solubilized drug in adhesive layer including an active pharmaceutical ingredient comprising an immunomodulatory agent, a pressure sensitive adhesive, a crystallization inhibitor, and optionally a polar aprotic solvent, wherein the immunomodulatory agent is homogeneously dissolved in the solubilized drug-in-adhesive layer and is present in an amount ranging from about 0.1 wt. % to about 50 wt. % based on the dry weight of the solubilized drug in adhesive layer. Moreover, the transdermal drug delivery system is a single, double, or multi-layered structure.

In one aspect, the immunomodulatory agent includes lenalidomide, pomalidomide, iberdomide or thalidomide. Moreover, in an aspect, the pressure sensitive adhesive is an acrylate copolymer, a polyisobutylene, a silicone, or a combination thereof. In a further aspect, the pressure sensitive adhesive is the acrylate copolymer. In yet another aspect, the crystallization inhibitor is a polyvinylpyrrolidone.

Additionally or alternatively, in an aspect, the transdermal drug delivery system also includes a thickener. In one aspect, the thickener is cellulose, a cellulose derivative, methylcellulose, ethyl cellulose, carboxymethyl cellulose, hydroxylpropyl cellulose, hydroxylpropylmethyl cellulose, hydroxypropyl methylcellulose, acrylate, an acrylate derivative, or a combination thereof.

In yet another aspect, the transdermal drug delivery system includes a skin permeation enhancer. In one aspect, the skin permeation enhancer includes a fatty acid or one of its derivatives, a fatty alcohol or one of its derivatives, a fatty ester or one of its derivatives, a surfactant, a solubilizer, a plasticizer, an emollient, a skin irritation-reducing agent, a buffering agent, or a combination thereof.

Moreover, in an aspect, the transdermal drug delivery system also includes a skin modifier that can be a butylated hydroxytoluene (BHT), butylated hydroxyanisole (BHA), gallic acid, ascorbic acid, ascorbyl palmitate, lactic acid, methyl salicylate, salicylic acid, or a combination thereof and/or a polar aprotic solvent that can be a n-methyl-2-pyrrolidone (NMP), dimethyl sulfoxide (DMSO), dimethylformamide, dimethyl isosorbide, or a combination thereof.

In an aspect, the transdermal drug delivery system can also include a backing layer and a release liner, where the backing layer forms an exterior facing-surface of the transdermal drug delivery system and the release liner is positioned adjacent a skin contacting surface of the solubilized drug in adhesive layer, where the solubilized drug in adhesive layer forms 0.1 wt. % to about 50 wt. % of the transdermal drug delivery system.

In accordance with another embodiment of the present invention, a transdermal drug delivery system is disclosed. The transdermal drug delivery system includes a solid dispersion of a drug in adhesive layer including an active pharmaceutical ingredient comprising an immunomodulatory agent, a pressure sensitive adhesive, a crosslinked polyvinylpyrrolidone, and a skin permeation enhancer comprising a surfactant, wherein the immunomodulatory agent is homogeneously dispersed throughout the solid dispersion of the drug in adhesive layer and is present in an amount ranging from about 0.1 wt. % to about 25 wt. % based on the dry weight of the solid dispersion of the drug in adhesive layer. Further, the surfactant can include at least one non-ionic surfactant, which may be used in combination with one or more humectants, permeation enhancers, solubilizers, plasticizers, or a combination thereof. Moreover, the transdermal drug delivery system is a single, double, or multi-layered structure.

In one aspect, the immunomodulatory agent includes lenalidomide, pomalidomide, iberdomide or thalidomide. Moreover, in an aspect, the pressure sensitive adhesive is an acrylate copolymer, a polyisobutylene, a silicone, or a combination thereof. In a further aspect, the pressure sensitive adhesive is the acrylate copolymer. In yet another aspect, the crystallization inhibitor is a polyvinylpyrrolidone.

Furthermore, in an aspect, the crosslinked polyvinylpyrrolidone is present in the solid dispersion drug in adhesive layer in an amount ranging from about 0.1 wt. % to about 40 wt. % based on the dry weight of the solid dispersion drug in adhesive. In yet another aspect, a ratio of the immunomodulatory agent to the crosslinked polyvinylpyrrolidone is from about 1:10 to about 4:1.

Additionally or alternatively, in one aspect, the transdermal drug delivery system also includes a dispersing agent, where the dispersing agent can be a mineral oil, a silicone fluid, a fatty acid ester, or a combination thereof. In one aspect, the skin permeation enhancer also includes a fatty acid or one of its derivatives, a fatty alcohol or one of its derivatives, a fatty ester or one of its derivatives, a solubilizer, a plasticizer, an emollient, a skin irritation-reducing agent, a buffering agent, an antioxidant a preservative, or a combination thereof.

Moreover, in an aspect, the surfactant is a non-ionic surfactant, such as a polyoxyethylene or polyethylene glycol ether of a fatty derivative which comprises an oleic acid or oleyl alcohol derivative, a lauric acid or lauryl alcohol derivative, cetyl or ceteryl alcohol, stearic acid or stearyl alcohol or similar fatty derivative of polyoxyethylene, a poloxamer, or a combination thereof.

In yet another aspect, the transdermal drug delivery system also includes a skin or adhesive modifier that can be butylated hydroxytoluene (BHT), butylated hydroxyanisole (BHA), gallic acid, ascorbic acid, ascorbyl palmitate, lactic acid, methyl salicylate, salicylic acid, or a combination thereof. Furthermore, in one aspect, the polar aprotic solvent is n-methyl-2-pyrrolidone (NMP), dimethyl sulfoxide (DMSO), dimethylformamide, or a combination thereof.

In an aspect, the transdermal drug delivery system can also include a backing layer and a release liner, where the backing layer forms an exterior facing-surface of the transdermal drug delivery system and the release liner is positioned adjacent a skin contacting surface of the solubilized drug in adhesive layer, where the solubilized drug in adhesive layer forms 0.1 wt. % to about 50 wt. % of the transdermal drug delivery system.

In accordance with yet another embodiment of the present invention, a transdermal drug delivery system is disclosed. The transdermal drug delivery system includes a non-drug containing adhesive layer including a pressure sensitive adhesive; and a drug containing polymer layer comprising an immunomodulatory agent, a crystallization inhibitor, and optionally a polar aprotic solvent, wherein the immunomodulatory agent is homogeneously dissolved and/or dispersed in the drug containing polymer layer and is present in an amount ranging from about 0.1 wt. % to about 50 wt. % based on the dry weight of the drug containing polymer layer. Moreover, the transdermal drug delivery system is a single, double, or multi-layered structure.

In one aspect, the pressure sensitive adhesive is an acrylate copolymer, a polyisobutylene, a silicone, or a combination thereof. In a further aspect, the pressure sensitive adhesive is the acrylate copolymer, where the immunomodulatory agent has a solubility of less than about 0.5 mg/mL in the acrylate copolymer. In yet another aspect, the crystallization inhibitor is a polyvinylpyrrolidone. Moreover, in an aspect, the immunomodulatory agent is lenalidomide.

Additionally or alternatively, in an aspect, the transdermal drug delivery system also includes a thickener. In one aspect, the thickener is cellulose, a cellulose derivative, methylcellulose, ethyl cellulose, carboxymethyl cellulose, hydroxylpropyl cellulose, hydroxylpropylmethyl cellulose, hydroxypropyl methylcellulose, acrylate, an acrylate derivative, or a combination thereof.

In yet another aspect, the transdermal drug delivery system includes a skin permeation enhancer. In one aspect, the skin permeation enhancer includes a fatty acid or one of its derivatives, a fatty alcohol or one of its derivatives, a fatty ester or one of its derivatives, a surfactant, a solubilizer, a plasticizer, an emollient, a skin irritation-reducing agent, a buffering agent, or a combination thereof.

Moreover, in an aspect, the transdermal drug delivery system also includes a skin modifier that can be a butylated hydroxytoluene (BHT), butylated hydroxyanisole (BHA), gallic acid, ascorbic acid, ascorbyl palmitate, lactic acid, methyl salicylate, salicylic acid, or a combination thereof Furthermore, in one aspect, the polar aprotic solvent is a n-methyl-2-pyrrolidone (NMP), dimethyl sulfoxide (DMSO), dimethylformamide, dimethyl isosorbide, or a combination thereof.

In an aspect, the transdermal drug delivery system can also include a backing layer and a release liner, where the backing layer forms an exterior facing-surface of the transdermal drug delivery system and the release liner is positioned adjacent a skin contacting surface of the solubilized drug in adhesive layer, where the solubilized drug in adhesive layer forms 0.1 wt. % to about 50 wt. % of the transdermal drug delivery system.

In accordance with still another embodiment of the present invention a pretreatment composition for enhancing permeation of an immunomodulatory agent through a patient's skin is disclosed. The pretreatment composition includes a polar aprotic solvent; a humectant; an organic acid; and a thickener.

In one aspect, the polar aprotic solvent is n-methyl-2-pyrrolidone (NMP), dimethyl sulfoxide (DMSO), dimethylformamide, or a combination thereof. In yet another aspect, the humectant is glycerin, glycol, a glycol derivative, polyglycol, polyethylene glycol, triethyl citrate, triacetin, a surfactant, a permeability enhancer, or a combination thereof. Additionally or alternatively, in an aspect, the organic acid is levulinic acid, oleic acid, lactic acid, salicylic acid, or a combination thereof. In yet a further aspect, the thickener is cellulose, a cellulose derivative, methylcellulose, ethyl cellulose, carboxymethyl cellulose, hydroxylpropyl cellulose, hydroxylpropylmethyl cellulose, hydroxypropyl methylcellulose, acrylate, an acrylate derivative, or a combination thereof. Moreover, in an aspect, the patient's skin is in contact with the pretreatment composition for a time period ranging from about 1 minute to about 72 hours.

The present disclosure is also generally directed to a kit that includes a transdermal drug delivery system and a pretreatment composition according to any one or more of the above aspects. In one aspect, the patient's skin is in contact with the pretreatment composition for a time period ranging from about 1 minute to about 72 hours.

In accordance with one embodiment of the present invention, a transdermal drug delivery system is disclosed. The transdermal drug delivery system includes a solubilized drug in adhesive layer including an active pharmaceutical ingredient comprising an immunomodulatory agent, a pressure sensitive adhesive, a crystallization inhibitor, and optionally a polar aprotic solvent, wherein the immunomodulatory agent is homogeneously dissolved in the solubilized drug-in-adhesive layer and is present in an amount ranging from about 0.1 wt. % to about 50 wt. % based on the dry weight of the solubilized drug in adhesive layer. Moreover, the transdermal drug delivery system is a single, double, or multi-layered structure, where a pretreatment composition according to any one or more of the above aspects is disposed adjacent to the solubilized drug in adhesive layer.

In yet another aspect, a patient's skin is in contact with the pretreatment composition for a time period ranging from about 1 minute to about 72 hours prior to the patient's skin coming into contact with the solubilized drug in adhesive layer.

In accordance with another embodiment of the present invention, a transdermal drug delivery system is disclosed. The transdermal drug delivery system includes a solid dispersion of a drug in adhesive layer including an active pharmaceutical ingredient comprising an immunomodulatory agent, a pressure sensitive adhesive, a crosslinked polyvinylpyrrolidone, and a skin permeation enhancer comprising a surfactant, wherein the immunomodulatory agent is homogeneously dispersed throughout the solid dispersion of the drug in adhesive layer and is present in an amount ranging from about 0.1 wt. % to about 25 wt. % based on the dry weight of the solid dispersion of the drug in adhesive layer. Further, the surfactant can include at least one non-ionic surfactant, which may be used in combination with one or more humectants, permeation enhancers, solubilizers, plasticizers, or a combination thereof. Moreover, the transdermal drug delivery system is a single, double, or multi-layered structure, where a pretreatment composition according to any one or more of the above aspects is disposed adjacent to the solid dispersion drug in adhesive layer.

In one such aspect, a patient's skin is in contact with the pretreatment composition for a time period ranging from about 1 minute to about 72 hours prior to the patient's skin coming into contact with the solubilized drug in adhesive layer.

In accordance with yet another embodiment of the present invention, a transdermal drug delivery system is disclosed. The transdermal drug delivery system includes a non-drug containing adhesive layer including a pressure sensitive adhesive; and a drug containing polymer layer comprising an immunomodulatory agent, a crystallization inhibitor, and a polar aprotic solvent, wherein the immunomodulatory agent is homogeneously dissolved and/or dispersed in the drug containing polymer layer and is present in an amount ranging from about 0.1 wt. % to about 50 wt. % based on the dry weight of the drug containing polymer layer. Moreover, the transdermal drug delivery system is a single, double, or multi-layered structure, where a pretreatment composition according to any one or more of the above aspects is disposed adjacent to the solubilized drug in adhesive layer.

In yet another aspect, a patient's skin is in contact with the pretreatment composition for a time period ranging from about 1 minute to about 72 hours prior to the patient's skin coming into contact with the solubilized drug in adhesive layer.

Additionally or alternatively, the immunomodulatory agent of any one or more of the above aspects treats chronic lymphocytic leukemia or multiple myeloma. Furthermore, in an aspect, the immunomodulatory agent of any one or more of the above aspects is provided in the transdermal drug delivery system in a therapeutic concentration for the treatment of leukemia or multiple myeloma. In yet another aspect, immunomodulatory agent of any one or more of the above aspects is delivered through the skin such that a plasma concentration of from about 1 nanogram/milliliter to about 100 nanograms per milliliter is achieved. Moreover, in one aspect, the transdermal delivery system of any one or more of the above aspects provides continuous delivery of the immunomodulatory agent for a time period ranging from about 1 day to about 15 days.

In accordance with yet another embodiment of the present invention, a transdermal drug delivery system is disclosed. The transdermal drug delivery system includes an immunomodulatory agent and a material for delivering the immunomodulatory agent through a patient's skin, wherein the material comprises a topical formulation, a gel, a lotion, a spray, a metered dose transdermal spray, an aerosol, a suppository, a magma, a transdermal patches, a bilayer matrix patch, a multilayer matrix patch, a monolithic matrix patch with or without adhesive, a drug in adhesive patch, a matrix reservoir patch, a microreservoir patch, a hydrogel matrix patch, a mucoadhesive patch, an adhesive system, a transdermally applicable tape, a microneedle, or an iontophoresis system.

Other features and aspects of the present invention are set forth in greater detail below.

### BRIEF DESCRIPTION OF THE DRAWINGS

A full and enabling disclosure of the present invention, including the best mode thereof to one skilled in the art, is set forth more particularly in the remainder of the specification, including reference to the accompanying figure, in which:

FIG. 1 is a cross-sectional view of a transdermal drug delivery system according to one embodiment of the present disclosure, where the transdermal drug delivery system includes a stable, solubilized drug in adhesive formulation;

FIG. 2 is a cross-sectional view of a transdermal drug delivery system according to another embodiment of the present disclosure, where the transdermal drug delivery system includes a stable solid drug dispersion in adhesive;

FIG. 3 is a cross-sectional view of a transdermal drug delivery system according to another embodiment of the present disclosure; where the transdermal drug delivery system includes a multilayer adhesive-polymer matrix formulation to provide controlled release of the drug from the polymer matrix;

FIG. 4A is a cross-sectional view of a kit containing a pretreatment composition and one of the transdermal drug delivery systems of FIGS. 1-3;

FIG. 4B is a cross-sectional view of a of a transdermal drug delivery system according to one embodiment of the present disclosure, where the transdermal drug delivery system includes a stable drug in adhesive formulation and a pretreatment solution;

FIG. 4C is a cross-sectional view of a transdermal drug delivery system according to another embodiment of the present disclosure, where the transdermal drug delivery system includes a stable solid drug dispersion in adhesive and a pretreatment solution;

FIG. 4D is a cross-sectional view of a transdermal drug delivery system according to another embodiment of the present disclosure, where the transdermal drug delivery system includes a multilayer adhesive-polymer matrix formulation and a pretreatment solution to provide controlled release of the drug from the polymer matrix;

FIG. 5 is a flow chart illustrating a method of making the transdermal drug delivery system of FIG. 1;

FIG. 6 is a flow chart illustrating a method of making the transdermal drug delivery system of FIG. 2;

FIG. 7 is a flow chart illustrating a method of making the transdermal drug delivery system of FIG. 3;

FIG. 8 is a flow chart illustrating a method of using the kit including a transdermal drug delivery system of FIG. 4A;

FIG. 9 is a flow chart illustrating a method of making the transdermal drug delivery systems of FIGS. 4B-4D;

FIG. 10 is a graph describing the level of lenalidomide permeation through human cadaver skin for solutions of lenalidomide formed with various solvents;

FIG. 11 is a graph describing the level of lenalidomide permeation through human cadaver skin for gels of lenalidomide formed with various permeation enhancers;

FIG. 12 is another graph describing the level of lenalidomide permeation through human cadaver skin for gels of lenalidomide formed with various permeation enhancers;

FIG. 13 is a graph describing the level of lenalidomide permeation through human cadaver skin for various drug in adhesive matrix patch formulations after a pretreatment gel formulation was applied prior to applying the patch to the skin;

FIG. 14 is a graph describing the flux (micrograms/square centimeter/hour) of lenalidomide through human cadaver skin for various stable solid drug dispersion in adhesive formulations;

FIG. 15 is a graph describing the cumulative flux of lenalidomide (micrograms/square centimeter) through human cadaver skin for various stable solid drug dispersion in adhesive formulations;

FIG. 16 is a graph describing the flux of lenalidomide through human cadaver skin after pretreating the skin with a pretreatment composition according to one embodiment of the present disclosure;

FIG. 17 is a graph describing the effect of various concentrations of salicylic acid pretreatment gels on the flux of lenalidomide through human cadaver skin from a drug in adhesive matrix patch;

FIG. 18 is a graph describing the effect of various pretreatment gel compositions on the flux of lenalidomide from a drug in adhesive matrix patch; and

FIG. 19 is a graph describing the flux of lenalidomide through human cadaver skin from five drug in adhesive matrix and drug in polymer patches after one hour.

FIG. 20 is a graph comparing the average cumulative area under the curve (AUC) for four formulations of lenalidomide delivered via various transdermal drug delivery systems over a time period of 168 hours in a rabbit model. Group 2 was in the form of a solid dispersion of a drug in adhesive layer; Group 3 was in the form of a solid dispersion of a drug in adhesive layer that was applied after a DMSO pretreatment; Group 4 was in the form of an adhesive matrix that was applied after a formulated pretreatment; and Group 5 was in the form of a polymer film applied after a formulated pretreatment. As can be seen from FIG. 20, both formulated pretreatments exhibited a characteristic oral or IV administration delivery profile. Meanwhile, the solid dispersion of the drug in adhesive layer without a pretreatment and with a DMSO pretreatment exhibited a sustained near first-order delivery profile suggesting longer delivery profiles are possible up to 3-days utilizing the transdermal drug delivery systems contemplated by the present invention.

FIG. 21 is a graph comparing the average flux for the four formulations described in FIG. 20 over a time period of 72 hours.

FIG. 22 is a graph showing the permeation of lenalidomide through a Strat-M membrane using a control formulation which was shown to be the only formula to permeate.

FIG. 23 is a graph showing the improved permeation of lenalidomide through a Strat-M membrane that included a non-ionic surfactant containing both an oleth-based surfactant and a poloxamer-based surfactant, demonstrating that permeation is improved with an oleth and a poloxamer compared to an oleth alone. As shown, there is a significant increase in AUC due to an increase in the available lenalidomide as well as a permeability improvement, where it is believed that the poloxamer (e.g., P407) improves the solubility of the lenalidomide in the presence of water and the oleth improves the permeation of the available lenalidomide. This demonstrates that the inclusion of a poloxamer (specifically P407) can lead to significant improvement in the solubility of the lenalidomide in the presence of water and that the oleth can contribute to the permeation of the available lenalidomide.

Repeat use of reference characters in the present specification and drawing is intended to represent the same or analogous features or elements of the present invention.

### DETAILED DESCRIPTION OF REPRESENTATIVE EMBODIMENTS

It is to be understood by one of ordinary skill in the art that the present discussion is a description of exemplary embodiments only, and is not intended as limiting the broader aspects of the present invention.

Generally, the present invention is directed to a drug in adhesive patch placed on the skin to deliver immunomodulatory agents such as lenalidomide continuously at a lower dose than that in approved oral formulations. The formulations of the present invention enhance an immunomodulatory agent's solubility and stability and enable people to avoid the cycle of high and low drug levels caused by oral administration, which is expected to increase efficacy with fewer side effects.

The formulations of the present invention have potential in expanding the standard of care for myeloma treatment. For example, 40% of people with high-risk SMM treated with oral lenalidomide discontinued due to drug-related side effects, despite 91% achieving three-year progression-free survival versus the 66% who were monitored without receiving active treatment (the current standard of care). The formulations contemplated by the present invention can also be used to treat will be the first maintenance therapies to help people stay in remission, where dose-related side effects previously limited the success of oral lenalidomide formulations despite showing efficacy in CLL.

Transdermal Drug Delivery System with Solubilized Drug in Adhesive Layer

In one embodiment, the present invention is directed to a transdermal drug delivery system for the delivery of an immunomodulatory agent through the skin. In one particular embodiment, the immunomodulatory agent can be lenalidomide, although it is to be understood that in alternative embodiments, any other immunomodulatory agent can be utilized in the transdermal drug delivery system. The transdermal drug delivery system includes a solubilized drug in adhesive layer that includes the immunomodulatory agent (e.g., lenalidomide), a pressure sensitive adhesive, and a solubilizing agent or a crystallization inhibitor. The transdermal drug delivery system may also include a plasticizer or humectant that serves as a skin permeation enhancer, a thickener, a skin and/or adhesive modifier such as a filler, a protectant, an antioxidant, an excipient that improves the release of the immunomodulatory agent, or a combination thereof. Further, the solubilized drug in adhesive layer can utilize one or more polar aprotic solvents to ensure that the immunomodulatory agent is solubilized and homogeneously distributed within the solubilized drug in adhesive layer. When utilized as a process solvent, the one or more polar aprotic solvents can be detectable in transdermal drug delivery system in an amount less than about 530 parts per million, or less than about 0.053 wt. %, while when utilized as an excipient, the one or more polar aprotic solvents can be present in an amount greater than about 530 parts per million, or greater than about 0.053 wt. %. Without intending to be limited by any particular theory, the present inventors have found that the specific components of the solubilized drug in adhesive layer and the method by which the immunomodulatory agent is solubilized in the drug in adhesive layer improves its solubility in the blend and enhances its permeation through the skin.

Referring to FIG. 1 and according to one particular embodiment, the transdermal drug delivery system 100 includes a solubilized drug in adhesive layer 110 disposed between a backing layer 120 and a release liner 130. The backing layer 120 has an exterior surface 140 that is exposed to the ambient environment when the transdermal drug delivery system 100 is in use. Meanwhile, the release liner 130 is positioned on a skin-contacting surface 150 of the solubilized drug in adhesive matrix layer 110, where the release liner 130 is removable so that the drug in adhesive layer 110 can be positioned directly on the skin during use of the transdermal drug delivery system 100. As a result of the specific combination of components in the solubilized drug in adhesive layer, such as the particular polar aprotic solvents and solubilization agent or crystallization inhibitors, as well as the specific weight percentages and ratios of such components utilized, the present inventors have found that the transdermal drug delivery system 100 can include a solubilized and homogeneous drug-in-adhesive matrix layer that forms a skin-contacting surface, which facilitates the delivery of the immunomodulatory agent (i.e., the active pharmaceutical ingredient or API) in a controlled manner. For instance, the solubility of the immunomodulatory agent can be improved to at least 2 milligrams per milliliter (mg/mL) or 2 wt. %, such as about 4 mg/mL or 4 wt. % up to about 70 mg/mL or 7 wt. %, which is at least about an 8-fold increase up to about a 140 fold increase over the known solubility of lenalidomide in aqueous buffers (e.g., in a 1:1 solution of DMF:PBS at pH of 7.2). As shown in FIG. 1, the solubilized drug in adhesive layer 110 can be in the form of a single layer so that the active pharmaceutical ingredient is homogeneously dispersed throughout adhesive component of the device 100. However, it should also be understood that additional drug in adhesive layers may also be included in the transdermal drug delivery system 100.

The various components of the transdermal drug delivery system 100 are discussed in detail below.

### I. Solubilized Drug in Adhesive Layer

a. Active Pharmaceutical Ingredient

The polymer blend use to form the drug in adhesive layer of the transdermal drug delivery system of the present invention can include any suitable drug or active pharmaceutical ingredient (API) that functions as an immunomodulatory agent. For instance, the immunomodulatory agent can include all pharmaceutically acceptable forms of an immunomodulatory imide compound, such as thalidomide, including analogs of thalidomide including lenalidomide, pomalidomide, and iberdomide including, for example, free base, salts, polymorphs, solvates, solutions, isomers, amorphous, crystalline, co crystalline, solid solution, prodrugs, analogs, derivatives, and metabolites and combinations thereof. The compound may be in the form of a pharmaceutically acceptable salt, such as an acid addition salt or a base salt, or a solvate thereof, including a hydrate thereof. Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methane sulphonate, ethanesulphonate, benzenesulphonate, p-toluenesulphonate and pamoate salts.

Regardless of the particular immunomodulatory agent utilized as the API, the amount of the API contained in the solubilized drug in adhesive layer can range from about 0.1 wt. % to about 50 wt. %, such as from about 0.5 wt. % to about 25 wt. %, such as from about 0.75 wt. % to about 10 wt. % based on the dry weight of the solubilized drug in adhesive layer. Further, it is to be understood that the immunomodulatory agent is homogeneously dissolved in the solubilized drug in adhesive layer despite being present at such high concentrations in the drug in adhesive layer.

b. Pressure Sensitive Adhesive

The solubilized drug in adhesive layer of the transdermal drug delivery system of the present invention includes one or more suitable pressure sensitive adhesive (PSA). Adhesive polymers may be made from various materials which include plastics, polymers, pressure sensitive adhesives, self-adhering systems, or may require additional excipients to obtain pressure sensitive properties. Basic adhesive systems are selected from polyacrylics, silicones, polyisobutylenes, rubbers, and combinations thereof either by physical blending or copolymerization is disclosed. These materials may be obtained from solvent-borne, water-borne, physical mixtures, extruded, co-extruded, hot melt, or otherwise formed as polymerized or unpolymerized materials.

In one embodiment, the PSA can be an acrylic polymer. Useful acrylic polymers include various homopolymers, copolymers, terpolymers and the like of acrylic acids and derivatives thereof as a cross-linked, cross-linkable, uncross-linked, uncross-linkable, grafted, block, cured and non-curing pressure sensitive adhesives (PSAs). These acrylic polymers include copolymers of alkyl acrylates or methacrylates. Polyacrylates include acrylic acid, methacrylic acid, and derivatives thereof without limitation, methyl acrylate, methyl methacrylate, ethyl acrylate, ethyl methacrylate, n-butyl acrylate, n-butyl methacrylate, hexyl acrylate, 2-ethylbutyl acrylate, isooctyl acrylate, 2-ethylhexyl acrylate, 2-ethylhexyl methacrylate, decyl acrylate, decylmethacrylate, dodecyl acrylate, dodecyl methacrylate, tridecyl acrylate, tridecyl methacrylate, vinyl acetate, 2-hydroxyethyl acrylate, glycidyl methacrylate, or octylacrylamide. The acrylic polymer may be functional species with levels of hydroxyl or carboxyl moieties or combinations thereof, non-functional species without functional moieties, non-reactive species with moieties which are less reactive than hydroxyl or carboxyl moieties, such as methyl or ethyl or propyl or butyl capped acrylamides. Exemplary acrylic PSAs include, without limitation, one or more of: Duro-Tak® 87-900A, Duro-Tak 87-9301 (36.5% solids content acrylate polymer with no functional groups and having a viscosity of about 9500 centipoise), Duro-Tak® 87-4098, Duro-Tak® 387-2510/87-2510, Duro-Tak® 387-2287/87-2287, Duro-Tak® 87-4287, Duro-Tak® 387-2516/87-2516, Duro-Tak® 87-2074, Duro-Tak® 87-235A, Duro-Tak 387-2353/87-2353, Gelva® GMS 9073, Duro-Tak® 87-2852, Duro-Tak® 387-2051/87-2051, Duro-Tak® 387-2052/87-2052, Duro-Tak® 387-2054/87-2054, Duro-Tak® 87-2194, or Duro-Tak® 87-2196. It should also be understood that the disclosure herewith incorporates known and unknown naming conventions comprising the monomers disclosed.

In one particular embodiment, the present inventors have found that the use of a PSA that includes an acrylate copolymer having —COOH or —OH functional groups contributes to the improved solubility of the immunomodulatory agent contained in the drug in adhesive layer. Further, it has also been found that an acrylate copolymer having a solids content ranging from about 30% to about 55%, such as from about 35% to about 50%, such as from about 36% to about 45% also contributes to the improved solubility of the immunomodulatory agent. Additionally, an acrylate copolymer having a viscosity of less than about 6500 centipoise, such as from about 2000 centipoise to about 5000 centipoise, such as from about 2500 centipoise to about 4500 centipoise may also contribute to the improved solubility of the immunomodulatory agent, where the viscosity impacts the loading capacity of the components in polymer blend used to form the drug in adhesive matrix layer. Further, an acrylate copolymer that includes vinyl acetate may also be beneficial. Particular examples include Duro-Tak® 387-2516/87-2516 (vinyl acetate; —OH functional groups; 41.5% solids content; viscosity of 4350 centipoise), Duro-Tak® 387-2052/87-2052 (vinyl acetate; —COOH functional groups, 47.5% solids content; viscosity of 2750 centipoise), or Duro-Tak® 87-4098 (vinyl acetate; 38.5% solids content with no functional groups; viscosity of 6500 centipoise).

In one particular embodiment, the present inventors have found that the use of a PSA that includes an acrylate copolymer having no functional groups in the drug in adhesive layer particularly beneficial. Such PSAs include Duro-Tak® 87-9301 and Duro-Tak® 87-4098.

In still another embodiment, the PSA can include silicone. Suitable silicone adhesives include pressure sensitive adhesives made from silicone polymer and resin. The polymer to resin ratio can be varied to achieve different levels of tack. Specific examples of useful silicone adhesive which are commercially available include the standard BIO-PSA® series (7-4400, 7-4500, and 7-4600 series) and the amine compatible (end capped) BIO-PSA® series (7-4100, 7-4200, and 7-4300 series) manufactured by Dow Corning. Preferred adhesives include BIO-PSA® 7-4101, 7-4102, 7-4201, 7-4202, 7-4301, 7-4302, 7-4401, 7-4402, 7-4501, 7-4502, 7-4601, and 7-4602, where PSAs ending in 1 comprise Heptane as process solvent and PSAs ending in 2 comprise ethyl acetate as the process solvent.

In still another embodiment, the PSA can include polyisobutylene. Suitable polyisobutylene adhesives are those which are pressure sensitive and have suitable tack. The polyisobutylene can comprise a mixture of high and medium molecular weight polyisobutylenes, polybutenes, and mineral oils. Specifically, high molecular weight polyisobutylenes are those with a molecular weight of at least about 425,000. Medium molecular weight polyisobutylenes are those with a molecular weight of at least 40,000 but less than about 425,000. Low molecular weight polyisobutylenes are those with a molecular weight of at least 100 but less than about 40,000. Specific examples of useful polyisobutylene adhesives which are commercially available include Oppanol® High Molecular Weight N grades 50, 50SF, 80, 100 and 150, and Oppanol® Medium Molecular Weight B grades 10N, 10SFN, 11SFN, 12SFN, 12N, 13SFN, 14SFN, 15SFN, and 15N manufactured by BASF. Specific examples of polybutenes are commercially available from Soltex as polybutenes of various molecular weights and by Ineos as Indopol and Panalane with various molecular weights. A specific example of a useful polyisobutylene adhesive which is commercially available includes Duro-Tak® 87-6908.

Other pressure sensitive adhesives obtained from rubber block copolymers, such as Styrene-Isoprene-Styrene (SIS) or Styrene-Butadiene-Styrene (SBS, based adhesives are also contemplated by the present invention.

Regardless of the particular PSA utilized, the pressure sensitive adhesive can be present in an amount ranging from about 1 wt. % to about 99 wt. %, such as from about 20 wt. % to about 98.5 wt. %, such as from about 40 wt. % to about 98 wt. % based on the dry weight of the solubilized drug in adhesive layer.

c. Solubilization Agent/Crystallization Inhibitor

The solubilized drug in adhesive layer of the transdermal drug delivery system of the present invention can also include one or more solubilization agents or crystallization inhibitors that can include polyvinylpyrrolidone (PVP), such as uncrosslinked PVP. Without intending to be limited by any particular theory, the present inventors have found that the uncrosslinked PVP may function in a polar aprotic nature by structure, the polymer contains a 5-member ring with a tertiary amine and a ketone in a specific arrangement. Thereby, this type of polymer avoids the use of an alcohol (—OH) group excipient, yet provides for a structure which is polar aprotic in nature. Suitable soluble grades of PVP as provided by BASF can include Kollidon® grades K-12 (molecular weight range 2,000-3,000; pH 4.63), K-17 (molecular weight 7,000-11,000; pH 4.64), K-25 (molecular weight 28,000-34,000; pH 4.00), K-30 (molecular weight 44,000-54,000; pH 4.10), and K-90 (molecular weight 1,000,000-1,500,000; pH 5.68. Other functional polymers may include Kollidon® VA64 (molecular weight range 45,000-70,000, pH 4.51) or other povidones and copolymers thereof by different vendors. The present inventors have found that the use of polyvinylpyrrolidone in the presence of lenalidomide increases the solubility and stability of the lenalidomide. Also, it is to be understood that acids such as but not limited to lactic acid and levulinic acid can function as lenalidomide solubilizing agents.

The amount of the polyvinylpyrrolidone contained in the solubilized drug in adhesive layer can range from about 0.5 wt. % to about 50 wt. %, such as from about 0.75 wt. % to about 25 wt. %, such as from about 1 wt. % to about 15 wt. % based on the dry weight of the solubilized drug in adhesive layer.

d. Thickener

The solubilized drug in adhesive layer of the transdermal drug delivery system of the present invention can also include one or more thickening agents. The one or more thickening agents can include natural polymers, polysaccharides and their derivatives such as but not limited to agar, alginic acid and derivatives, cassia tora, collagen, gelatin, gellum gum, guar gum, pectin, potassium, or sodium carageenan, tragacanth, xantham, gum copal, chitosan, resin etc., semisynthetic polymers and its derivatives such as without any limitation to cellulose and its derivatives (methylcellulose, ethyl cellulose, carboxymethyl cellulose, hydroxylpropyl cellulose (Klucel® HF), hydroxylpropylmethyl cellulose, hydroxypropyl methylcellulose acetate succinate etc.), synthetic polymers and its derivatives such as without any limitation to carboxyvinyl polymers or carbomers (Carbopol® 940, Carbopol® 934, Carbopol® 971p NF), polyethylene and its copolymers, clays such as but not limited to silicates and bentonite, silicon dioxide, fumed silica (Aerosil®), polyvinyl alcohol, acrylic polymers (Eudragit®), acrylic acid esters, polyacrylate copolymers, polyacrylamide, polyvinyl pyrrolidone homopolymer and polyvinyl pyrrolidone copolymers such as but not limited to (PVP, Kollidon® 30, poloxamer), isobutylene, ethyl vinyl acetate copolymers, natural rubber, synthetic rubber, hot melt adhesives, styrene-butadiene copolymers, bentonite, all water and/or organic solvent swellable polymers, etc. or combinations thereof.

Regardless of the particular thickening agent utilized, the amount of the thickening agent contained in a polymer blend used to form the solubilized drug in adhesive layer, if present, can range from about 0.1 wt. % to about 50 wt. %, such as from about 0.5 wt. % to about 25 wt. %, such as from about 0.75 wt. % to about 15 wt. % based on the dry weight of the solubilized drug in adhesive layer.

e. Skin Permeation Enhancer

The drug in adhesive layer of the transdermal drug delivery system of the present invention can also include one or more suitable surfactants (e.g., non-ionic surfactants), plasticizers, humectants, or a combination thereof that can serve as a skin permeation enhancer to improve the permeation of the immunomodulatory agent through the skin during use of the transdermal drug delivery system. In one particular embodiment, the plasticizer can include various fatty alcohol, fatty acid, and or fatty ester derivatives, such as but not limited to oleic acid, oleyl alcohol, ethyl oleate, oleyl oleate, propylene glycol monolaurate, ethyl ethanoate, isopropyl myristate, myristal alcohol, glyceryl monooleate, lauryl lactate, methyl laurate, phthalate or its derivatives, polyethylene glycol ethers of oleyl alcohol, polyethylene glycol ethers of oleyl alcohol, dodecanol, linoleic acid, lauric acid, lauryl alcohol, isopropyl palmitate, triethyl citrate, triacetin, or humectants, such as glycerin, glycols, diethylene glycol monoethyl ether, or a combination thereof. In one particular embodiment, the skin permeation enhancer can be a non-ionic surfactant that can include fatty derivatives of polyoxyethylene. One example is oleth-3, which is a polyethylene glycol ether of oleyl alcohol having three ethylene oxide units, although other oleths (e.g., −2, −4, −5, −6, −7, −8, −9, −10, −11, −12, −15, −16, −20, −23, −25, −30, −40, −44, and −50), are also contemplated either alone or in combinations thereof. Another non-ionic surfactant that is contemplated is a poloxamer (e.g., P181, P188, P338, P407, or a combination thereof, commercially available as Kolliphor®, Pluronic®, or Lutrol®). Still other non-ionic surfactants that can be utilized include laureths, ceteths, ceteareths, and steareths, either alone or combination with each other or with one or more of the oleths and/or poloxamers referenced above.

In another embodiment permeation enhancers include but not limited to fatty acids such as but not limited to capric acid, caprylic acid, lauric acid, myristic acid, linoleic acid, stearic acid, palmitic acid etc., surfactant type enhancers such as but not limited to Brij®, Tween®, Span®, polysorbate, sorbitan fatty acid esters, or sodium lauryl sulfate, poloxamers, or acids such as salicylic acid.

Regardless of the particular surfactant, plasticizer, humectant, or combinations thereof utilized, the amount of the skin permeation enhancer contained in a polymer blend used to form the solubilized drug in adhesive layer can range from about 1 wt. % to about 80 wt. %, such as from about 5 wt. % to about 60 wt. %, such as from about 10 wt. % to about 40 wt. % based on the dry weight of the solubilized drug in adhesive layer of the transdermal drug delivery system. In one particular embodiment, the skin permeation enhancer can include a combination of oleic acid and isopropyl palmitate, where the ratio of the oleic acid to the isopropyl palmitate can range from 1:1 to about 3:1, such as from about 1.25:1 to 2.5:1, such as from about 1.5:1 to about 2:1.

f. Skin or Adhesive Modifiers

The drug in adhesive layer of the transdermal drug delivery system of the present invention can also include one or more skin or adhesive modifiers, fillers, protectants, antioxidants, ingredients which can reduce or prevent hydrolysis, oxygen scavengers, moisture scavengers, other materials. Suitable skin or adhesive modifiers can include mineral oil, silicone fluid, fatty ester derivatives, phthalate derivatives, butylated hydroxytoluene (BHT), butylated hydroxyanisole (BHA), gallic acid, ascorbic acid, ascorbyl palmitate, lactic acid, methyl salicylate, salicylic acid, derivatives thereof, or a combination thereof.

Regardless of the one or more skin or adhesive modifiers, fillers, protectants, antioxidants, other materials contained in a polymer blend used to form the solubilized drug in adhesive layer, such components can be present in the solubilized drug in adhesive layer in a total amount ranging from about 0.5 wt. % to about 30 wt. %, such as from about 1 wt. % to about 25 wt. %, such as from about 1.5 wt. % to about 20 wt. % based on the dry weight of the solubilized drug in adhesive layer of the transdermal drug delivery system.

g. Polar Aprotic Solvent

The polymer blend from which the solubilized drug in adhesive layer of the transdermal drug delivery system of the present invention can further include one or more polar aprotic solvents, which can assist in the solubility of the immunomodulatory agent in the drug in adhesive polymer blend and in the delivery of the immunomodulatory agent through the skin. A polar aprotic solvent is a solvent that lacks an acidic proton and is polar. Such solvents lack hydroxyl and amine groups. These solvents do not serve as proton donors in hydrogen bonding, although they can be proton acceptors. Specific examples contemplated by the present invention can include n-methyl-2-pyrrolidone (NMP), dimethyl sulfoxide (DMSO), dimethyl isosorbide, ethyl acetate, or a combination thereof, although it is to be understood that other polar aprotic solvents are also contemplated by the present invention, including, but not limited to, acetone, acetonitrile, dichloromethane, dimethylformamide, DMPU, and tetrahydrofuran.

Regardless of the particular polar aprotic solvent or combination of polar aprotic solvents utilized, the total amount of polar aprotic solvent contained in a polymer blend used to form the solubilized drug in adhesive layer can be detectable in the transdermal drug delivery system in an amount less than ICH Q3C Impurities: Guideline for Residual Solvents. For NMP, this equates to levels of less than about 530 parts per million, or less than about 0.053 wt. %, such as less than about 390 parts per millions, or less than about 0.039 wt. %, based on the dry weight of the solubilized drug in adhesive layer where the NMP is to be considered a process solvent. However, it is to be understood that such solvents are introduced in larger wt. % levels during the formation of the solubilized drug in adhesive layer and prior to any evaporation or drying. Further, when utilized as an excipient, the one or more polar aprotic solvents can be present in an amount greater than the ICH Q3C Impurities: Guideline for Residual Solvents. For NMP, this equates to levels greater than about 390 parts per million, or greater than about 0.039 wt. %, such as greater than about 530 parts per million, or greater than about 0.053 wt. % based on the dry weight of the solubilized drug in adhesive layer.

Regardless of the particular polar aprotic solvent or combination of polar aprotic solvents utilized, the total amount of polar aprotic solvent contained in a polymer blend used to form the solubilized drug in adhesive layer can be detectable in the transdermal drug delivery system in an amount less than about 530 parts per million, or less than about 0.053 wt. %, such as less than about 390 parts per millions, or less than about 0.039 wt. %, based on the dry weight of the solubilized drug in adhesive layer. However, it is to be understood that such solvents are introduced in larger wt. % levels during the formation of the solubilized drug in adhesive layer and prior to any evaporation or drying. Further, when utilized as an excipient, the one or more polar aprotic solvents can be present in an amount greater than about 390 parts per million, or greater than about 0.039 wt. %, such as greater than about 530 parts per million, or greater than about 0.053 wt. % based on the dry weight of the solubilized drug in adhesive layer.

In another embodiment, regardless of the particular polar aprotic solvent or combination of polar aprotic solvents utilized, the total amount of polar aprotic solvent contained in a polymer blend used to form the solubilized drug in adhesive layer can be detectable in the transdermal drug delivery system in an amount less than about 20,000 parts per million, or less than about 2.0 wt. %, such as less than about 10,000 parts per millions, or less than about 1.0 wt. %, based on the dry weight of the solubilized drug in adhesive layer. However, it is to be understood that such solvents are introduced in larger wt. % levels during the formation of the solubilized drug in adhesive layer and prior to any evaporation or drying. Further, when utilized as an excipient, the one or more polar aprotic solvents can be present in an amount greater than about 530 parts per million, or greater than about 0.053 wt. %, such as greater than about 10,000 parts per million, or greater than about 1.0 wt. %, such as greater than about 20,000 parts per million, or greater than about 2 wt. %, based on the dry weight of the solubilized drug in adhesive layer.

### II. Backing Layer

Referring again to FIG. 1, in addition to the drug in adhesive layer 110, the transdermal drug delivery system 100 of the present invention can include a backing layer 120 that forms the exterior surface 140 of the transdermal drug delivery system 100. The backing layer 120 can be occlusive and can protect the polymer layer (and any other layers present) from the environment and prevents loss of the drug and/or release of other components to the environment during use. Materials suitable for use as backing layers are well-known known in the art and can comprise films of polyester, polyethylene, vinyl acetate resins, ethylene/vinyl acetate copolymers, polyvinyl chloride, polyurethane, and the like, metal foils, non-woven fabric, cloth and commercially available laminates. A typical backing material has a thickness in the range of 2 to 1000 micrometers. For example, 3M's Scotchpak® such as but not limited to 1012 or 9732 (a polyester film with an ethylene vinyl acetate copolymer heat seal layer), 9723 (a laminate of polyethylene and polyester), 9733, 9735, 9738, or 9754, or CoTran® 9720 (a polyethylene film) are useful in the transdermal drug delivery systems described herein, as are Dow® backing layer films, such as Dow® BLF 2050 (a multi-layer backing comprising ethylene vinyl acetate layers and an internal SARAN® layer.

### III. Release Liner

Referring still to FIG. 1, in addition to the drug in adhesive layer 110 and the backing layer 120, the transdermal drug delivery system 100 of the present invention can also include a release liner 130 disposed on the skin-contacting surface 150 of the transdermal drug delivery system that protects the solubilized drug in adhesive layer 110 of the transdermal drug delivery system 100 until it is ready to be applied to a patient's skin. Once the transdermal drug delivery system 100 is to be applied to a patient's skin at its skin-contacting surface 150, the release liner 130 can be removed and discarded. Materials suitable for use as release liners are well-known known in the art and include the commercially available products of Dow Corning Corporation designated Bio-Release® liner and Syl-off® 7610, Loparex's PET release liner (silicone-coated), Saint Gobain 9011 liner (fluoro-silicone-coated), and 3M's 1020, 1022, 9741, 9744, 9748, 9749 and 9755 Scotchpak® liners, which are fluoropolymer-coated polyester films, or Saint Gobain's liner such as but not limited to 9011.

### IV. Method of Making the Transdermal Drug Delivery System

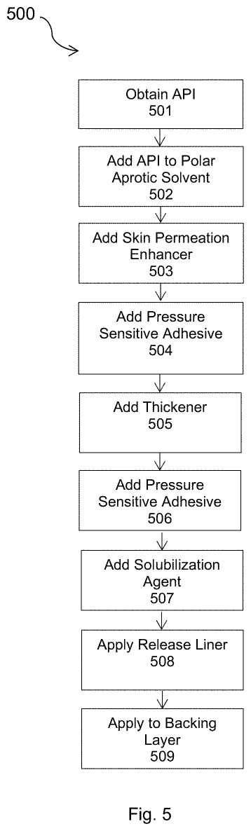

Generally, the drug in adhesive layer of the present invention is made by combining the components in a specific order, resulting in the ability to form a transdermal drug delivery system with a solubilized drug in adhesive layer that exhibits enhanced solubility of an immunomodulatory agent and improved permeation of the immunomodulatory agent through the skin. Referring to FIG. 5, one method 500 of making a polymer blend used to form the solubilized drug in adhesive layer of the present invention is shown. First, in step 501, the API (e.g., an immunomodulatory agent) is obtained. Next, in step 502, the API is added to a polar aprotic solvent to form a solution and kept aside. Then, in step 503 plasticizer or skin permeation enhancer is obtained, followed by a volatile solvent, if needed. Thereafter, in step 505, a thickener can be added. Next, in step 506, the pressure sensitive adhesive can be added. Then, in step 507, a solubilization agent, which can also be referred to as a crystallization inhibitor, can be added to the solution. Next, in step 508 API solution obtained from step 502 is added. In addition, in step 508, after mixing the aforementioned components, the resulting drug in adhesive layer can be applied to and allowed to dry on a release liner on one surface, allowing any volatile solvents present to evaporate, after which a backing layer can be applied to the opposing surface in step 509. Furthermore, the present method 500 contemplates that one or more components of the drug in adhesive layer can be added in any order different from that described above so long as a homogenous, solubilized drug in adhesive layer is formed prior to application of the backing layer and release liner.

Transdermal Drug Delivery System with Solid Dispersion of a Drug in Adhesive Layer

In another embodiment, the present invention is directed to another configuration for a transdermal drug delivery system for the delivery of an immunomodulatory agent through the skin. In one particular embodiment, the immunomodulatory agent can be lenalidomide, although it is to be understood that in alternative embodiments, any other immunomodulatory agent can be utilized in the transdermal drug delivery system. The transdermal drug delivery system includes a solid dispersion of a drug in adhesive layer that includes the immunomodulatory agent (e.g., lenalidomide), a pressure sensitive adhesive, and a binder such as crosslinked polyvinylpyrrolidone. The transdermal drug delivery system may also include a plasticizer or humectant that serves as a skin permeation enhancer, a dispersing agent, a skin and/or adhesive modifier such as a filler, a protectant, an antioxidant, or a combination thereof. Further, the drug in adhesive layer can utilize one or more polar aprotic solvents to ensure that the immunomodulatory agent is solubilized and homogeneously distributed within the drug in adhesive layer which molecularly distributes the drug within a solution and allows adsorption onto substrate particles, such Kollidon® CL-M. When utilized as a process solvent, the a polar aprotic solvent, such as n-methyl-2-pyrrolidone, can be detectable in transdermal drug delivery system in an amount less than about 530 parts per million, or less than about 0.053 wt. %, while when utilized as an excipient, the one or more polar aprotic solvents can be present in an amount greater than about 530 parts per million, or greater than about 0.053 wt. %. Other polar aprotic solvents should be their established residual solvent content based on ICH Q3C Guidelines for Residual Solvents in Drug Products. Without intending to be limited by any particular theory, the present inventors have found that the specific components of the solid dispersion drug in adhesive layer and the method by which the immunomodulatory agent is homogeneously dispersed in the drug in adhesive layer improves its availability and enhances its permeation through the skin.

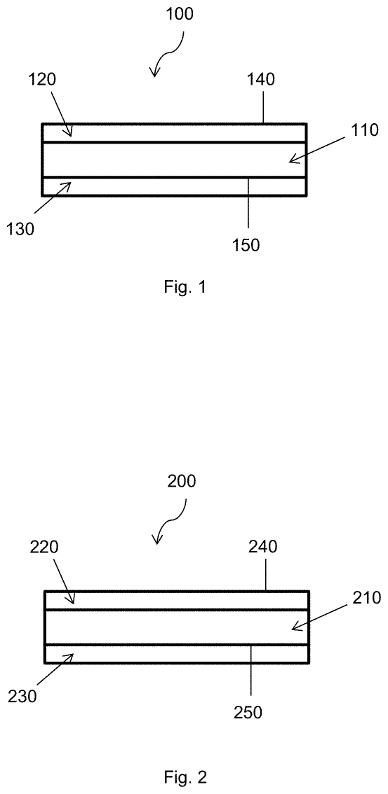

Referring to FIG. 2 and according to one particular embodiment, the transdermal drug delivery system 200 includes a solid dispersion of a drug in adhesive layer 210 disposed between a backing layer 220 and a release liner 230. The backing layer 220 has an exterior surface 240 that is exposed to the ambient environment when the transdermal drug delivery system 200 is in use. Meanwhile, the release liner 230 is positioned on a skin-contacting surface 250 of the solid dispersion drug in adhesive matrix layer 210, where the release liner 230 is removable so that the solid dispersion drug in adhesive layer 210 can be positioned directly on the skin during use of the transdermal drug delivery system 200. As a result of the specific combination of components used to form the solid dispersion of the drug in adhesive layer, such as the particular polar aprotic solvents and the crosslinked polyvinylpyrrolidone, as well as the specific weight percentages and ratios of such components utilized, the present inventors have found that the transdermal drug delivery system 200 can include a stable solid dispersion of the drug in adhesive matrix layer that forms a skin-contacting surface, which facilitates the delivery of the immunomodulatory agent (i.e., the active pharmaceutical ingredient or API) in a controlled manner. For instance, the skin permeation of the immunomodulatory agent can be based on the ratio of the immunomodulatory agent to the binder (e.g., cross-linked polyvinylpyrrolidone). As shown in FIG. 2, the solid dispersion drug in adhesive layer 210 can be in the form of a single layer so that the active pharmaceutical ingredient is homogeneously dispersed throughout adhesive component of the device 200. However, it should also be understood that additional solid dispersion drug in adhesive layers may also be included in the transdermal drug delivery system 200.

The various components of the transdermal drug delivery system 100 are discussed in detail below.

### I. Solid Dispersion of the Drug in Adhesive Layer

a. Active Pharmaceutical Ingredient

The polymer blend use to form the solid dispersion of the drug in adhesive layer of the transdermal drug delivery system of the present invention can include any suitable drug or active pharmaceutical ingredient (API) that functions as an immunomodulatory agent. For instance, the immunomodulatory agent can include all pharmaceutically acceptable forms of an immunomodulatory imide compound, such as thalidomide, including analogs of thalidomide including lenalidomide, pomalidomide, and iberdomide including, for example, free base, salts, polymorphs, solvates, solutions, isomers, amorphous, crystalline, co crystalline, solid solution, prodrugs, analogs, derivatives, and metabolites and combinations thereof. The compound may be in the form of a pharmaceutically acceptable salt, such as an acid addition salt or a base salt, or a solvate thereof, including a hydrate thereof. Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methane sulphonate, ethanesulphonate, benzenesulphonate, p-toluenesulphonate and pamoate salts.

Regardless of the particular immunomodulatory agent utilized as the API, the amount of the API contained in a polymer blend used to form the solid dispersion drug in adhesive layer can range from about 0.1 wt. % to about 25 wt. %, such as from about 0.5 wt. % to about 20 wt. %, such as from about 0.75 wt. % to about 15 wt. % based on the dry weight of the solid dispersion of the drug in adhesive layer.

The API may be amorphous prior to incorporation into a solid dispersion or may be dissolved into an appropriate solvent for the API, such as a polar aprotic solvent (e.g., n-methyl-2-pyrrolidone for lenalidomide) at a higher concentration than that intended for the final concentration in polymer blend. For instance, to achieve a weight percentage of about 0.5 wt. % to about 20 wt. % of the API in a final product such as in a solid dispersion of a drug in adhesive layer, a weight percentage of about 10 wt. % to about 50 wt. % of LLD in NMP solution can be used based on the wet weight of the API in the solvent, wherein the saturation is greater than about 5 wt. % of LLD in NMP.

b. Pressure Sensitive Adhesive

The solid dispersion drug in adhesive layer of the transdermal drug delivery system of the present invention also includes one or more suitable pressure sensitive adhesive (PSA). Adhesive polymers may be made from various materials which include plastics, polymers, pressure sensitive adhesives, self-adhering systems, or may require additional excipients to obtain pressure sensitive properties. Basic adhesive systems are selected from polyacrylics, silicones, polyisobutylenes, rubbers, and combinations thereof either by physical blending or copolymerization is disclosed. These materials may be obtained from solvent-borne, water-borne, physical mixtures, extruded, co-extruded, hot melt, or otherwise formed as polymerized or unpolymerized materials.

In one embodiment, the PSA can be an acrylic polymer. Useful acrylic polymers include various homopolymers, copolymers, terpolymers and the like of acrylic acids and derivatives thereof as a cross-linked, cross-linkable, uncross-linked, uncross-linkable, grafted, block, cured and non-curing pressure sensitive adhesives (PSAs). These acrylic polymers include copolymers of alkyl acrylates or methacrylates. Polyacrylates include acrylic acid, methacrylic acid, and derivatives thereof without limitation, methyl acylate, methyl methacrylate, ethyl acrylate, ethyl methacrylate, n-butyl acrylate, n-butyl methacrylate, hexyl acrylate, 2-ethylbutyl acrylate, isooctyl acrylate, 2-ethylhexyl acrylate, 2-ethylhexyl methacrylate, decyl acrylate, decylmethacrylate, dodecyl acrylate, dodecyl methacrylate, tridecyl acrylate, tridecyl methacrylate, vinyl acetate, 2-hydroxyethyl acrylate, glycidyl methacrylate, or octylacrylamide. The acrylic polymer may be functional species with levels of hydroxyl or carboxyl moieties or combinations thereof, non-functional species without functional moieties, non-reactive species with moieties which are less reactive than hydroxyl or carboxyl moieties, such as methyl or ethyl or propyl or butyl capped acrylamides. Exemplary acrylic PSAs include, without limitation, one or more of: Duro-Tak® 87-900A, Duro-Tak 87-9301, Duro-Tak® 87-4098, Duro-Tak® 387-2510/87-2510, Duro-Tak® 387-2287/87-2287, Duro-Tak® 87-4287, Duro-Tak® 387-2516/87-2516, Duro-Tak® 87-2074, Duro-Tak® 87-235A, Duro-Tak 387-2353/87-2353, Gelva® GMS 9073, Duro-Tak® 87-2852, Duro-Tak® 387-2051/87-2051, Duro-Tak® 387-2052/87-2052, Duro-Tak® 387-2054/87-2054, Duro-Tak® 87-2194, or Duro-Tak® 87-2196. It should also be understood that the disclosure herewith incorporates known and unknown naming conventions comprising the monomers disclosed.

In one particular embodiment, the present inventors have found that the use of a PSA that includes an acrylate copolymer without having —COOH or —OH functional groups contributes to the improved permeation of the immunomodulatory agent contained in the drug in adhesive layer. Further, it has also been found that an acrylate copolymer having a solids content ranging from about 30% to about 55%, such as from about 35% to about 50%, such as from about 36% to about 45% also contributes to the improved solubility and permeation of the immunomodulatory agent. Additionally, an acrylate copolymer having a viscosity of less than about 6500 centipoise, such as from about 2000 centipoise to about 5000 centipoise, such as from about 2500 centipoise to about 4500 centipoise may also contribute to the improved solubility and permeation of the immunomodulatory agent, where the viscosity impacts the loading capacity of the components in polymer blend used to form the drug in adhesive matrix layer. Further, an acrylate copolymer that includes vinyl acetate may also be beneficial.

Particular examples include Duro-Tak® 387-2516/87-2516 (vinyl acetate; —OH functional groups; 41.5% solids; viscosity of 4350 centipoise), Duro-Tak® 387-2052/87-2052 (vinyl acetate; —COOH functional groups, 47.5% solids; viscosity of 2750 centipoise), or Duro-Tak® 87-4098 (vinyl acetate; 38.5% solids content; viscosity of 6500 centipoise).

In still another embodiment, the PSA can include silicone. Suitable silicone adhesives include pressure sensitive adhesives made from silicone polymer and resin. The polymer to resin ratio can be varied to achieve different levels of tack. Specific examples of useful silicone adhesive which are commercially available include the standard BIO-PSA® series (7-4400, 7-4500, and 7-4600 series) and the amine compatible (end capped) BIO-PSA® series (7-4100, 7-4200, and 7-4300 series) manufactured by Dow Corning. Preferred adhesives include BIO-PSA® 7-4101, 7-4102, 7-4201, 7-4202, 7-4301, 7-4302, 7-4401, 7-4402, 7-4501, 7-4502, 7-4601, and 7-4602.

In still another embodiment, the PSA can include polyisobutylene. Suitable polyisobutylene adhesives are those which are pressure sensitive and have suitable tack. The polyisobutylene can comprise a mixture of high and medium molecular weight polyisobutylenes, polybutenes, and mineral oils. Specifically, high molecular weight polyisobutylenes are those with a molecular weight of at least about 425,000. Medium molecular weight polyisobutylenes are those with a molecular weight of at least 40,000 but less than about 425,000. Low molecular weight polyisobutylenes are those with a molecular weight of at least 100 but less than about 40,000. Specific examples of useful polyisobutylene adhesives which are commercially available include Oppanol® High Molecular Weight N grades 50, 50SF, 80, 100 and 150, and Oppanol® Medium Molecular Weight B grades 10N, 10SFN, 11SFN, 12SFN, 12N, 13SFN, 14SFN, 15SFN, and 15N manufactured by BASF. Specific examples of polybutenes are commercially available from Soltex as polybutenes of various molecular weights and by Ineos as Indopol and Panalane with various molecular weights. A specific example of a useful polyisobutylene adhesive which is commercially available includes Duro-Tak® 87-6908.

Other pressure sensitive adhesives obtained from rubber block copolymers, such as Styrene-Isoprene-Styrene (SIS) or Styrene-Butadiene-Styrene (SBS, based adhesives are also contemplated by the present invention.

Regardless of the particular PSA utilized, the pressure sensitive adhesive can be present in an amount ranging from about 1 wt. % to about 99 wt. %, such as from about 20 wt. % to about 99 wt. %, such as from about 40 wt. % to about 98 wt. % based on the dry weight of the solid dispersion drug in adhesive layer.

c. Binder

The solid dispersion drug in adhesive layer of the transdermal drug delivery system of the present invention can also include a binder that is a micronized crosslinked polyvinylpyrrolidone (PVP), such as a crosslinked homopolymer of N-vinyl-2-pyrrolidone, which can allow molecular adsorption of the AP onto a solid porous substrate in the adhesive layer. In one particular embodiment, the crosslinked PVP is in the form of a water-insoluble powder. Such cross-linked PVPs are commercially available under the name Kollidon®, available from BASF. A specific example of a cross-linked PVP that is contemplated for use in the present invention is Kollidon® CL-M. Other cross-linked PVPs that can be used include Kollidon® CL-SF and CL-F. The cross-linked PVP can be micronized and can have an average particle size ranging from about 1 micrometer to about 40 micrometers, such as from about 2 micrometers to about 30 micrometers, such as from about 3 micrometers to about 10 micrometers. In addition, in one particular embodiment, greater than 90% of the particles utilized can have a particle size less than about 15 micrometers. The particle size of the cross-linked PVP contemplated for use in the drug-in-adhesive matrix layer of the present invention is thus smaller than typical cross-linked PVPs, which can have particle sizes up to 150 micrometers. Without intending to be limited by any particular theory, the present inventors have found that utilizing a cross-linked PVP where greater than 90% of the particles have a particle size less than about 15 micrometers can result in the formation of a stable polymer blend that is used to form the solid dispersion drug in adhesive layer, where the API is maintained in a uniform suspension with minimal sedimentation. This, in turn, enables the formation of a homogeneous dispersion of the API in the drug in adhesive layer so that the transdermal drug delivery system can deliver the API in a controlled manner through the skin.

Moreover, the crosslinked PVP particles contemplated for use in the present invention can have a bulk density ranging from about 0.10 g/mL to about 0.40 g/mL, such as from about 0.125 g/mL to about 0.35 g/mL, such as from about 0.15 g/mL to about 0.25 g/mL. Further, the crosslinked PVP particles can have a surface area ranging from about 0.5 m²/g to about 20 m²/g, such as from about 1 m²/g to about 15 m²/g, such as from about 1.5 m²/g to about 10 m²/g. The increased surface area of the cross-linked PVP particles contemplated for use in the present invention can facilitate the dispersion of the API in a uniform, homogeneous manner throughout the solid dispersion drug in adhesive layer, which enables the API to be delivered at a constant rate.

The amount of the crosslinked polyvinylpyrrolidone contained in the solid dispersion drug in adhesive layer can range from about 0.1 wt. % to about 40 wt. %, such as from about 1.5 wt. % to about 20 wt. %, such as from about 2 wt. % to about 15 wt. % based on the dry weight of the solid dispersion drug in adhesive layer. Further, the present inventors have found that the ratio of the immunomodulatory agent to the crosslinked polyvinylpyrrolidone impacts the formation of the solid dispersion, where a ratio of the immunomodulatory agent to the crosslinked polyvinylpyrrolidone that ranges from about 1:10 to about 4:1, such as from about 1:5 to about 2:1, such as from about 1:3 to about 1:1 facilitates formation of the solid dispersion. In another embodiment, the ratio of the immunomodulatory agent to the crosslinked polyvinylpyrrolidone that ranges from about 1:1 to about 1:6, such as from about 1:1.5 to about 1:4, such as from about 1:2 to about 1:3 results in increased flux of the immunomodulatory agent through the skin.

d. Dispersing Agent

The solid dispersion drug in adhesive layer of the transdermal drug delivery system of the present invention can also include one or more dispersing agents. The one or more thickening agents can include those that do not exhibit solubility for the immunomodulatory agent. For instance, the dispersing agent can include mineral oil, silicone fluid, fatty acid esters, or a combination thereof.

Regardless of the particular dispersing agent utilized, the amount of the dispersing agent contained in the solid dispersion drug in adhesive layer, if present, can range from about 0.1 wt. % to about 25 wt. %, such as from about 0.5 wt. % to about 20 wt. %, such as from about 0.75 wt. % to about 15 wt. % based on the dry weight of the solid dispersion drug in adhesive layer.

e. Skin Permeation Enhancers

The solid dispersion drug in adhesive layer of the transdermal drug delivery system of the present invention can also include one or more suitable surfactants (e.g., non-ionic surfactants), plasticizers, humectants or a combination thereof that can serve as a skin permeation enhancer to improve the permeation of the immunomodulatory agent through the skin during use of the transdermal drug delivery system.

In one particular embodiment, the skin permeation enhancer can be a surfactant, such as, but not limited to, a non-ionic surfactant. In one particular embodiment, the non-ionic surfactant can include various fatty alcohol, fatty acid, and/or fatty ester derivatives, such as oleic acid, oleyl alcohol, ethyl oleate, oleyl oleate, polyethylene glycol ethers of oleyl alcohol, linoleic acid, lauric acid, lauryl alcohol, lauryl lactate, myristic alcohol, isopropyl palmitate, etc. or humectants (glycerin, triethyl citrate, triacetin, glycols, diethylene glycol monoethyl ether, PEG, etc.) or a combination thereof. In one particular embodiment, the skin permeation enhancer can be a non-ionic surfactant that can include fatty derivatives of polyoxyethylene. One example is oleth-3, which is a polyethylene glycol ether of oleyl alcohol having three ethylene oxide units, although other oleths (e.g., −2, −4, −5, −6, −7, −8, −9, −10, −11, −12, −15, −16, −20, −23, −25, −30, −40, −44, and −50) are also contemplated either alone or in combinations thereof. Without intending to be limited by any particular theory, is believed that the oleth contributes to an increase in flux and the ability of the system to overcome a barrier of drop in flux 24-hours post application to the skin. Another non-ionic surfactant that is contemplated is a poloxamer (e.g., P181, P188, P338, P407, or a combination thereof, commercially available as Kolliphor®, Pluronic®, or Lutrol®). Still other non-ionic surfactants that can be utilized include laureths, ceteths, ceteareths, and steareths, either alone or combination with each other or with one or more of the oleths and/or poloxamers referenced above.

Regardless of the particular non-ionic surfactant, plasticizer, humectant, or combinations thereof utilized, the amount of the skin permeation enhancer contained in a polymer blend used to form the drug in adhesive layer can range from about 1 wt. % to about 60 wt. %, such as from about 5 wt. % to about 40 wt. %, such as from about 10 wt. % to about 30 wt. % based on the dry weight of the drug in adhesive layer of the transdermal drug delivery system. In one particular embodiment, the skin permeation enhancer can include from about 0.5 wt. % to about 30 wt. %, such as from about 2.5 wt. % to about 25 wt. %, such as from about 5 wt. % to about 20 wt. % of a first non-ionic surfactant (e.g., one or more polyethylene glycol ethers of oleyl alcohol such as oleth-3 in combination with oleth-2, -5, -10, and/or -20, or oleth-5 in combination with oleth-2, -3, -10, and/or -20) and from about 0.5 wt. % to about 30 wt. %, such as from about 2.5 wt. % to about 25 wt. %, such as from about 5 wt. % to about 20 wt. % of a second non-ionic surfactant (e.g., a poloxamer, such as P407), where it has been found that the use of such a combination of skin permeation enhancers can result in a transdermal drug delivery system that exhibits significantly sustained delivery of the API.

f. Skin or Adhesive Modifiers

The solid dispersion drug in adhesive layer of the transdermal drug delivery system of the present invention can also include one or more skin or adhesive modifiers, fillers, protectants, antioxidants, other materials. Suitable skin or adhesive modifiers can include butylated hydroxytoluene (BHT), butylated hydroxyanisole (BHA), gallic acid, ascorbic acid, ascorbyl palmitate, lactic acid, methyl salicylate, salicylic acid or a combination thereof.

Regardless of the one or more skin or adhesive modifiers, fillers, protectants, antioxidants, other materials contained in a polymer blend used to form the solid dispersion drug in adhesive layer, such components can be present in the solid dispersion drug in adhesive layer in a total amount ranging from about 0.25 wt. % to about 10 wt. %, such as from about 0.5 wt. % to about 7.5 wt. %, such as from about 1 wt. % to about 5 wt. % based on the dry weight of the solid dispersion drug in adhesive layer of the transdermal drug delivery system.

g. Polar Aprotic Solvent

The polymer blend from which the solid dispersion drug in adhesive layer of the transdermal drug delivery system of the present invention can further include one or more polar aprotic solvents, which can be used to adjust the solids content of the pressure sensitive adhesive and assist in the dispersion of the immunomodulatory agent in the drug in adhesive polymer blend and in the delivery of the immunomodulatory agent through the skin. The present inventors have found that without solubilizing the immunomodulatory agent (LLD) prior to addition, the precipitation onto the crosslinked polyvinylpyrrolidone does not occur, and availability of the drug is not allowed for permeation.

A polar aprotic solvent is a solvent that lacks an acidic proton and is polar. Such solvents lack hydroxyl and amine groups. These solvents do not serve as proton donors in hydrogen bonding, although they can be proton acceptors. Specific examples contemplated by the present invention can include n-methyl-2-pyrrolidone (NMP), dimethyl sulfoxide (DMSO), dimethyl isosorbide, ethyl acetate, or a combination thereof, although it is to be understood that other polar aprotic solvents are also contemplated by the present invention, including, but not limited to, acetone, acetonitrile, dichloromethane, dimethylformamide, DMPU, and tetrahydrofuran.

Regardless of the particular polar aprotic solvent or combination of polar aprotic solvents utilized, the total amount of polar aprotic solvent contained in a polymer blend used to form the solid dispersion drug in adhesive layer can be detectable in the transdermal drug delivery system in an amount less than ICH Q3C Impurities: Guideline for Residual Solvents. For NMP, this equates to levels of less than about 530 parts per million, or less than about 0.053 wt. %, such as less than about 390 parts per millions, or less than about 0.039 wt. %, based on the dry weight of the solid dispersion drug in adhesive layer where the NMP is to be considered a process solvent. However, it is to be understood that such solvents are introduced in larger wt. % levels during the formation of the solid dispersion drug in adhesive layer and prior to any evaporation or drying. Further, when utilized as an excipient, the one or more polar aprotic solvents can be present in an amount greater than the ICH Q3C Impurities: Guideline for Residual Solvents. For NMP, this equates to levels greater than about 390 parts per million, or greater than about 0.039 wt. %, such as greater than about 530 parts per million, or greater than about 0.053 wt. % based on the dry weight of the solid dispersion drug in adhesive layer.

In another embodiment, regardless of the particular polar aprotic solvent or combination of polar aprotic solvents utilized, the total amount of polar aprotic solvent contained in a polymer blend used to form the solubilized drug in adhesive layer can be detectable in the transdermal drug delivery system in an amount less than about 20,000 parts per million, or less than about 2.0 wt. %, such as less than about 10,000 parts per millions, or less than about 1.0 wt. %, based on the dry weight of the solid dispersion drug in adhesive layer. However, it is to be understood that such solvents are introduced in larger wt. % levels during the formation of the solid dispersion drug in adhesive layer and prior to any evaporation or drying. Further, when utilized as an excipient, the one or more polar aprotic solvents can be present in an amount greater than about 530 parts per million, or greater than about 0.053 wt. %, such as greater than about 10,000 parts per million, or greater than about 1.0 wt. %, such as greater than about 20,000 parts per million, or greater than about 2 wt. %, based on the dry weight of the solid dispersion drug in adhesive layer.

### II. Backing Layer

Referring again to FIG. 2, in addition to the solid dispersion drug in adhesive layer 210, the transdermal drug delivery system 200 of the present invention can include a backing layer 220 that forms the exterior surface 240 of the transdermal drug delivery system 200. The backing layer 220 can be occlusive in nature and can protect the polymer layer (and any other layers present) from the environment and prevents loss of the drug and/or release of other components to the environment during use. Materials suitable for use as backing layers are well-known known in the art and can comprise films of polyester, polyethylene, vinyl acetate resins, ethylene/vinyl acetate copolymers, polyvinyl chloride, polyurethane, and the like, metal foils, non-woven fabric, cloth and commercially available laminates. A typical backing material has a thickness in the range of 2 to 1000 micrometers. For example, 3M's Scotchpak® 1012 or 9732 (a polyester film with an ethylene vinyl acetate copolymer heat seal layer), 9723 (a laminate of polyethylene and polyester), 9754 (a polyester film backing laminate), or CoTran® 9720 (a polyethylene film) are useful in the transdermal drug delivery systems described herein, as are Dow® backing layer films, such as Dow® BLF 2050 (a multi-layer backing comprising ethylene vinyl acetate layers and an internal SARAN® layer.

### III. Release Liner

Referring still to FIG. 2, in addition to the solid dispersion drug in adhesive layer 210 and the backing layer 220, the transdermal drug delivery system 200 of the present invention also includes a release liner 230 disposed on the skin-contacting surface 250 of the transdermal drug delivery system that protects the solid dispersion drug in adhesive matrix layer 210 of the transdermal drug delivery system 200 until it is ready to be applied to a patient's skin. Once the transdermal drug delivery system 200 is to be applied to a patient's skin at its skin-contacting surface 250, the release liner 230 can be removed and discarded. Materials suitable for use as release liners are well-known known in the art and include the commercially available products of Dow Corning Corporation designated Bio-Release® liner and Syl-off® 7610, Loparex's PET release liner (silicone-coated), Saint Gobaine's 9011 liner, and 3M's 1020, 1022, 9741, 9744, 9748, 9749, and 9755 Scotchpak® liners, which are fluoropolymer-coated polyester films.

### IV. Method of Making the Transdermal Drug Delivery System

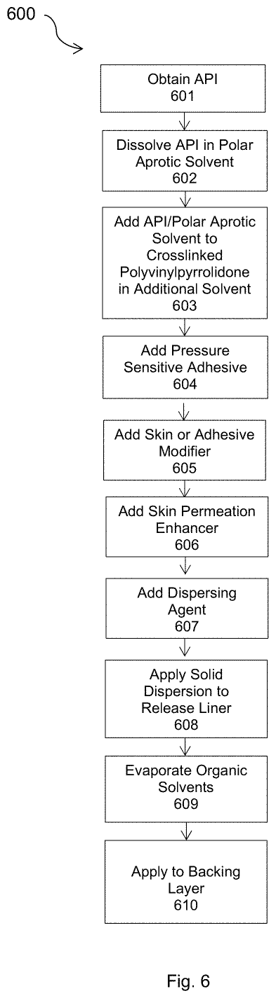

Generally, the solid dispersion of a drug in adhesive layer of the present invention is made by combining the components in a specific order, resulting in the ability to form a transdermal drug delivery system with a solid dispersion of the drug in adhesive layer that exhibits improved permeation of the immunomodulatory agent through the skin. Referring to FIG. 6, one method 600 of making a polymer blend used to form the solid dispersion of the drug in adhesive layer of the present invention is shown. First, in step 601, the API (e.g., an immunomodulatory agent) is obtained. Next, in step 602, the API is added to a polar aprotic solvent (e.g., n-methyl-2-pyrrolidone) to form a solution. Then, in step 603, the API/polar aprotic solvent can be added to a solution of crosslinked polyvinylpyrrolidone that is in an additional solvent (e.g., ethyl acetate). Next, in step 604, the pressure sensitive adhesive can be added. Thereafter, in step 605, a skin or adhesive modifier can be added. Next, in step 606, a skin permeation enhancer can be added. In addition, in step 607 a dispersing agent can be added, although it is to be understood that steps 602 through 607 can be carried out in any suitable order. Then, in step 608, after mixing the aforementioned components, the resulting solid dispersion of the drug in adhesive layer can be applied to and allowed to dry on a release liner on one surface, after which any organic solvents present can be allowed to evaporate in step 609. Then, the opposing surface of the solid dispersion of the drug in adhesive layer can be applied to (e.g., laminated to) a backing layer in step 610, where it is to be understood that the backing layer should be occlusive in nature. Thereafter, individual transdermal drug delivery systems can be die-cut from a large sheet of the formed transdermal drug delivery system, either with or without an inherent overlay system that ensures adhesion to a patient, where it is understood that inherent overlay systems are not drug-bearing and can be non-woven/non-occlusive or occlusive in nature. Furthermore, the present method 600 contemplates that one or more components of the drug in adhesive layer can be added in any order different from that described above so long as a homogenous, solubilized drug in adhesive layer is formed prior to application of the backing layer and release liner.

Transdermal Drug Delivery System with Separate Adhesive and Drug in Polymer Layers

It is believed that it is necessary to have a continuous delivery of lenalidomide and other immunomodulatory agents in order to have a therapeutic effect with minimum adverse or side effects. In one embodiment, the present invention contemplates a multilayer adhesive and polymer matrix formulation to provide continuous delivery of LLD through the transdermal route for a period of up to about seven days. The average flux can range from about 1.5 micrograms per square centimeters per hour to about 6 micrograms per square centimeters per hour, such as from about 1.75 micrograms per square centimeters per hour to about 5.5 micrograms per square centimeters per hour, such as from about 2 micrograms per square centimeters per hour to about 5 micrograms per square centimeters per hour over a course of up to about 72 hours.

In one embodiment, the present invention is directed to a transdermal drug delivery system for the delivery of an immunomodulatory agent through the skin. In one particular embodiment, the immunomodulatory agent can be lenalidomide, although it is to be understood that in alternative embodiments, any other immunomodulatory agent can be utilized in the transdermal drug delivery system. The transdermal drug delivery system includes a separate adhesive layer and a separate drug containing layer that includes the immunomodulatory agent (e.g., lenalidomide) and a solubilizing agent or a crystallization inhibitor. The drug containing layer may also include a plasticizer or humectant that serves as a skin permeation enhancer, a thickener, a skin and/or adhesive modifier such as a filler, a protectant, an antioxidant, or a combination thereof. Further, the drug containing layer can utilize one or more polar aprotic solvents to ensure that the immunomodulatory agent is solubilized and homogeneously distributed within the drug containing layer. When utilized as a process solvent, the one or more polar aprotic solvents can be detectable in transdermal drug delivery system in an amount less than about 530 parts per million, or less than about 0.053 wt. %, while when utilized as an excipient, the one or more polar aprotic solvents can be present in an amount greater than about 530 parts per million, or greater than about 0.053 wt. %. Without intending to be limited by any particular theory, the present inventors have found that the specific components of the adhesive layer and drug containing layer and the method by which the immunomodulatory agent is solubilized in the drug containing improves its solubility in the blend and enhances its permeation through the skin.

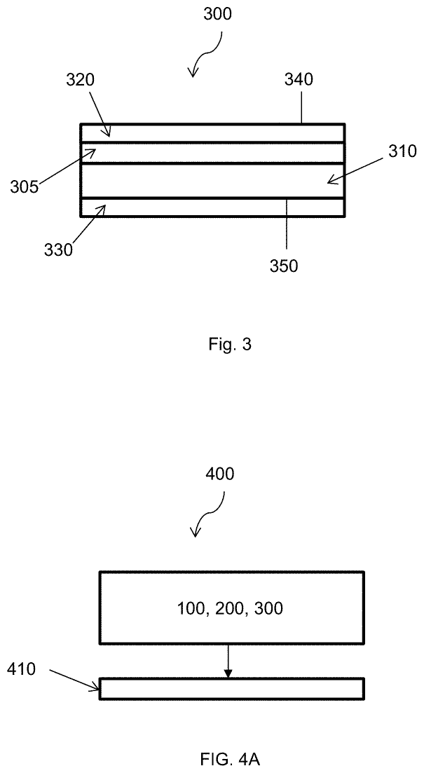

Referring to FIG. 3 and according to one particular embodiment, the transdermal drug delivery system 300 includes a drug containing layer 310 disposed between a backing layer 320 and a release liner 330. Further, a separate adhesive layer 305 is disposed between the drug containing layer 310 and the backing layer 320. The backing layer 320 has an exterior surface 340 that is exposed to the ambient environment when the transdermal drug delivery system 300 is in use. Meanwhile, the release liner 330 is positioned on a skin-contacting surface 350 of the drug containing layer 310, where the release liner 330 is removable so that the drug containing layer 310 can be positioned directly on the skin during use of the transdermal drug delivery system 300.

As a result of the specific combination of components in the drug containing layer, such as the particular polar aprotic solvents and solubilization agent or crystallization inhibitors, as well as the specific weight percentages and ratios of such components utilized, the present inventors have found that the transdermal drug delivery system 300 can include non-drug containing layer and a drug-containing layer that acts as a reservoir forms a skin-contacting surface, which facilitates the delivery of the immunomodulatory agent (i.e., the active pharmaceutical ingredient or API) in a controlled manner for up to about seven days. As shown in FIG. 3, the drug containing layer 310 can be in the form of a single layer so that the active pharmaceutical ingredient is homogeneously dispersed throughout adhesive component of the device 300. However, it should also be understood that additional drug containing layers may also be included in the transdermal drug delivery system 300.

The various components of the transdermal drug delivery system 300 are discussed in detail below.

### I. Non-Drug Containing Layer

a. Pressure Sensitive Adhesive

The non-drug containing layer of the transdermal drug delivery system of the present invention includes one or more suitable pressure sensitive adhesive (PSA). Adhesive polymers may be made from various materials which include plastics, polymers, pressure sensitive adhesives, self-adhering systems, or may require additional excipients to obtain pressure sensitive properties. Basic adhesive systems are selected from polyacrylics, silicones, polyisobutylenes, rubbers, and combinations thereof either by physical blending or copolymerization is disclosed. These materials may be obtained from solvent-borne, water-borne, physical mixtures, extruded, co-extruded, hot melt, or otherwise formed as polymerized or unpolymerized materials.

In one embodiment, the PSA can be an acrylic polymer. Useful acrylic polymers include various homopolymers, copolymers, terpolymers and the like of acrylic acids and derivatives thereof as a cross-linked, cross-linkable, uncross-linked, uncross-linkable, grafted, block, cured and non-curing pressure sensitive adhesives (PSAs). These acrylic polymers include copolymers of alkyl acrylates or methacrylates. Polyacrylates include acrylic acid, methacrylic acid, and derivatives thereof without limitation, methyl acylate, methyl methacrylate, ethyl acrylate, ethyl methacrylate, n-butyl acrylate, n-butyl methacrylate, hexyl acrylate, 2-ethylbutyl acrylate, isooctyl acrylate, 2-ethylhexyl acrylate, 2-ethylhexyl methacrylate, decyl acrylate, decylmethacrylate, dodecyl acrylate, dodecyl methacrylate, tridecyl acrylate, tridecyl methacrylate, vinyl acetate, 2-hydroxyethyl acrylate, glycidyl methacrylate, or octylacrylamide. The acrylic polymer may be functional species with levels of hydroxyl or carboxyl moieties or combinations thereof, non-functional species without functional moieties, non-reactive species with moieties which are less reactive than hydroxyl or carboxyl moieties, such as methyl or ethyl or propyl or butyl capped acrylamides. Exemplary acrylic PSAs include, without limitation, one or more of: Duro-Tak® 87-900A, Duro-Tak 87-9301, Duro-Tak® 87-4098, Duro-Tak® 387-2510/87-2510, Duro-Tak® 387-2287/87-2287, Duro-Tak® 87-4287, Duro-Tak® 387-2516/87-2516, Duro-Tak® 87-2074, Duro-Tak® 87-235A, Duro-Tak 387-2353/87-2353, Gelva® GMS 9073, Duro-Tak® 87-2852, Duro-Tak® 387-2051/87-2051, Duro-Tak® 387-2052/87-2052, Duro-Tak® 387-2054/87-2054, Duro-Tak® 87-2194, or Duro-Tak® 87-2196. It should also be understood that the disclosure herewith incorporates known and unknown naming conventions comprising the monomers disclosed. In one embodiment, the pressure sensitive adhesive can be an acrylate copolymer with no functional groups and having a viscosity greater than about 6000 centipoise, such as from about 6500 centipoise to about 10,000 centipoise, can be utilized based on its low solubility with LLD and its compatibility with the high hydrophilic drug containing polymer blend/layer. In one particular embodiment, the pressure sensitive adhesive can be Duro-Tak® 87-9301, an acrylate polymer with no functional groups and having a viscosity of about 9500 centipoise.

Particular examples include Duro-Tak® 387-2516/87-2516 (vinyl acetate; —OH functional groups; 41.5% solids; viscosity of 4350 centipoise), Duro-Tak® 387-2052/87-2052 (vinyl acetate; —COOH functional groups, 47.5% solids; viscosity of 2750 centipoise), or Duro-Tak® 87-4098 (vinyl acetate; 38.5% solids content; viscosity of 6500 centipoise).

In still another embodiment, the PSA can include silicone. Suitable silicone adhesives include pressure sensitive adhesives made from silicone polymer and resin. The polymer to resin ratio can be varied to achieve different levels of tack. Specific examples of useful silicone adhesive which are commercially available include the standard BIO-PSA® series (7-4400, 7-4500, and 7-4600 series) and the amine compatible (end capped) BIO-PSA® series (7-4100, 7-4200, and 7-4300 series) manufactured by Dow Corning. Preferred adhesives include BIO-PSA® 7-4101, 7-4102, 7-4201, 7-4202, 7-4301, 7-4302, 7-4401, 7-4402, 7-4501, 7-4502, 7-4601, and 7-4602.

In still another embodiment, the PSA can include polyisobutylene. Suitable polyisobutylene adhesives are those which are pressure sensitive and have suitable tack. The polyisobutylene can comprise a mixture of high and medium molecular weight polyisobutylenes, polybutenes, and mineral oils. Specifically, high molecular weight polyisobutylenes are those with a molecular weight of at least about 425,000. Medium molecular weight polyisobutylenes are those with a molecular weight of at least 40,000 but less than about 425,000. Low molecular weight polyisobutylenes are those with a molecular weight of at least 100 but less than about 40,000. Specific examples of useful polyisobutylene adhesives which are commercially available include Oppanol® High Molecular Weight N grades 50, 50SF, 80, 100 and 150, and Oppanol® Medium Molecular Weight B grades 10N, 10SFN, 11SFN, 12SFN, 12N, 13SFN, 14SFN, 15SFN, and 15N manufactured by BASF. Specific examples of polybutenes are commercially available from Soltex as polybutenes of various molecular weights and by Ineos as Indopol and Panalane with various molecular weights. A specific example of a useful polyisobutylene adhesive which is commercially available includes Duro-Tak® 87-6908.

Other pressure sensitive adhesives obtained from rubber block copolymers, such as Styrene-Isoprene-Styrene (SIS) or Styrene-Butadiene-Styrene (SBS, based adhesives are also contemplated by the present invention.

Regardless of the particular PSA utilized, the pressure sensitive adhesive can be present in an amount ranging from about 1 wt. % to about 99 wt. %, such as from about 20 wt. % to about 98.5 wt. %, such as from about 40 wt. % to about 98 wt. % based on the dry weight of the entire transdermal drug delivery system.

### II. Drug Containing Solid Polymer Film Layer

a. Active Pharmaceutical Ingredient

The drug containing layer of the transdermal drug delivery system of the present invention can be in the form of a solid polymer film and can include any suitable drug or active pharmaceutical ingredient (API) that functions as an immunomodulatory agent. For instance, the immunomodulatory agent can include all pharmaceutically acceptable forms of an immunomodulatory imide compound, such as thalidomide, including analogs of thalidomide including lenalidomide, pomalidomide, and iberdomide including, for example, free base, salts, polymorphs, solvates, solutions, isomers, amorphous, crystalline, co crystalline, solid solution, prodrugs, analogs, derivatives, and metabolites and combinations thereof. The compound may be in the form of a pharmaceutically acceptable salt, such as an acid addition salt or a base salt, or a solvate thereof, including a hydrate thereof. Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methane sulphonate, ethanesulphonate, benzenesulphonate, p-toluenesulphonate and pamoate salts.

Regardless of the particular immunomodulatory agent utilized as the API, the amount of the API contained in the drug containing polymer layer can range from about 0.1 wt. % to about 50 wt. %, such as from about 0.5 wt. % to about 35 wt. %, such as from about 0.75 wt. % to about 20 wt. % based on the dry weight of the drug containing polymer layer.

b. Solubilization Agent/Crystallization Inhibitor

The drug containing polymer layer of the transdermal drug delivery system of the present invention can also include one or more solubilization agents or crystallization inhibitors that can include polyvinylpyrrolidone (PVP), such as uncrosslinked PVP. Without intending to be limited by any particular theory, the present inventors have found that the uncrosslinked PVP may function in a polar aprotic nature by structure, the polymer contains a 5-member ring with a tertiary amine and a ketone in a specific arrangement. Thereby, this type of polymer avoids the use of an alcohol (—OH) group excipient, yet provides for a structure which is polar aprotic in nature. Suitable soluble grades of PVP as provided by BASF can includes Kollidon® grades K-12 (molecular weight range 2,000-3,000; pH 4.63), K-17 (molecular weight 7,000-11,000; pH 4.64), K-25 (molecular weight 28,000-34,000; pH 4.00), K-30 (molecular weight 44,000-54,000; pH 4.10), and K-90 (molecular weight 1,000,000-1,500,000; pH 5.68. Other functional polymers may include Kollidon® VA64 (molecular weight range 45,000-70,000, pH 4.51) or other povidones and copolymers thereof by different vendors. The present inventors have found that the use of polyvinylpyrrolidone in the presence of lenalidomide increases the solubility and stability of the lenalidomide.

The amount of the polyvinylpyrrolidone contained in the drug containing polymer layer can range from about 0.5 wt. % to about 50 wt. %, such as from about 0.75 wt. % to about 25 wt. %, such as from about 1 wt. % to about 10 wt. % based on the dry weight of the drug containing polymer layer.

c. Thickener

The drug containing polymer layer of the transdermal drug delivery system of the present invention can also include one or more thickening agents. The one or more thickening agents can include natural polymers, polysaccharides and their derivatives such as but not limited to agar, alginic acid and derivatives, cassia tora, collagen, gelatin, gellum gum, guar gum, pectin, potassium, or sodium carageenan, tragacanth, xantham, gum copal, chitosan, resin etc.), semisynthetic polymers and its derivatives such as without any limitation to cellulose and its derivatives (methylcellulose, ethyl cellulose, carboxymethyl cellulose, hydroxylpropyl cellulose (Klucel HF), hydroxylpropylmethyl cellulose, hydroxypropyl methylcellulose acetate succinate etc.), synthetic polymers and its derivatives such as without any limitation to carboxyvinyl polymers or carbomers (Carbopol® 940, Carbopol® 934, Carbopol® 971p NF), polyethylene and its copolymers, clays such as but not limited to silicates and bentonite, silicon dioxide, fumed silica (Aerosil®), polyvinyl alcohol, acrylic polymers (Eudragit®), acrylic acid esters, polyacrylate copolymers, polyacrylamide, polyvinyl pyrrolidone homopolymer and polyvinyl pyrrolidone copolymers such as but not limited to (PVP, Kollidon® 30, poloxamer), acrylic polymers such as but not limited to Eudragit® L100-55, Eudragit® RL, Eudragit® S-100, Eudragit® L-100, Plastoid® B, Eudragit® EPO, isobutylene, ethyl vinyl acetate copolymers, natural rubber, synthetic rubber, hot melt adhesives, styrene-butadiene copolymers, bentonite, all water and/or organic solvent swellable polymers, etc., or combinations thereof.

Regardless of the particular thickening agent utilized, the amount of the thickening agent contained in the drug containing polymer layer, if present, can range from about 0.1 wt. % to about 75 wt. %, such as from about 0.5 wt. % to about 50 wt. %, such as from about 0.75 wt. % to about 25 wt. % based on the dry weight of the drug containing polymer layer.

d. Skin Permeation Enhancer

The drug containing polymer layer of the transdermal drug delivery system of the present invention can also include one or more suitable, surfactants, plasticizers, humectants, or a combination thereof that can serve as a skin permeation enhancer to improve the permeation of the immunomodulatory agent through the skin during use of the transdermal drug delivery system. In one particular embodiment, the plasticizer can include various fatty alcohol, fatty acid, and or fatty ester derivatives, such as oleic acid, oleyl alcohol, ethyl oleate, oleyl oleate, polyethylene glycol ethers of oleyl alcohol, polyethylene glycol ethers of oleyl alcohol, linoleic acid, lauric acid, lauryl alcohol, lauryl lactate, myristic alcohol, isopropyl palmitate, etc. or humectants (glycerin, triethyl citrate, triacetin, glycols, diethylene glycol monoethyl ether, PEG, etc.), or a combination thereof. In one particular embodiment, the skin permeation enhancer can be a non-ionic surfactant that can include fatty derivatives of polyoxyethylene. One example is oleth-3, which is a polyethylene glycol ether of oleyl alcohol having three ethylene oxide units, although other oleths (e.g., −2, −4, −5, −6, −7, −8, −9, −10, −11, −12, −15, −16, −20, −23, −25, −30, −40, −44, and −50), are also contemplated either alone or in combinations thereof. Without intending to be limited by any particular theory, is believed that the oleth contributes to an increase in flux and the ability of the system to overcome a barrier of drop in flux 24-hours post application to the skin. Another non-ionic surfactant that is contemplated is a poloxamer (e.g., P181, P188, P338, P407, or a combination thereof, commercially available as Kolliphor®, Pluronic®, or Lutrol®). Still other non-ionic surfactants that can be utilized include laureths, ceteths, ceteareths, and steareths, either alone or combination with each other or with one or more of the oleths and/or poloxamers referenced above.

In one particular embodiment, the skin permeation enhancer can include a combination of a polyethylene glycol, methyl laurate, lauryl lactate, and a polyoxyethylene oleyl ether such as Brij® 010.

Regardless of the particular plasticizer, humectant, or combinations thereof utilized, the amount of the skin permeation enhancer in the drug containing polymer layer can range from about 1 wt. % to about 80 wt. %, such as from about 5 wt. % to about 60 wt. %, such as from about 10 wt. % to about 40 wt. % based on the dry weight of the drug containing polymer layer of the transdermal drug delivery system.

e. Skin Modifiers

The drug containing polymer layer of the transdermal drug delivery system of the present invention can also include one or more skin modifiers, fillers, protectants, antioxidants, other materials. Suitable skin or adhesive modifiers can include butylated hydroxytoluene (BHT), butylated hydroxyanisole (BHA), gallic acid, ascorbic acid, ascorbyl palmitate, lactic acid, methyl salicylate, salicylic acid or a combination thereof.

Regardless of the one or more skin modifiers, fillers, protectants, antioxidants, other materials contained in the drug containing polymer layer, such components can be present in the drug containing layer in a total amount ranging from about 0.5 wt. % to about 50 wt. %, such as from about 1 wt. % to about 25 wt. %, such as from about 1.5 wt. % to about 15 wt. % based on the dry weight of the drug containing polymer layer of the transdermal drug delivery system.

f. Polar Aprotic Solvent

The drug containing polymer layer can further include one or more polar aprotic solvents, which can be used to assist in the solubility of the immunomodulatory agent in the drug in the drug containing layer and in the delivery of the immunomodulatory agent through the skin. A polar aprotic solvent is a solvent that lacks an acidic proton and is polar. Such solvents lack hydroxyl and amine groups. These solvents do not serve as proton donors in hydrogen bonding, although they can be proton acceptors. Specific examples contemplated by the present invention can include n-methyl-2-pyrrolidone (NMP), dimethyl sulfoxide (DMSO), dimethyl isosorbide, ethyl acetate, or a combination thereof, although it is to be understood that other polar aprotic solvents are also contemplated by the present invention, including, but not limited to, acetone, acetonitrile, dichloromethane, dimethylformamide, DMPU, and tetrahydrofuran.

Regardless of the particular polar aprotic solvent or combination of polar aprotic solvents utilized, the total amount of polar aprotic solvent contained in a polymer blend used to form the drug containing polymer layer can be detectable in the transdermal drug delivery system in an amount less than ICH Q3C Impurities: Guideline for Residual Solvents. For NMP, this equates to levels of less than about 530 parts per million, or less than about 0.053 wt. %, such as less than about 390 parts per millions, or less than about 0.039 wt. %, based on the dry weight of the drug containing polymer layer where the NMP is to be considered a process solvent. However, it is to be understood that such solvents are introduced in larger wt. % levels during the formation of the drug containing polymer layer and prior to any evaporation or drying. Further, when utilized as an excipient, the one or more polar aprotic solvents can be present in an amount greater than the ICH Q3C Impurities: Guideline for Residual Solvents. For NMP, this equates to levels greater than about 390 parts per million, or greater than about 0.039 wt. %, such as greater than about 530 parts per million, or greater than about 0.053 wt. % based on the dry weight of the drug containing polymer layer.

In another embodiment, regardless of the particular polar aprotic solvent or combination of polar aprotic solvents utilized, the total amount of polar aprotic solvent contained in a polymer blend used to form the drug containing polymer layer can be detectable in the transdermal drug delivery system in an amount less than about 20,000 parts per million, or less than about 2.0 wt. %, such as less than about 10,000 parts per millions, or less than about 1.0 wt. %, based on the dry weight of the drug containing polymer layer. However, it is to be understood that such solvents are introduced in larger wt. % levels during the formation of drug containing polymer layer and prior to any evaporation or drying. Further, when utilized as an excipient, the one or more polar aprotic solvents can be present in an amount greater than about 530 parts per million, or greater than about 0.053 wt. %, such as greater than about 10,000 parts per million, or greater than about 1.0 wt. %, such as greater than about 20,000 parts per million, or greater than about 2 wt. %, based on the dry weight of the drug containing polymer layer.

### II. Backing Layer

Referring again to FIG. 3, in addition to the adhesive layer 305 and the drug containing polymer layer 310, the transdermal drug delivery system 300 of the present invention can include a backing layer 320 that forms the exterior surface 340 of the transdermal drug delivery system 300. The backing layer 320 can be occlusive in nature and can protect the polymer layer (and any other layers present) from the environment and prevents loss of the drug and/or release of other components to the environment during use. Materials suitable for use as backing layers are well-known known in the art and can comprise films of polyester, polyethylene, vinyl acetate resins, ethylene/vinyl acetate copolymers, polyvinyl chloride, polyurethane, and the like, metal foils, non-woven fabric, cloth and commercially available laminates. A typical backing material has a thickness in the range of 2 to 1000 micrometers. For example, 3M's Scotchpak® 1012 or 9732 (a polyester film with an ethylene vinyl acetate copolymer heat seal layer), 9723 (a laminate of polyethylene and polyester), 9754 (a polyester film backing laminate), or CoTran® 9720 (a polyethylene film) are useful in the transdermal drug delivery systems described herein, as are Dow® backing layer films, such as Dow® BLF 2050 (a multi-layer backing comprising ethylene vinyl acetate layers and an internal SARAN® layer.

### III. Release Liner

Referring still to FIG. 3, in addition the drug containing polymer layer 310, the backing layer 320, and the adhesive layer 305 disposed therebetween, the transdermal drug delivery system 300 of the present invention can also include a release liner 330 disposed on the skin-contacting surface 350 of the transdermal drug delivery system that protects the drug containing polymer layer 310 of the transdermal drug delivery system 300 until it is ready to be applied to a patient's skin. Once the transdermal drug delivery system 300 is to be applied to a patient's skin at its skin-contacting surface 350, the release liner 330 can be removed and discarded. Materials suitable for use as release liners are well-known known in the art and include the commercially available products of Dow Corning Corporation designated Bio-Release® liner and Syl-off® 7610, Loparex's PET release liner (silicone-coated), Saint Gobaine's 9011 liner, and 3M's 1020, 1022, 9741, 9744, 9748, 9749 and 9755 Scotchpak® liners, which are fluoropolymer-coated polyester films.

### IV. Method of Making the Transdermal Drug Delivery System

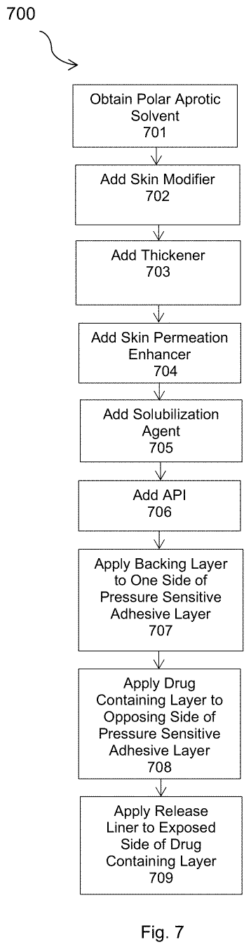

Generally, the transdermal drug delivery system having a non-drug containing layer and a drug containing layer disposed between a backing layer and a release liner is made by combining the components in a specific order, resulting in the ability to form a transdermal drug delivery system that exhibits controlled release immunomodulatory agent for an extended period of time and improved permeation of the immunomodulatory agent through the skin. Referring to FIG. 7, one method 700 of making a polymer blend used to form the solubilized drug in adhesive layer of the present invention is shown. First, in step 701, the API (e.g., an immunomodulatory agent) is obtained. Next, in step 702, the polar aprotic solvent is combined with a skin permeation enhancer. Then, in step 703, a skin modifier is added to the solution, followed by a solubilization agent, which can also be referred to as a crystallization inhibitor, in step 704, followed by mixing for about 5 minutes to about 1 hour. Next, in step 705, the immunomodulatory agent can be added and stirred for about 5 minutes to about 1 hour. Thereafter, in step 706, a thickener can be added and the solution stirred for about 12 to about 24 hours to solubilize the polymers, followed by sonication for about 15 to about 30 minutes to remove any air bubbles. Next, in step 707, a backing layer can be applied to a separate pressure sensitive adhesive, non-drug containing layer. Further, in step 708, the side of the non-drug containing layer not containing the backing layer can be coated with the drug containing later, after which a release liner can be applied to the opposing surface in step 709. Furthermore, the present method 700 contemplates that one or more components of the drug in polymer layer can be added in any order different from that described above so long as a homogenous, solubilized drug in polymer layer is formed prior to application to the adhesive, non-drug containing layer.

In any event, the present invention can also include a transdermal drug delivery system for administration of LLD that includes an active substance area including an immunomodulatory agent and at least one excipient; an impermeable backing layer; and optionally, a releasing membrane, which is covered by a detachable backing layer. The invention provides a transdermal drug delivery system where the active substance area or reservoir is configured as a polymer matrix system.

For instance, a transdermal drug delivery system is contemplated where the active substance matrix is constructed using water soluble polymers, which is then coated on the adhesive layer. Further, the active substance reservoir can be prepared as a polymer matrix. In addition, the active substance reservoir can be confined on the skin facing side of the transdermal drug delivery system by an active substance permeable membrane and on the opposite side from the skin by an active substance impermeable layer followed by adhesive layer.

The invention provides a transdermal drug delivery system comprising an active substance matrix containing area is a double or multilayered active substance matrix. In another embodiment, the active substance, LLD is in the simplest case dispersed, coarsely, colloidally or molecularly, in a solution or melt of base polymers. In the further a transdermal drug delivery system manufacturing techniques, the LLD is in the form of supersaturated solution, nano-emulsion or nano-suspension, amorphous, crystalline, co-crystals, coated with base polymers or solubilize in polymers using hot melt extrusion process.

The invention also includes such embodiments where the LLD matrix has a two or multi-layered structure, also called multi-laminate drug in adhesive patch. For example, the various matrix layers may contain polymer constitutes from the above-mentioned polymers. In this case, the matrix layers are differing from each other's in the term of polymer or pressure sensitive or hot melt polymers composition, LLD concentration, different permeating enhancers or solubilizers. The layers can be separated using semi-permeable membrane between two distinct drug-in-adhesive layers or multiple drug-in-adhesive layers under a single backing film. The term polymer film includes polymer without any limitation pressure sensitive adhesive and/or non-adhesive polymer.

In one aspect the invention further provides a polymer matrix formulation comprising LLD and a polymeric vehicle system. The vehicle system can include solvents (e.g., a solubilizer), permeability enhancing excipients and polymer or gelling agent or thickening agent, if required acid or base for pH adjustment.

### Pretreatment Composition and Method of Use

Various approaches have been used to open the barrier property of stratum corneum for drug permeation enhancement. Pretreatment with the use of chemical penetration enhancers is one of the techniques employed. The pretreatment has a potential to modulate the outermost layer of the skin reversibly and facilitate the drug uptake. Penetration enhancers act on lipid and protein regions in combination or alone on each region.

Penetration enhancers may be incorporated into the formulations described above (e.g., transdermal drug delivery systems including drug in adhesive layers and separate adhesive and drug containing layers), however, it can lead to some incompatibility or interactions within the ingredients. Therefore, the present invention includes the alternative method of skin penetration enhancement as to preparation/pretreatment the skin with some penetration enhancers or a combination of penetration enhancers before the patch application.

Pretreatment applications described herein include application of a gel/spray/solution/wetting agent/soaked swab/soaked cotton ball/soaked gauzes to the skin prior to application of drug containing product, intended to be a patch. However, it is to be understood that the pretreatment composition can include another topical dosage form, solution gel, cream, etc. For instance, the pretreatment composition can be its own individual patch, such as Curad Mediplast, a 40% salicylic acid patch, or a placebo patch comprising non-volatile components such as acrylic, silicone, or PIB adhesives or combinations thereof with an optional addition of a skin permeation enhancers to promote delivery of an active pharmaceutical ingredient through the skin.

The present invention provides a pretreatment composition wherein the penetration enhancers are incorporated in the form the topical dosage form as solution, gel, cream, spray, wetting agent, soaked cotton balls and gauzes. In yet another embodiment pretreatment composition preferably but not limited to gel can be incorporated in a reservoir patch.

### I. Pretreatment Composition

a. Polar Aprotic Solvent

The pretreatment composition of the transdermal drug delivery system of the present invention can further include one or more polar aprotic solvents, which can assist in the delivery of the immunomodulatory agent through the skin. A polar aprotic solvent is a solvent that lacks an acidic proton and is polar. Such solvents lack hydroxyl and amine groups. These solvents do not serve as proton donors in hydrogen bonding, although they can be proton acceptors. Specific examples contemplated by the present invention can include n-methyl-2-pyrrolidone (NMP), dimethyl sulfoxide (DMSO), dimethyl isosorbide, or a combination thereof, although it is to be understood that other polar aprotic solvents are also contemplated by the present invention, including, but not limited to, acetone, acetonitrile, dichloromethane, dimethylformamide, DMPU, and tetrahydrofuran.

Regardless of the particular polar aprotic solvent or combination of polar aprotic solvents utilized, the total amount of polar aprotic solvent contained in the pretreatment composition can range from about 25 wt. % to about 95 wt. %, such as from about 30 wt. % to about 50 wt. % to about 90 wt. %, such as from about 60 wt. % to about 80 wt. %. Further, when more than one polar aprotic solvent is present, n-methyl-2-pyrrolidone and dimethyl sulfoxide can be used, where the ratio of the n-methyl-2-pyrrolidone to the dimethyl sulfoxide can range from about 1.4:1 to about 2:1, such as from about 1.5:1 to about 1.9:1, such as from about 1.6:1 to about 1.8:1.

b. Humectant

The pretreatment composition of the transdermal drug delivery system of the present invention can further include one or more humectants that act as a carrier. Specific examples contemplated by the present invention can include glycerin, polyglycol, and polyethylene glycol (e.g., PEG 400 or other molecular weights), triethyl citrate, triacetin, etc.

Regardless of the particular humectant utilized, the total amount of humectant in the pretreatment composition can range from about 1 wt. % to about 80 wt. %, such as from about 2 wt. % to about 25 wt. %, such as from about 3 wt. % to about 20 wt. %.

c. Weak Organic Acid/More than 1 Carbon Chain Containing Acid

The pretreatment composition of the transdermal drug delivery system of the present invention can further include one or more weak organic acids or more than 1 carbon chain containing acid. Specific examples contemplated by the present invention can include levulinic acid, oleic acid, lactic acid, salicylic acid, or a combination thereof.

Regardless of the particular acid utilized, the total amount of acid in the pretreatment composition can range from about 1 wt. % to about 40 wt. %, such as from about 2 wt. % to about 35 wt. %, such as from about 3 wt. % to about 30 wt. %. Further, when salicylic acid is utilized, it has been surprisingly found that the presence of the salicylic acid in an amount less than 10 wt. %, such as from about 1 wt. % to about 7 wt. %, such as from about 1.5 wt. % to about 6 wt. %, such as from about 2 wt. % to about 5 wt. %, results in increased flux compared to concentrations of salicylic acid of 10 wt. % or greater.

d. Thickener

The pretreatment composition of the transdermal drug delivery system of the present invention can also include one or more thickening agents. The one or more thickening agents can include natural polymers, polysaccharides and their derivatives such as but not limited to agar, alginic acid and derivatives, cassia tora, collagen, gelatin, gellum gum, guar gum, pectin, potassium, or sodium carageenan, tragacanth, xantham, gum copal, chitosan, resin etc., semisynthetic polymers and its derivatives such as without any limitation to cellulose and its derivatives (methylcellulose, ethyl cellulose, carboxymethyl cellulose, hydroxylpropyl cellulose (Klucel HF), hydroxylpropylmethyl cellulose, hydroxypropyl methylcellulose acetate succinate etc.), synthetic polymers and its derivatives such as without any limitation to carboxyvinyl polymers or carbomers (Carbopol® 940, Carbopol® 934, Carbopol® 971p NF), polyethylene and its copolymers, clays such as but not limited to silicates and bentonite, silicon dioxide, fumed silica (Aerosil®), polyvinyl alcohol, acrylic polymers (Eudragit®), acrylic acid esters, polyacrylate copolymers, polyacrylamide, polyvinyl pyrrolidone homopolymer and polyvinyl pyrrolidone copolymers such as but not limited to (PVP, Kollidon® 30, poloxamer), isobutylene, ethyl vinyl acetate copolymers, natural rubber, synthetic rubber, hot melt adhesives, styrene-butadiene copolymers, bentonite, all water and/or organic solvent swellable polymers, etc. or combinations thereof.

Regardless of the particular thickening agent utilized, the amount of the thickening agent contained in the pretreatment composition, if present, can range from about 0.1 wt. % to about 30 wt. %, such as from about 0.5 wt. % to about 20 wt. %, such as from about 0.75 wt. % to about 10 wt. % based on the weight of the pretreatment composition.

e. Volatile Carrier Solvent

The pretreatment composition of the transdermal drug delivery system of the present invention can further include one or more volatile carrier solvents. Specific examples contemplated by the present invention can include water, ethanol, isopropyl alcohol, and similar solvents.

Regardless of the particular volatile carrier solvent that may be utilized, the total amount of humectant in the pretreatment composition can range from about 1 wt. % to about 99 wt. %, such as from about 2 wt. % to about 98 wt. %, such as from about 3 wt. % to about 97 wt. %.

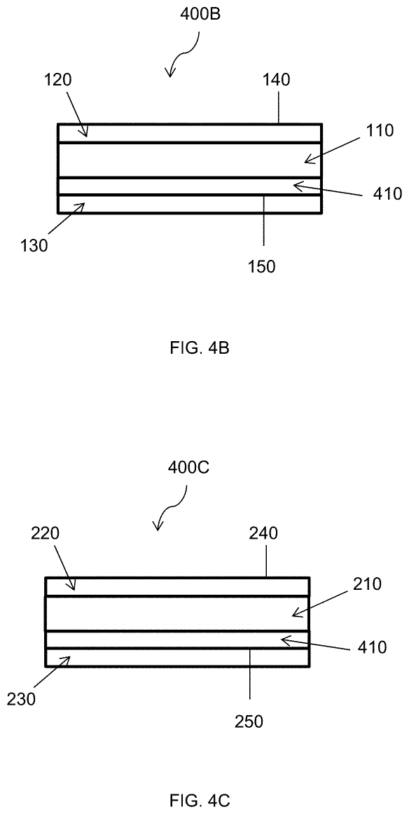

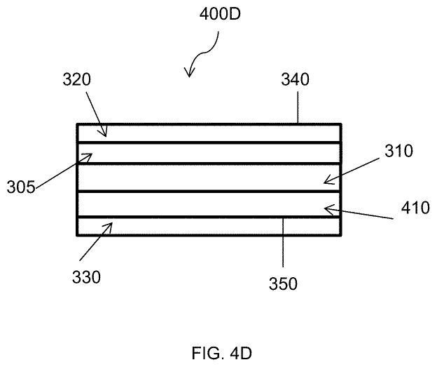

Referring to FIG. 4A, in one embodiment, the pretreatment composition 410 can be part of a kit 400A that also includes a transdermal drug delivery system 100, 200, or 300 in the form of a patch and described in detail above.

In another embodiment and referring to FIG. 4B, the pretreatment composition 410 can be part of the transdermal drug delivery system 400B that includes a solubilized drug in adhesive layer 110 disposed between a backing layer 120 and a release liner 130, where the pretreatment layer 410 is positioned between the solubilized drug in adhesive layer 110 and the release liner 130. The backing layer 120 has an exterior surface 140 that is exposed to the ambient environment when the transdermal drug delivery system 400B is in use.

Meanwhile, the release liner 130 is positioned on a skin-contacting surface 150 of the solubilized drug in adhesive matrix layer 110, where the release liner 130 is removable so that the pretreatment composition 410 can be positioned directly on the skin during use of the transdermal drug delivery system 400B.

In still another embodiment and referring to FIG. 4C, the pretreatment composition 410 can be part of a transdermal drug delivery system 400C that includes a solid dispersion drug in adhesive layer 210 disposed between a backing layer 220 and a release liner 230, where the pretreatment layer 410 is positioned between the solid dispersion drug in adhesive layer 210 and the release liner 230. The backing layer 220 has an exterior surface 240 that is exposed to the ambient environment when the transdermal drug delivery system 400B is in use.

Meanwhile, the release liner 230 is positioned on a skin-contacting surface 250 of the solid dispersion drug in adhesive layer 210, where the release liner 230 is removable so that the pretreatment composition 410 can be positioned directly on the skin during use of the transdermal drug delivery system 400C.

In yet another embodiment and referring to FIG. 4D, the pretreatment composition 410 can be part of a transdermal drug delivery system 400D that includes a drug containing polymer layer 310 disposed between a backing layer 320 and a release liner 330, where the pretreatment layer 410 is positioned between the drug containing polymer layer 310 and the release liner 230. Further, a separate adhesive layer 305 is disposed between the drug containing polymer layer 310 and the backing layer 320. The backing layer 320 has an exterior surface 340 that is exposed to the ambient environment when the transdermal drug delivery system 400D is in use. Meanwhile, the release liner 330 is positioned on a skin-contacting surface 350 of the drug containing polymer layer 310, where the release liner 330 is removable so that the pretreatment composition 410 can be positioned directly on the skin during use of the transdermal drug delivery system 400D.

### II. Method of Use

In one embodiment, the pretreatment composition described above can be designed to be applied for about 1 minute up to about 72 hours, such as from about 30 minutes to about 10 hours, such as from about 1 hour to about 5 hours prior to application or contact of any drug containing layer of the transdermal drug delivery systems described above. The invention contemplates a pretreatment skin composition in the application dose of about 10 mg/cm² to about 1000 mg/cm², such as from about 100 mg/cm² to about 800 mg/cm².

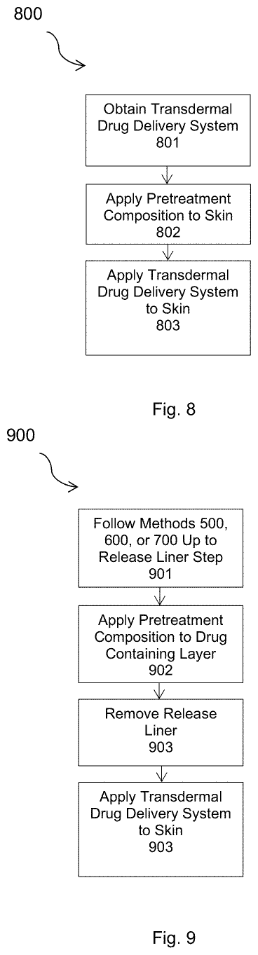

For instance, and referring to FIG. 4A and FIG. 8, the pretreatment composition 410 can be part of a kit or a stand alone composition. In any event, one method 800 of using the pretreatment composition can include obtaining a transdermal drug delivery system in step 801, applying the pretreatment composition to a surface of skin in step 802, and then applying the transdermal drug delivery system to the skin in step 803 after a predetermined amount of time has passed (e.g., about 1 minute to about 72 hours). It should also be understood that the pretreatment composition can be removed from the skin, after which the transdermal drug delivery system can be immediately applied. In other embodiments, at least part of the pretreatment composition may evaporate or leave little to no residue on the patient's skin such that removal is not necessary.

Alternatively, and referring to FIGS. 4B-4D, 5-7 and 9, another method 900 can include following the method steps set forth in method 500, 600, or 700 up to the release liner step in step 901 to form the contemplated transdermal drug delivery systems, applying the pretreatment composition to a drug containing layer in step 902, applying a release liner to the pretreatment composition 410 in step 903, removing the release liner once the system is ready to be applied to the skin in step 904, and applying the transdermal drug delivery system to the skin in step 905. It should also be understood that the entire transdermal drug delivery system including the portion containing the pretreatment composition 410 can be removed from the skin, whereafter the pretreatment composition 410 can be peeled away from a barrier liner (not shown) that can be disposed between the pretreatment composition 410 and the solubilized drug in adhesive layer 110, the solid dispersion drug in adhesive layer 210, or the drug containing layer 310, after which the transdermal drug delivery system can be immediately applied to the skin such that the solubilized drug in adhesive layer 110, the solid dispersion drug in adhesive layer 210, or the drug containing layer 310 is applied directly to the skin.

In any event, the invention provides a pretreatment composition and/or a single component that can include skin permeation-enhancing agents one or more such as but not limited to water, sulfoxides, and similar chemicals such as but not limited to dimethyl sulfoxide, dimethylacetamide, dimethylformamide, decylmethylsulfoxide, dimethyl isosorbide etc.; azone, pyrrolidones such as but not limited to n-methyl-2-pyrrolidone, 2-pyrrolidone etc.; esters such as but not limited to propylene glycol monolaurate, butyl ethanoate, ethyl ethanoate, isopropyl myristate, isopropyl palmitate, methyl ethanoate, decyl oleate, glycerol monooleate, glycerol monolaurate, lauryl laurate, methyl laurate, etc.; fatty acids (C3 and above) such as but not limited to lactic acid, salicylic acid, capric acid, caprylic acid, lauric acid, oleic acid, myristic acid, linoleic acid, stearic acid, palmitic acid, etc.; Brij® (such as but not limited to Brij® 05, Brij® 010, 03); alcohols, fatty alcohols and glycols such as but not limited to oleyl alcohol, ethanal, dodecanol, polyethylene glycol, propylene glycol, glycerol etc.; volatile chemicals such as ethanol, isopropyl alcohol; ethers such as but not limited to diethylene glycol monoethyl ether; urea, polyoxyethylene fatty alcohol ethers, polyoxyethylene fatty acid esters, esters of fatty alcohols, esters of long chain fatty acids with methyl, ethyl or isopropyl alcohol, esters of fatty alcohols with acetic acid, lactic acid, as well as oleic acid, diethanolamine, essential oils, terpene and terpenoids such as but not limited to erpineol, limonene, thymol, cineole, etc.; surfactant type enhancers such as polysorbate 80, polysorbate 20, etc.; liposomes, niosomes, transferomes, ethanosomes, etc. and all penetration or permeation enhancers referred in the book “Percutaneous Penetration Enhancers” (Eric W. Smith, Howard I. Mailbach, 2005. November, CRC press). The permeation-enhancing substances mentioned above may be added either singly or as a mixture.

The present invention may be better understood by reference to the following examples.

### Example 1

Example 1 focuses on the development of a stable solubilized drug in adhesive formulation.

Solubility of Lenalidomide H1 was conducted in more than 50 solvents and polymers. Only four solvents have more than 5% solubility of Lenalidomide. More than 20% solubility of lenalidomide was observed in two polar aprotic solvents N-methyl-2-pyrrolidone (NMP) and dimethyl sulfoxide followed by more than 5% solubility in two organic acids that is Lactic acid and levulinic acid. Lenalidomide has less than 2% solubility in glycols and less than 0.5% solubility in esters. Lenalidomide has about 1.5% to about 2% solubility in a Kollidon (polyvinylpyrrolidone) and solvent system (Methanol: Acetone). Lenalidomide has poor solubility in most commonly used solvents, and it is difficult to solubilize lenalidomide.

TABLE 1
Lenalidomide H1 solubility in polar aprotic solvents

| Solvent | Solubility % W/W |
|---|---|
| NMP | 29.3 |
| Dimethyl sulfoxide | 20.7 |
| Acetonitrile | 0.3 |
| Acetone | 0.2 |

TABLE 2
Lenalidomide H1 solubility in acid

| Solvent | Solubility % W/W |
|---|---|
| Lactic acid | 9.3 |
| Levulinic acid | 5.5 |
| Isooctadecanoic acid | <0.1 |

TABLE 3
Lenalidomide H1 solubility in Alcohols

| Solvent | Solubility % W/W |
|---|---|
| Super refined PEG 400 | 1.6 |
| Propylene Glycol | 0.4 |
| Methanol | 0.3 |
| Ethanol 190 Proof | 0.2 |

TABLE 4
Lenalidomide H1 solubility in esters

| Solvent | Solubility % W/W |
|---|---|
| Lauryl Lactate | 0.1 |
| Methyl Laurate | <0.1 |
| Propylene Glycol monolaurate | <0.1 |

TABLE 5
Lenalidomide H1 solubility in other solvents

| Solvent | Solubility % W/W |
|---|---|
| Toluene | <0.1 |
| Ethyl acetate | <0.1 |
| Heptane | <0.1 |

TABLE 6
Lenalidomide H1 solubility in Polymers using solvent system
Acetone:Methanol (1:1)

| | Solubility |
|---|---|
| Solvent | % W/W |
| Methanol:Acetone (1:1) | 0.9 |
| Kollidon 30 (0.5 G) + Solvent system | 2.2 |
| Kollidon 90 F (0.5 G) + Solvent system | 2.0 |
| Kollidon VA 64 (0.5 G) + Solvent system | 2.1 |
| Aquasolve HPMC-AS MF (0.25 G) + Solvent system | 1.2 |
| Eudragit + Solvent system | 0.9 |
| Kollisolv P124 Geismer (0.27 G ) + Solvent system | 0.9 |

In vitro permeability of lenalidomide through human cadaver skin was conducted using in vitro franz diffusion cells to identify potential permeation enhancers for lenalidomide. Donor compartment was loaded with Lenalidomide solution or gel.

### Solution Formulation

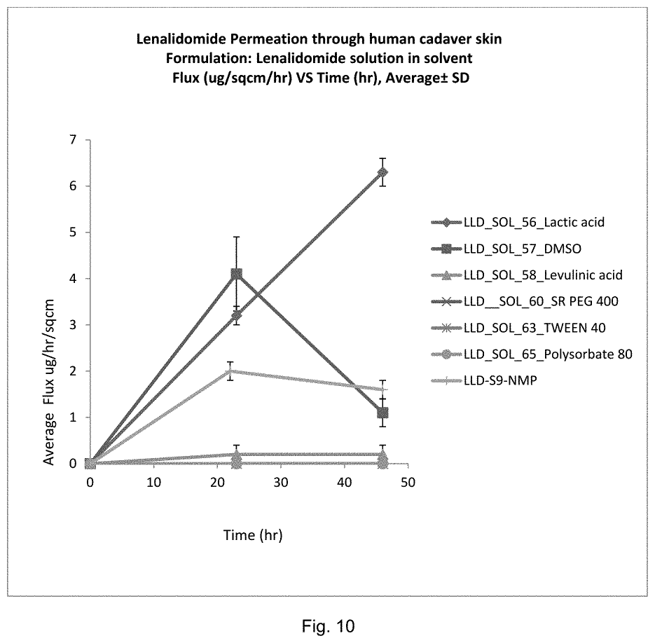

Lenalidomide H1 solutions were prepared in following different solvents Lactic acid, Dimethyl sulfoxide, NMP, Levulinic acid, Super refined PEG 400, Tween 40, Polysorbate 80. As shown in FIG. 10, the highest rate of lenalidomide permeation was observed from Lactic acid, followed by NMP and DMSO, while negligible permeation was observed from Levulinic acid, and there was insignificant to no permeation from SR PEG 400, TWEEN 40, and Polysorbate 80. Therefore, it appears Lactic acid, NMP, and DMSO are permeation enhancers for lenalidomide and facilitate its permeability through the skin, as shown in FIG. 10.

### Gel Formulation

Lenalidomide gels were prepared using a following solvent system composition (Table 7). Gels were made with 10 different permeation enhancers.

TABLE 7
Lenalidomide Gel composition

| Ingredients | % W/W (LLDG_001) | % W/W |
|---|---|---|
| Lenalidomide H1 | 6.6 | 6 |
| Dimethyl sulfoxide | 47.3 | 43 |
| NMP | 27.5 | 25 |
| Super refined PEG 400 | 17.6 | 16 |
| Klucel HF Pharm (Cellulose ether) | 1.1 | 1 |
| Permeation Enhancer* | — | 9 |

*Permeation Enhancer: Lactic acid, Oleic acid, Oleyl alcohol, glyceryl monooleate(GMO), methyl laurate, lauryl lactate, triacetin, Brij ® O3, Brij ® O5, Brij ® O10

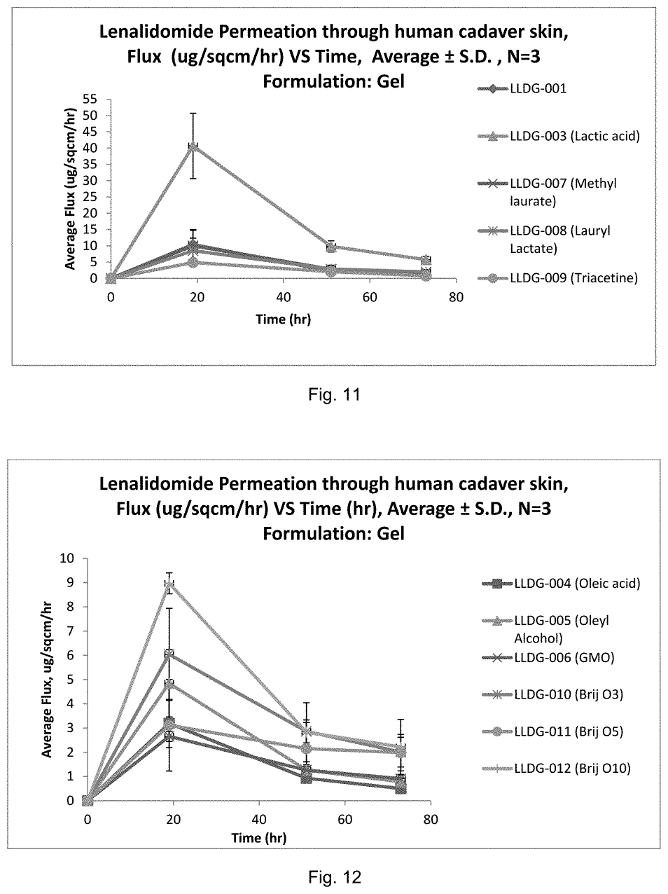

As shown in FIGS. 11-12, the highest permeation of lenalidomide was observed in gel containing Lactic acid, followed by Methyl laurate, Lauryl lactate and Brij O10. On the other hand, compared to LLDG_001, gels with Oleic acid, Oleyl alcohol, Glyceryl monooleate (GMO), Triacetin, Brij O3 and Brij O5 did not improve LLD permeation. Surprisingly, very high permeation of lenalidomide was observed from gel containing Lactic acid.

Next, the solubility of lenalidomide H1 was determined in silicone polymer-based pressure sensitive adhesives and acrylic polymer-based pressure sensitive adhesives. Pressure sensitive adhesive polymers are the major component of the pressure sensitive adhesives matrix patches and typically constitute 50%-85% of the formulation. Due to the poor solubility of lenalidomide in the pressure sensitive adhesives shown in Table 8 below, it became a challenge to prepare a soluble adhesive matrix patch of lenalidomide.

TABLE 8
Solubility of Lenalidomide in Various Pressure Sensitive Adhesives

| | Pressure sensitive | Lenalidomide H1 |
|---|---|---|
| | Adhesive | solubility % W/W |
| Acrylic polymer based | Duro-Tak 87-2074 | Less than 0.5% |
| pressure sensitive adhesives | Duro-Tak ® 87-2852 | Less than 0.5% |
| | Duro-Tak ® 87-9301 | Less than 0.5% |
| | Duro-Tak ® 87-2516 | approximately 0.5% |
| | Duro-Tak ® 87-2194 | Less than 0.5% |
| | Duro-Tak ® 87-4098 | Less than 0.5% |
| | Duro-Tak ® 87-2052 | Less than 0.5% |
| Silicone pressure sensitive | BIO-PSA ® 7-4202 | Less than 0.5% |
| adhesives | | |

As explained and shown below in Table 9, the addition of povidone (PVP) and NMP helps in solubilizing lenalidomide hemihydrate in adhesive matrix formulations. Examples of povidone include Kollidon 30 LP and Kollidon VA 64.

### NMP Improves Solubility of Lenalidomide in Adhesive Matrix Formulation

NMP is added to the formulation blend but majority of it evaporates while drying coated laminate in oven. More than 70% NMP is lost during drying, more preferably more than 80% NMP is lost during drying. NMP may be present in the dried laminate in the range of about 0.04%-2%. Due to high loss of NMP during drying NMP should be treated as process solvent at this stage of solubilized adhesive matrix patch formulation.

LLD MT 193: LLD MT 193 does not contain NMP and is not able to completely solubilize 2% Lenalidomide in blend. Blend has undissolved particles which is an indication of presence of insoluble lenalidomide.

LLD MT 187: LLD MT 187 contains NMP and is able to solubilize 3% Lenalidomide in blend. LLD MT 187 illustrates importance of NMP in solubilizing lenalidomide in adhesive matrix formulation blend.

### PVP Improves Solubility of Lenalidomide in Adhesive Matrix Formulation

LLD MT 192: LLD MT 192 contains NMP but does not contain PVP and is not able to solubilize 4% Lenalidomide in blend. The formulation blend is whitish in appearance, which is an indication that lenalidomide is not fully soluble in the blend.

LLD MT 165 and LLD MT 169: LLD MT 165 and LLD MT 169 contain both NMP and PVP and is able to solubilize 4% Lenalidomide in blend. Blend of both formulations are translucent in appearance. LLD MT 165 and 169 illustrate that PVP helps in solubilizing lenalidomide in adhesive matrix formulation blend.

TABLE 9
Solubility of Various LLD Formulations

| | LLD MT | LLD MT | LLD MT | LLD MT | LLD MT |
|---|---|---|---|---|---|
| | 193 | 187 | 192 | 165 | 169 |
| | (% w/w) | (% w/w) | (% w/w) | (% w/w) | (% w/w) |
| Lenalidomide | 2 | 3 | 4 | 4 | 4 |
| Hemihydrate | | | | | |
| Oleic acid | 16 | 16 | 16 | 16 | 16 |
| Isopropyl | 10 | 10 | 10 | 10 | 10 |
| Palmitate | | | | | |
| Ethylcellulose | 10 | 10 | 10 | 10 | 10 |
| N50 | | | | | |
| Kollidon ® 30 LP | — | — | — | 10 | — |
| Kollidon ® VA 64 | — | — | — | — | 10 |
| Dur-Tak ® | 52 | 52 | 52 | 52 | 52 |
| 387-2516 | | | | | |
| Total | 90 | 91 | 92 | 102 | 102 |
| NMP | — | Process | Process | Process | Process |
| | | solvent | solvent | solvent | solvent |
| Ethyl acetate | Process | Process | Process | Process | Process |
| | solvent | solvent | solvent | solvent | solvent |
| Blend | Undissolved | Translucent, | White | Translucent, | Translucent, |
| observation after | particles | homogenous, | homogenous | homogenous, | homogenous, |
| overnight mixing | | pale yellow | mixture | pale yellow | pale yellow |

Next, lenalidomide adhesive matrix patches were prepared using different combinations of excipients which includes povidone, adhesive polymers, cellulose polymers, commonly known permeation enhancers.

Adhesive matrix patch release study was conducted to understand impact of excipients on release of lenalidomide from patch. A release study was conducted using a roller. Lenalidomide adhesive matrix patches were added to glass scintillation vial containing media. Each patch in a scintillation vial was mixed on a roller for about 20-24 hr. At the end of study, an aliquot from each scintillation vial was collected and analyzed on HPLC to determine the amount of lenalidomide released from the patch. The results are shown and discussed below with reference to Tables 10 and 11 for various pressure sensitive adhesives.

### Release Study: Lenalidomide Adhesive Matrix Patch Containing PSA Duro-Tak® 387-2516 (Table 10)

LLD MT 9, LLD MT 60, LLD MT 65: 1 ug/sqcm/hr lenalidomide is released from patches containing Duro-Tak® 387-2516 with or without povidone. Polymers are holding up lenalidomide in matrix patch and retarding its release rate.

LLD MT 128: Almost four fold increase in lenalidomide was observed with LLDMT 128 compared to LLDMT 9, LLD MT 60 and LLD MT 65. Addition of Oleic acid, Isopropyl palmitate and Ethyl cellulose N50 helped in releasing lenalidomide from adhesive matrix patch. This is a significant improvement in release of lenalidomide from an adhesive matrix patch.

TABLE 10
Lenalidomide adhesive matrix patch containing PSA
Duro-Tak ® 387-2516

| | % W/W on dry basis | | | | |
|---|---|---|---|---|---|
| | LLD | LLD | LLD | LLD | LLD |
| Ingredients | MT 9 | MT 60 | MT 65 | MT 85 | MT 128 |
| Lenalidomide H1 | 2.5 | 2 | 2 | 2 | — |
| Lenalidomide | — | — | — | — | 2 |
| Hemihydrate | | | | | |
| Oleic acid | — | — | — | — | 16 |
| Isopropyl Palmitate | — | — | — | — | 10 |
| Ethyl Cellulose N50 | — | — | — | — | 10 |
| Kollidon ® 30 LP | — | — | 10 | — | 10 |
| Kollidon ® VA 64 | — | 10 | — | 10 | — |
| Duro-Tak ® | 97.5 | 88 | 88 | 44 | 52 |
| 387-2516 | | | | | |
| BIO PSA ® 4201 | — | — | — | 44 | — |
| NMP | Process | Process | Process | Process | Process |
| | solvent | solvent | solvent | solvent | solvent |
| Ethyl Acetate | — | — | — | — | Process |
| | | | | | solvent |
| Amount of | 1.1 | 0.8* | 0.8* | 0.6** | 3.9** |
| lenalidomide | | (2.4%) | (1.3%) | (0.9%) | (2.7%) |
| released in 24 hr, | | | | | |
| ug/sqcm/hr (% RSD) | | | | | |

*Average Amount of lenalidomide released in 20.5 hr, ug/sqcm/hr (% RSD), n = 3
**Average Amount of lenalidomide released in 23 hr, ug/sqcm/hr (% RSD), n = 3

### Release Study: Lenalidomide Adhesive Matrix Patch Containing PSA Duro-Tak® 87-4098 (Table 11)

LLD MT 16, LLD MT 52, LLD MT 113: less than 1 ug/sqcm/hr lenalidomide is released from patches containing Duro-Tak® 87-4098 with or without povidone.

LLD MT 127: Almost six fold increase in lenalidomide was observed with LLDMT 127 compared to LLDMT 52. Addition of Oleic acid, Isopropyl palmitate and Ethyl cellulose N50 helped in releasing lenalidomide from adhesive matrix patch. This is a significant improve in release of lenalidomide from an adhesive matrix patch.

TABLE 11
Lenalidomide adhesive matrix patch containing PSA
Duro-Tak ® 87-4098

| | % W/W dry basis | | | |
|---|---|---|---|---|
| | LLD MT | LLD MT | LLD MT | LLD MT |
| Ingredients | 16 | 52 | 113 | 127 |
| Lenalidomide H1 | 1.25 | 2 | — | — |
| Lenalidomide | — | — | 2 | 2 |
| Hemihydrate | | | | |
| Oleic acid | — | — | — | 16 |
| Isopropyl | — | — | — | 10 |
| Palmitate | | | | |
| Ethyl Cellulose | — | — | — | 10 |
| N50 | | | | |
| Kollidon ® 30 LP | — | — | 10 | 10 |
| Kollidon ® VA 64 | — | 10 | — | — |
| Duro-Tak ® | 97.5 | 88 | 88 | 52 |
| 87-4098 | | | | |
| NMP | Process | Process | Process | Process |
| | solvent | solvent | solvent | solvent |
| Ethyl Acetate | — | — | — | Process |
| | | | | solvent |
| Amount of | 0.33 | 0.8* (13%) | 0.35**(2.8%) | 4.81 **(2%) |
| lenalidomide | | | | |
| released in 24 hr, | | | | |
| ug/sqcm/hr | | | | |
| (% RSD) | | | | |

*Average Amount of lenalidomide released in 20.5 hr, ug/sqcm/hr (% RSD), n = 3
**A verage Amount of lenalidomide released in 23 hr, ug/sqcm/hr (% RSD), n = 3

Next, lenalidomide adhesive matrix formulation blends were prepared by keeping all of the excipients the same (oleic acid, isopropyl palmitate, ethyl cellulose n50, Kollidon 30 LP) and changing the pressure sensitive adhesive polymer in each formulation. Different pressure sensitive adhesive formulations were tried including acrylic PSA polymers (Duro-Tak®387-2516, Duro-Tak® 87-9301, Duro-Tak® 87-4098, Duro-Tak®87-2194, Duro-Tak®87-2052) and polyisobutylene (Duro-Tak®6908). However, the resulting blend was not homogenous for Duro-Tak® 87-9301, Duro-Tak® 87-4908, or Duro-Tak® 87-6908. It is a possibility that particulate in the blends (LLD MT 126, LLD MT 127) could be of lenalidomide or povidone. The broken blend (LLD MT 130) is an indication of immiscibility of excipients. Adhesives which formed a homogenous and translucent blends include LLDMT 128 (acrylate copolymer adhesive Duro-Tak® 387-2516) and LLD MT 131 (acrylate copolymer adhesive Duro-Tak® 87-2052).

TABLE 12
Solubility of LLD in Various Pressure Sensitive Adhesive Polymers

| | % W/W on dry basis | | | | | |
|---|---|---|---|---|---|---|
| | LLD MT | LLD MT | LLD MT | LLD | LLD MT | LLD MT |
| Ingredients | 126 | 127 | 128 | MT 129 | 130 | 131 |
| Lenalidomide | 2 | 2 | 2 | 2 | 2 | 2 |
| Hemihydrate | | | | | | |
| Oleic acid | 16 | 16 | 16 | 16 | 16 | 16 |
| Isopropyl | 10 | 10 | 10 | 10 | 10 | 10 |
| Palmitate | | | | | | |
| Ethyl | 10 | 10 | 10 | 10 | 10 | 10 |
| Cellulose | | | | | | |
| N50 | | | | | | |
| Kollidon 30 | 10 | 10 | 10 | 10 | 10 | 10 |
| LP | | | | | | |
| Duro-Tak ® | — | — | 52 | — | — | — |
| 387-2516 | | | | | | |
| Duro-Tak ® | 52 | — | — | — | — | — |
| 87-9301 | | | | | | |
| Duro-Tak ® | — | 52 | — | — | — | — |
| 87-4098 | | | | | | |
| Duro-Tak ® | — | — | — | 52 | — | — |
| 87-6908 | | | | | | |
| Duro-Tak ® | — | — | — | — | 52 | — |
| 87-2194 | | | | | | |
| Duro-Tak ® | — | — | — | — | — | 52 |
| 87-2052 | | | | | | |
| NMP | Process | Process | Process | Process | Process | Process |
| | solvent | solvent | solvent | solvent | solvent | solvent |
| Ethyl Acetate | Process | Process | Process | Process | Process | Process |
| | solvent | solvent | solvent | solvent | solvent | solvent |
| Blend | Broken, | Broken, | Yellow, | Broken | White, | Almost |
| observation | white, | white, | translucent, | | homogenously | transparent, |
| after | dispersed | dispersed | homogenous | | dispersed | homogenous |
| overnight | particles | particles | | | | |
| mixing | | | | | | |

Further, in vitro permeability from three different lenalidomide adhesive matrix patches (formulations shown in Table 13) through human cadaver skin was conducted using in vitro franz diffusion cells. Human cadaver skin was pretreated with PT 001 pretreatment gel for about 1 hour in franz diffusion cells. After about 1 hr PT 001 (formulation shown in Table 14) was gently wiped and removed from skin. Then, lenalidomide adhesive matrix patches applied to wiped skin and amount of lenalidomide permeated was quantified, as shown in FIG. 4.

TABLE 13
Lenalidomide adhesive matrix patch formulation composition

| | % W/W on dry basis | | |
|---|---|---|---|
| Ingredients | LLD MT 215 | LLD MT 204 | LLD MT 168 |
| Lenalidomide | 6.7 | 6.7 | 3 |
| hemihydrate | | | |
| Oleic acid | 15.3 | 15.3 | 15.8 |
| Isopropyl Palmitate | — | 4.8 | 9.9 |
| Ethyl cellulose n50 | 9.5 | 9.5 | 9.9 |
| Kollidon ® 30 LP | 9.5 | 9.5 | — |
| Kollidon ® VA 64 | — | — | 9.9 |
| Duro-Tak ® 387-2516 | 59 | 54.2 | 51.5 |
| Total | 100 | 100 | 100 |
| NMP | Process | Process | Process |
| | solvent | solvent | solvent |
| Ethyl Acetate | Process | Process | Process |
| | solvent | solvent | solvent |

TABLE 14
Pretreatment Formulation

| Ingredients | PT 001 (% W/W) |
|---|---|
| Dimethyl sulfoxide | 45.7 |
| NMP | 26.6 |
| Super refined PEG 400 | 17 |
| Lactic acid (Racemic) | 9.6 |
| Klucel HF Pharm | 1.1 |
| Total | 100 |

### Example 2

Example 2 focuses on the development of a stable solid drug dispersion in adhesive formulation.

Significant efforts were pursued to understand solubility of lenalidomide in various organic solvents and excipients typically used in pharmaceutical drug products, and specifically, in transdermal and topical formulations. It was found that NMP has significant affinity for the drug which exceeds all other tested materials by a factor of more than 2-fold the observed solubility at room temperature. Where, NMP can solubility up to about 30% w/w lenalidomide and the next closest excipient DMSO, can dissolve up to about 10-20%. Of particular interest is in the polar aprotic solvents. In particular is the structural similarity between NMP to lenalidomide where NMP structure closely resembles the heart of lenalidomide.

As a solubilized drug platform was already under consideration, formulations were pursued to include a pressure sensitive adhesive (PSA) platform formulation containing the PSA with drug and excipients to prepare a dispersed particulate, solid dispersion, micro-dispersion or other conceptual design in formulation platform to suspend the API at crystalline and or molecular level within a formulation as solid solution or solid suspension within an adhesive matrix. The formulation strategy was to prepare a mono-layer drug-in-adhesive system between a backing layer and a disposable release liner. Surprisingly, it was found that the addition of drug in a solubilized form with n-methyl-2-pyrrolidone (NMP) was necessary to incorporate the drug into a solution for addition and to produce a consistent and uniform polymer blend and resulting laminate after evaporation of process solvents including n-methyl-2-pyrrolidone (NMP).

A micronized grade of crospovidone, a cross-linked povidone (PVP), was incorporated to allow molecular adsorption onto a solid porous substrate. Other substrates may be viable such that they disperse within matrix and allow for affinity of crystalline and or molecular dispersion of API.

Initial formulation strategy was initiated to incorporate lenalidomide into the drug-in-adhesive solid dispersion-type formulation.

Initial formulations, as shown in Formulation Table 15, were made to assess dispersions with an anti-solvent composition in polyisobutylene and a heptane solvent system as heptane is a known anti-solvent for lenalidomide.

TABLE 15
Initial Testing of Solid Drug Dispersion in Adhesive Formulations

| Excipients | RDNB-0003-2-1 | RDNB-0003-2-2 |
|---|---|---|
| NMP | 12.6% | 4.0% |
| LLD | 1.0% | 1.0% |
| Kollidon ® CLM | 6.4% | 5.0% |
| Duro-Tak ® 87-6908 | 80% | 90% |
| Ethyl Acetate as | — | 2 × Kollidon CLM |
| process solvent | | |
| Observations | Formula Cracked and | Formulation was true |
| | Phase separated | dispersion |
| | No good | |

Due to the polarity of the NMP composition in formulation (3-2-1) above, it was difficult to obtain a uniform and consistent polymer blend. Formulation (3-2-2) above was able to produce a uniform blend and laminate and pursued further, with additional formulations shown in Table 16 below.

TABLE 16
Additional Testing of Solid Drug Dispersion in Adhesive Formulations

| Excipients | RDNB-0003-3-1 | RDNB-0003-3-2 | RDNB-0003-3-3 |
|---|---|---|---|
| NMP | 4.0% | 6.0% | 8.0% |
| LLD | 1.0% | 1.0% | 1.0% |
| Kollidon ® | 5.0% | 5.0% | 5.0% |
| CLM | | | |
| Duro-Tak ® | 90% | 88% | 86% |
| 87-6908 | | | |
| Ethyl Acetate | 2 × Kollidon | 2 × Kollidon | 2 × Kollidon |
| as process | CLM | CLM | CLM |
| solvent | | | |
| Observations | Dispersion with | Dispersion with | Dispersion with |
| | uniformity | uniformity | uniformity |
| | with some larger | with some larger | with some larger |
| | white dispersed | white dispersed | white dispersed |
| | particles | particles | particles |
| | (probably CLM) | (probably CLM) | (probably CLM) |

It was found that the order of addition was important to maintain consistency in formulation outcome (i.e., homogeneous blending). The order of addition comprises dissolving LLD in presence of NMP, followed by addition to Kollidon CLM dispersed in Ethyl Acetate. After the dispersion is formed, mixing should be performed. Add other excipients and/or the adhesive are added in final addition steps, followed by mixing to homogenize.

It was also found that NMP is not feasible as an excipient, to remain present in the formulation at a known and controllable concentration, in the presence of heptane due to partial evaporation during drying process to eliminate heptane. Thus, NMP should be treated as a process solvent in this particular composition instead of inclusion as an excipient with unpredictable evaporation at this stage of formulation.

Due to the nature of polarity of the NMP, the formulation focus was shifted to evaluate a silicone-based pressure sensitive adhesive composition comprising drug, with crospovidone, silicone PSA and ethyl acetate as the primary processing solvent as it was already being incorporated into a dispersion of crospovidone in prior formulations. Formulation Table 17 provides the initial formulations using a silicone PSA.

TABLE 17
Solid Drug Dispersion in Adhesive Formulations with Silicone PSA

| | RDNB- | RDNB- | RDNB- | RDNB-0003- |
|---|---|---|---|---|
| Excipients | 0003-7-1 | 0003-7-2 | 0003-7-3 | 7-8 |
| NMP as | 10 × [LLD] | 10 × [LLD] | 10 × [LLD] | 10 × [LLD] |
| process | | | | |
| solvent | | | | |
| LLD | 1.0% | 1.0% | 1.0% | 1.0% |
| Kollidon CLM | 5.0% | 7.5% | 10.0% | 5.0% |
| Oleth-3 | — | — | — | 5.0% |
| BIO-PSA | 94% | 91.5% | 89% | 89% |
| 7-4202 | | | | |
| Ethyl Acetate | 5 × Kollidon | 5 × Kollidon | 5 × Kollidon | 5 × Kollidon |
| as process | CLM | CLM | CLM | CLM |
| solvent | | | | |
| Observations | Dispersion | Dispersion | Dispersion | Dispersion |
| | with | with | with | with |
| | uniformity | uniformity | uniformity | uniformity |
| | with some | with some | with some | with some |
| | larger white | larger white | larger white | larger white |
| | dispersed | dispersed | dispersed | dispersed |
| | particles | particles | particles | particles |
| | (probably | (probably | (probably | (probably |
| | CLM) | CLM) | CLM) | CLM) |

The formulations shown in Table 17above evaluated the concentration of crospovidone and determine relationship between LLD: crospovidone ratio in presence of silicone PSA matrix.

Tests were performed to assess the release of the drug from the formulation and skin permeation of the drug from the matrix through human cadaver skin as the stratum corneum layer.

Quick Dissolution: A test used to assess possible drug release from solution in a small and efficient technique was conducted. Samples of a fixed unit size were placed into a 20 mL vial with media. Assays by HPLC were carried out after 24 hours to assess mg/mL and % release from each patch, with the results summarized below in Table 18.

TABLE 18
Quick Dissolution Test

| Formulations | Drug released (mg/mL) | % Drug released |
|---|---|---|
| RDNB-0003-7-1 | 42.0 | 28.4% |
| RDNB-0003-7-2 | 112.7 | 79.9% |
| RDNB-0003-7-3 | 88.0 | 80.9% |
| RDNB-0003-7-8 | 107.0 | 73.1% |

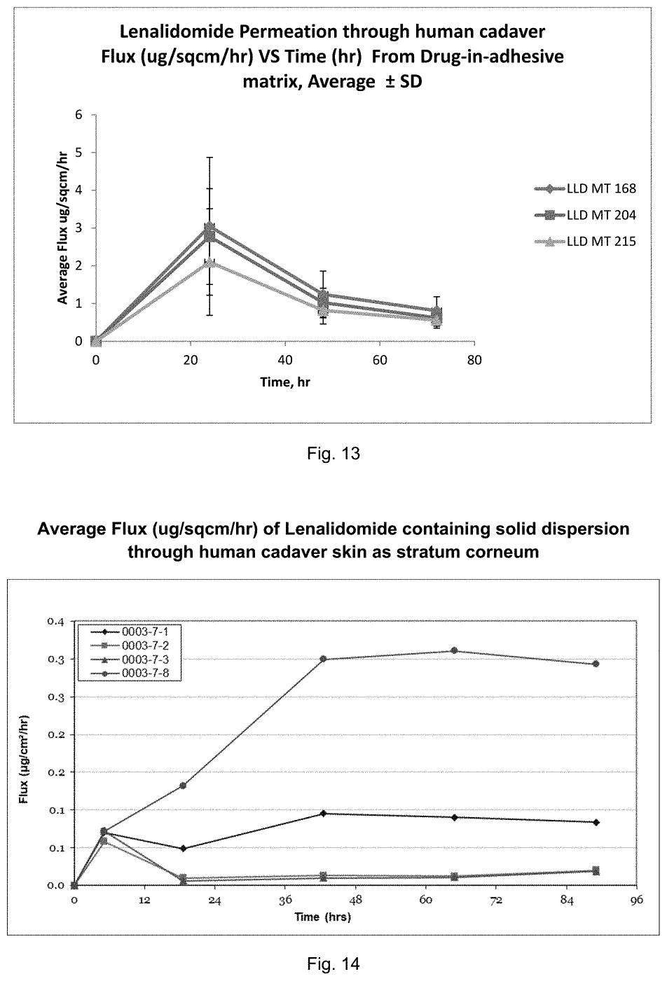

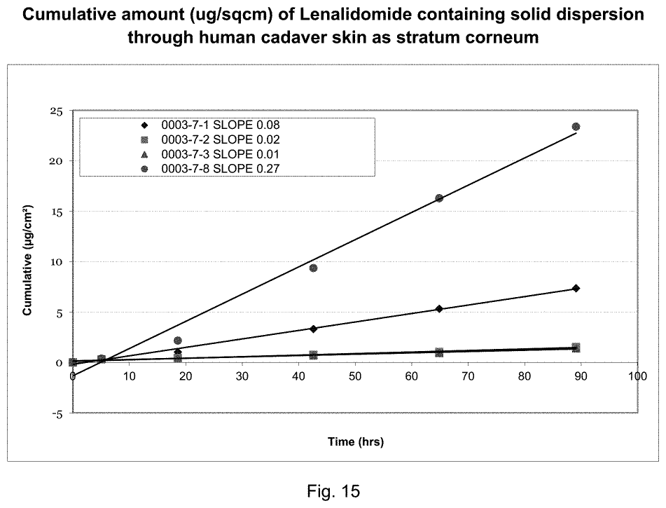

The formulations from Table 18 above contained 1% w/w LLD of dry adhesive composition as a known solubility concern was evident with this API. The flux of these formulations through human cadaver skin is shown in FIGS. 14 and 15.

The above graphs show distinct and definitive increase in flux based on a few changes in the formulation approach. Namely:
1. Flux appears to be significantly increased by incorporation of Oleth-3 within the formulation.
2. Flux appears to be increased with lower ratio of LLD to crospovidone where a 1:5 is higher flux compared to a 1:7.5 and 1:10 ratio.
3. Oleth-3 appears to overcome a barrier of drop in flux after initial 24 hours.
4. Although, not at a significant flux level to achieve and reach a target flux of about 2-5 μg/cm²/hr, the concept of drug delivery of lenalidomide through the skin from a solid dispersion is shown to be possible.

Surprisingly, a high melting point drug with a negative log P has the ability to be incorporated into a solid dispersion matrix which provides ability for skin permeation to be achieved for lenalidomide.

Recent studies have shown zeroes for solid dispersions of drug in matrix pressure sensitive adhesives and crospovidone without dispersing solubilized drug however as solid drug dispersed within the matrix. Thus, conceptually, incorporation of a solubilized drug to create a solid dispersion by precipitating the drug onto or into a substrate such as Kollidon CLM is possible where incorporation of a solid drug into a similar matrix without solubilizing the drug prior to preparation of the solid dispersions does not exhibit skin permeation at a measurable rate.

Hypothetically, it is proposed that the addition of a surfactant into the system allows for better distribution and or solubility of lenalidomide in the presence of a hydration event during diffusion through skin in which water from media enters the adhesive matrix from the skin during hydration under occlusion, thereby releasing the drug, and in this case, a surfactant possibly modifying the solubility of the drug on the skin.

### Example 3

Example 3 focuses on the development of a transdermal drug delivery system with separate adhesive and drug in polymer matrix layers.

Table 19 below shows two initial formulations that were tested for the drug in polymer matrix layer. The results from flux/permeation testing are also shown in Table 19.

TABLE 19
Drug in Polymer Layer Formulations

| Ingredients | P16 (% w/w) | P23 (% w/w) |
|---|---|---|
| Lenalidomide | 3.7 | 3.74 |
| NMP | 16.7 | 16.69 |
| DMSO | 28.8 | 28.77 |
| PEG-400 | 10.7 | 10.71 |
| Lactic acid | 11.1 | 11.08 |
| Methyl laurate | 5.0 | 4.98 |
| Lauryl lactate | 5.0 | 4.98 |
| Brij O10 | 5.0 | 4.98 |
| PVPK-90 | 1.2 | 1.25 |
| HPMC AS MF | 11.6 | 10.34 |
| Aerosil ® | 1.2 | 1.25 |
| Klucel ® HF | — | 1.25 |
| Total | 100.0 | 100.0 |
| No. Of cells | 34 | 17 |
| Avg Flux 0-24 hr, ug/sqcm/hr | 3.53 (71%) | 4.91 (55%) |
| Avg Flux 24-48 hr, ug/sqcm/hr | 2.01 (41%) | 2.53 (45%) |
| Avg Flux 48-72 hr, ug/sqcm/hr | 1.68 (42%) | 2.14 (44%) |
| Avg | 2.40 (51%) | 3.19 (46%) |

### Blend Preparation

The above ingredients (NMP, DMSO, Lactic acid, PEG-400, Methyl Laurate, Lauryl Lactate, Brij® O10 and Aerosil®) were mixed together for 30 minutes. The required amount of PVPK-90 was solubilized in above solution. Upon polymer solubilization, LLD was added followed by stirring for 30 minutes. The remaining excipient HPMCAS-MF and/or Klucel HF was added and the formulation was stirred for 18 hours to solubilize the polymers. After 18 hours of mixing, the blended formulations were sonicated for 30 minutes in order to remove any air bubbles produced due to mixing.

### Coating

The current polymer blend is highly hydrophilic in nature and it required some hydrophilic substrate in order to coat on the backing membrane. Duro-Tak® 9301 was chosen based on it having the lowest solubility for LLD and for its compatibility with the polymer blend. 0.1 mm of Duro-Tak® 9301 was coated onto the release liner Scotchpak® 9744 and dried for 10 minutes at 85° C. following 10 minutes of room temperature drying. The backing membrane was applied to the dried adhesive laminate in order to transfer the adhesive layer to the backing membrane. 0.2 mm of polymer blend is coated on the adhesive layer (for a total thickness of 0.3 mm) and dried at 85° C. for 15 minutes followed by 10 minutes of room temperature drying. The release liner Scotchpak®9744 was applied to the polymer matrix surface. A circular die was used to cut patches (7 sqcm) for subsequent studies. After drying, the drug adhesive matrix had a surface density of 2-30 mg/sqcm, containing LLD in 1-20% w/w.

### In-Vitro Permeation Study

The prepared transdermal formulations were then subjected to a flux measurement test as follows. Human cadaver skin, stored at −80° C., was thawed at room temperature in phosphate buffered saline (PBS), and visually inspected for defects before using in the study. Transdermal flux was then measured using standard Franz diffusion cells composed of a cylindrical donor compartment and a separate water jacketed cylindrical receptor compartment with the volume of 13 mL. The human cadaver skin was clamped between the two compartments with the dermis side facing toward the receptor compartment. After mounting the human cadaver skin on franz diffusion cell, it is pretreated by putting 400 mg/sqcm of gel (PT001) for 1 hr. The receptor compartment was filled with receptor medium, held at constant temperature, and constantly stirred at 600 rpm. After 1 hour, the gel formulation is cleaned from the mounted skin using Kimwipes® and above formulation were applied to the same skin and mounted them on the franz diffusion cells. The receiving media is collected to measure the LLD, as it diffuses through the skin and into receptor compartment. It is important to confirm that the receptor fluid is always in contact with the skin. The receptor compartment was emptied at 24 hour intervals for assay of LLD and replaced with fresh receptor solution. In order to maintain the sink condition in receptor compartment, it is important to keep the LLD concentration in receptor compartment less than 10% of its solubility. The experimental conditions are provided in Table 20 below.

TABLE 20
Experimental Conditions for In Vitro Permeability Testing

| Receiving Media | PBS (pH = 6.0) + 0.01% Sodium Azide |
|---|---|
| Receiving Media Volume (mL) | 13 |
| Sample Volume (mL) | 13 |
| Sampling Interval (hr) | 24, 48,7 2 |
| Franz-cell diffusion area (sqcm) | 1.76 |
| Membrane Type | Human Cadaver Skin |

The flux of LLD through human cadaver skin was measured for a minimum period of 72 hours (3 days) and the results of the flux measurement are provided in the last four rows of Table 19 above.

### Example 4

Example 4 focuses on the development of a pretreatment composition for use with Examples 1-3 above, where application of a gel/spray/solution/wetting agent to the skin prior to application of the drug containing product, intended to be a patch, however it can be another topical dosage form, solution gel, cream, etc. (or as a layer of any of the transdermal drug delivery systems contemplated above) in order to determine if such pretreatment can increase drug permeation.

### Skin Pretreatment and In Vitro Permeability

In vitro permeability from different lenalidomide adhesive matrix patches and lenalidomide polymer matrix patches were conducted with pretreated human cadaver skin.

### Human Cadaver Skin Pretreatment

Human cadaver skin was mounted between donor and receptor compartment of in vitro franz diffusion cells. Receptor compartment was filled with receiving medium. Known amount of pretreatment formulation was loaded in donor compartment for specified duration (generally about 1 hour), after that pretreatment formulation was wiped off or removed with a wipe. Immediately lenalidomide adhesive matrix patch formulation or polymer patch formulation was applied to pretreated wiped skin. Permeation of lenalidomide in receptor compartment was quantified at specific time intervals.

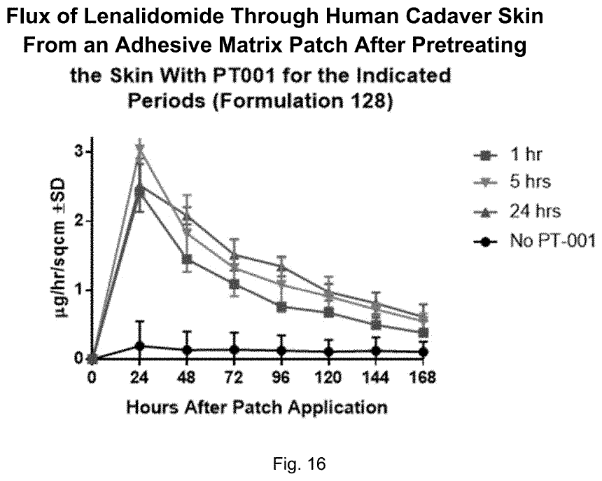

Table 21 and Table 22 show a pretreatment formulation and LLD adhesive matrix patch formulation, where FIG. 16 is a graph showing the improvement in flux upon application of the pretreatment formulation for 1 hour, 5 hours, and 24 hours to human cadaver skin. After 1 hour, 5 hour and 24 hour pretreatment durations, the pretreatment formulation was removed or wiped with wipe from skin. Immediately LLD adhesive matrix formulation was applied to pretreated skin.

TABLE 21
Pretreatment Formulation PT-001

| Ingredients PT-001 | % W/W |
|---|---|
| Dimethyl sulfoxide | 45.7 |
| N-Methyl Pyrrolidone | 26.6 |
| Super refined PEG 400 | 17 |
| Lactic acid (Racemic) | 9.6 |
| Klucel HF | 1.1 |

TABLE 22
LLD Adhesive Matrix Patch Formulation 128

| Ingredients, LLDMT-128 | % W/W |
|---|---|
| Lenalidomide hemihydrate | 2 |
| SR Oleic acid | 16 |
| Isopropyl palmitate | 10 |
| Ethyl cellulose N50 | 10 |
| Kollidon ® 30 LP | 10 |
| Duro-Tak ® 87-2516 | 52 |
| Ethyl Acetate and NMP | Process |
| | solvent |

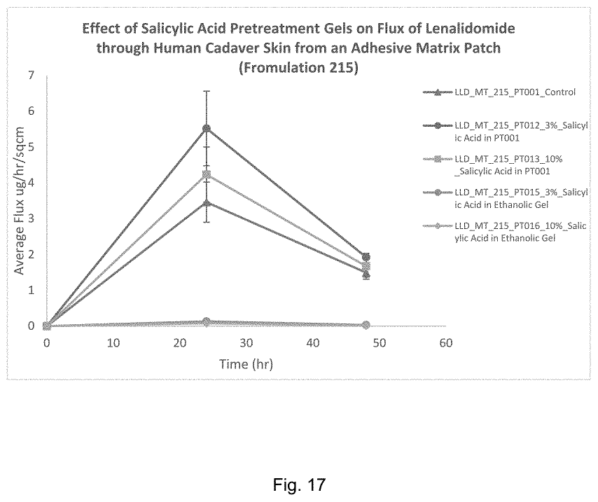

Tables 23-26 show the components of various pretreatment formulations, while Table 27 shows the components of the LLD formulation, where FIG. 17 is a graph showing the improvement in flux upon application of the pretreatment formulation (pretreatment formulation was applied to human cadaver skin for about 1 hour and then it was removed or wiped with wipe from skin. Immediately LLD adhesive matrix formulation was applied to pretreated skin) for up to 72 hours, where pretreatment formulations (PT 012) including DMSO, NMP, PEG 400, lactic acid, salicylic acid, and Klucel HF exhibited improved permeation compared to a control (Pretreatment formulation PT 001) and pretreatment formulation containing only ethanol, salicylic acid, and Klucel HF (Pretreatment formulation PT 015) with improved permeation when the salicylic acid is present at 3 wt. % compared to 10 wt. %.

TABLE 23
Pretreatment Formulation PT 012

| Ingredients PT 012 | % W/W |
|---|---|
| Dimethyl sulfoxide | 45 |
| NMP | 25.9 |
| SR PEG 400 | 16.2 |
| Lactic acid (Racemic) | 8.8 |
| Salicylic acid | 3 |
| Klucel HF | 1.1 |

TABLE 24
Pretreatment Formulation PT 013

| Ingredients PT 013 | % W/W |
|---|---|
| Dimethyl sulfoxide | 43.2 |
| NMP | 24.1 |
| SR PEG 400 | 14.5 |
| Lactic acid (racemic) | 8.8 |
| Salicylic acid | 10 |
| Klucel HF | 1.1 |

TABLE 25
Pretreatment Formulation PT 015

| Ingredients PT 015 | % W/W |
|---|---|
| Ethanol | 96 |
| Salicylic Acid | 3 |
| Klucel HF | 1 |

TABLE 26
Pretreatment Formulation PT 016

| Ingredients PT 016 | % W/W |
|---|---|
| Ethanol | 89 |
| Salicylic Acid | 10 |
| Klucel HF | 1 |

TABLE 27
LLD Adhesive Matrix Patch Formulation 215

| Ingredients, LLDMT-215 | % W/W |
|---|---|
| Lenalidomide hemihydrate | 6.7 |
| SR Oleic acid | 15.3 |
| Ethyl cellulose N50 | 9.5 |
| Kollidon ® 30 LP | 9.5 |
| Duro-Tak ® 87-2516 | 59 |
| Ethyl acetate and NMP | solvent |
| | Process |

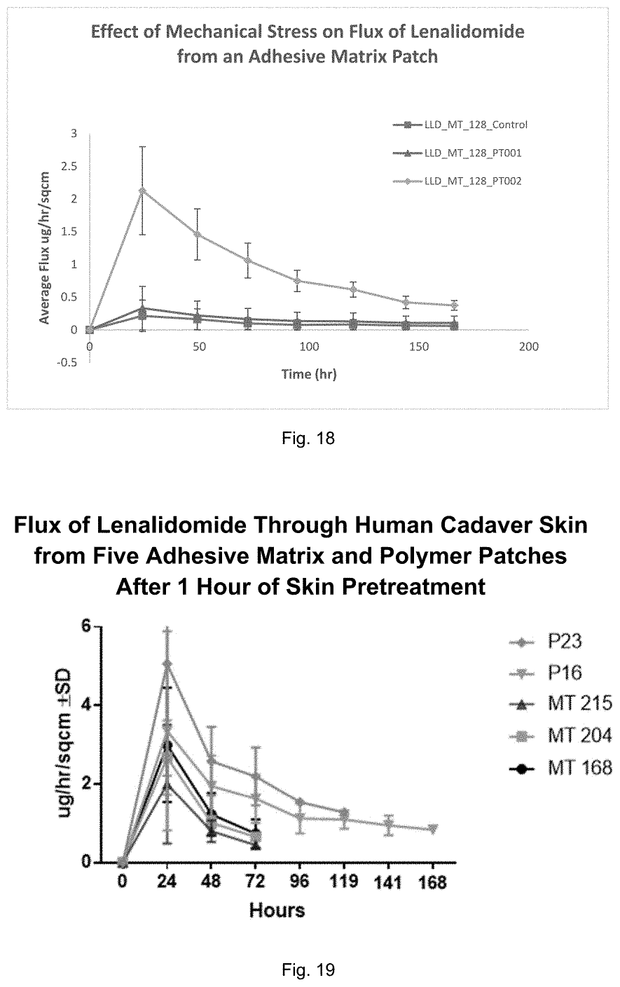

Table 28 shows the components of another LLD adhesive matrix patch formulation, while Tables 29-30 show the components of various pretreatment formulations, and FIG. 18 is a graph showing the improvement in flux upon application of the pretreatment formulation (pretreatment formulation application to skin for about 1 hour. After 1 hour pretreatment formulation was removed or wiped off with a wipe from skin. Immediately matrix formulations were applied to pretreated skin) for up to 168 hours, where the pretreatment formulation including DMSO, NMP, PEG 400, lactic acid, and Klucel HF exhibited improved permeation compared to a control and a pretreatment formulation containing only DI water an Klucel HF.

TABLE 28
LLD Adhesive Matrix Patch Formulation 128

| Ingredients LLD MT-128 | % W/W |
|---|---|
| Lenalidomide hemihydrate | 2 |
| Oleic acid | 16 |
| Isopropyl Palmitate | 10 |
| Ethyl cellulose N50 | 10 |
| Kollidon ® 30 LP | 10 |
| Duro-Tak ® 87-2516 | 52 |

TABLE 29
Pretreatment Formulation PT-001

| Ingredients PT-001 | % W/W |
|---|---|
| Dimethyl sulfoxide | 45.7 |
| N-Methyl Pyrrolidone | 26.6 |
| SR PEG 400 | 17 |
| Lactic acid (racemic) | 9.6 |
| Klucel HF Pharm | 1.1 |

TABLE 30
Pretreatment Formulation PT-002

| Ingredients PT-002 | % W/W |
|---|---|
| DI Water | 98.9 |
| Klucel HF Pharm | 1.1 |

Next, Tables 31 and 32 show the components of 3 drug in adhesive matrix patch formulations (Table 31) and two drug in polymer formulations (Table 32) which were subjected to 1 hour of skin pretreatment with Pretreatment formulation PT-001. The results of the flux of LLD up to about 168 hours are shown in FIG. 19, where it is observed that the peak flux occurred in about 24 hours and the flux was greatest for the two drug in polymer formulations as compared to the three drug in adhesive formulations.

TABLE 31
Drug in Adhesive Matrix Patch Formulations

| Adhesive Matrix Patch | | | |
|---|---|---|---|
| Ingredients (% w/w) | MT 168 | MT 204 | MT 215 |
| Lenalidomide hemihydrate | 3 | 6.7 | 6.7 |
| Oleic acid | 15.8 | 15.3 | 15.3 |
| Isopropyl Palmitate | 9.9 | 4.8 | — |
| Ethyl cellulose N50 | 9.9 | 9.5 | 9.5 |
| Kollidon ® VA 64 | 9.9 | — | — |
| Kollidon ® 30 LP | — | 9.5 | 9.5 |
| Duro-Tak ® 387-2516 | 51.5 | 54.2 | 59 |
| Ethyl Acetate | Process | Process | Process |
| | solvent | solvent | solvent |
| NMP | Process | Process | Process |
| | solvent | solvent | solvent |

TABLE 32
Drug in Polymer Formulations

| Polymer Film | | |
|---|---|---|
| Ingredients % w/w | P16 | P23 |
| LLD | 3.7 | 3.7 |
| NMP | 16.7 | 16.7 |
| DMSO | 28.8 | 28.8 |
| PEG-400 | 10.7 | 10.7 |
| Lactic acid | 11.1 | 11.1 |
| Methyl laurate | 5.0 | 5.0 |
| Lauryl lactate | 5.0 | 5.0 |
| Brij O10 | 5.0 | 5.0 |
| PVPK-90 | 1.3 | 1.3 |
| HPMC AS MF | 11.6 | 10.3 |
| Aerosil ® | 1.3 | 1.3 |
| Klucel ® HF | — | 1.3 |

### Example 5

Example 5 is directed to a nonclinical study with a New Zealand white rabbit animal model. 5 groups were treated with various formulations, with Groups 2-5 discussed herein, as Group 1 was treated with in IV solution of lenalidomide, while Groups 2-5 were treated with various transdermal drug delivery systems (patches) containing lenalidomide. Group 2 was treated with a solid dispersion of a drug in adhesive layer formulation (Formulations A and B, see Tables 33 and 34 below), Group 3 was treated with a solid dispersion of a drug in adhesive layer formulation (Formulations A and B, see Table 33 below), Group 4 was treated with an adhesive matrix patch formulation (Formulations C and D, see Table 34 below), and Group 5 was treated with a polymer film patch (Formulations E and F, see Table 35 below).

TABLE 33
LLD Solid Dispersion of Drug in Adhesive Layer Formulations

| Ingredients % w/w | Formulation A | Formulation B |
|---|---|---|
| Lenalidomide hemihydrate | 2 | 0 |
| Oleth-3 | 20 | 20 |
| Kollidon ® CL-M | 5 | 5 |
| Duro-Tak ® 87-9301 | 73 | 75 |
| Ethyl Acetate and NMP | Process solvent | Process solvent |

TABLE 34
LLD in Adhesive Matrix Formulations

| Ingredients % w/w | Formulation C | Formulation D |
|---|---|---|
| Lenalidomide hemihydrate | 5 | 0 |
| Oleic acid | 16 | 16 |
| Kollidon ® 30 LP | 30 | 30 |
| Duro-Tak ® 87-2516 | 39 | 44 |
| Ethylcellulose N50 | 10 | 10 |
| Ethyl Acetate, NMP, Methanol, | Process solvent | Process solvent |
| Ethanol, Heptane | | |

TABLE 35
Drug in Polymer Film Formulations

| Ingredients % w/w | Formulation E | Formulation F |
|---|---|---|
| Lenalidomide hemihydrate | 2.31 | 0 |
| PEG-400 | 26.01 | 26.6 |
| Lauryl lactate | 11.56 | 11.8 |
| Methyl laurate | 11.56 | 11.8 |
| Brij O10 | 11.56 | 11.8 |
| Aerosil ® 200 Pharma | 2.89 | 3.0 |
| BHT | 1.45 | 1.5 |
| Kollidon ® K-90 | 2.89 | 3.0 |
| Eudragit EPO | 2.89 | 3.0 |
| HPMC AS MF | 26.88 | 27.5 |
| Duro-Tak 87-9301 | Base layer on | Base layer on |
| | backing | backing |
| NMP, DMSO, and Ethyl Acetate | Process solvent | Process solvent |

In the study, there were four transdermal application sites per animal (1 placebo and 3 active patches). Groups 1 and 2 were not exposed to a pretreatment prior to application of the patches, Group 3 was exposed to a 1 hour pretreatment with 1 milliliter of DMSO applied onto non-stick pads, and Groups 4 and 5 were exposed to a 1 hour pretreatment of Pretreatment H. Pretreatment H was prepared by soaking a cotton pad in PT-001 (see Tables 21 and 29 above) applied onto cotton pads. Each cotton pad had an approximate size of 3.5 cm×3.5 cm with approximately 3.8 g of PT-001 applied to each pad. Each pretreatment and patch were secured to the rabbits with a PatchProtect overlay system.

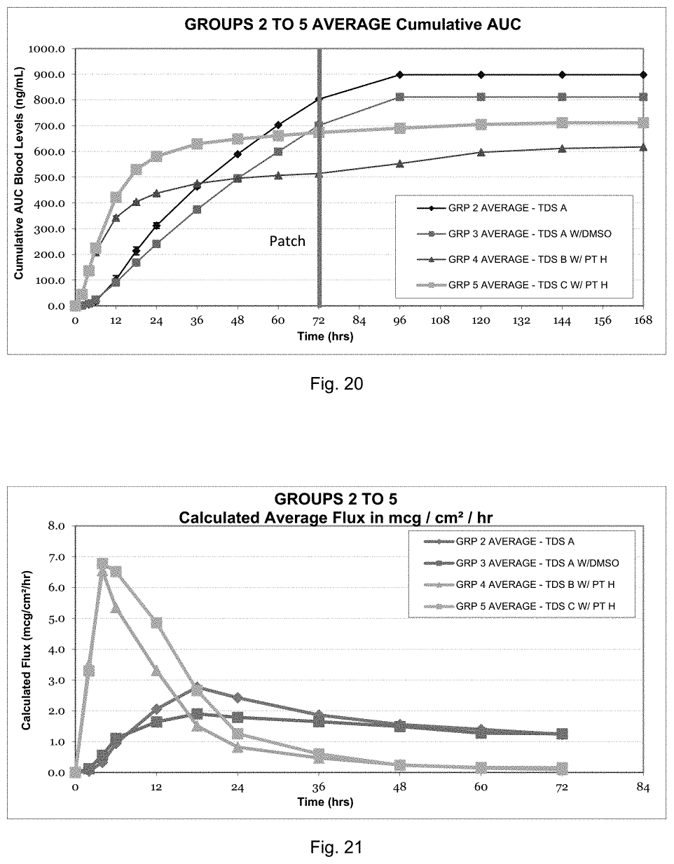

As shown in FIG. 20, the average cumulative area under the curve (AUC) for formulations A-D of lenalidomide delivered via various transdermal drug delivery systems over a time period of 168 hours in a rabbit model. Group 2 was in the form of a solid dispersion of a drug in adhesive layer of Formulation A with no pretreatment; Group 3 was in the form of a solid dispersion of a drug in adhesive layer of Formulation A that was applied after a DMSO pretreatment; Group 4 was in the form of an adhesive matrix of Formulation C that was applied after the PT-001 pretreatment; and Group 5 was in the form of a polymer film of Formulation E that was applied after the PT-001 pretreatment. As can be seen from FIG. 20, both formulated PT-001 pretreatments exhibited a characteristic oral or IV administration delivery profile. Meanwhile, the solid dispersion of the drug in adhesive layer without a pretreatment (Group 2) and the solid dispersion of the drug in adhesive layer with a DMSO pretreatment (Group 3) formulations exhibited a sustained near first-order delivery profile suggesting longer delivery profiles are possible up to 3-days utilizing the transdermal drug delivery systems contemplated by the present invention.

Meanwhile, FIG. 21 is a graph comparing the average flux for the four formulations described in FIG. 20 over a time period of 72 hours, where it can be further seen that the solid dispersion of the drug in adhesive layer lenalidomide transdermal drug delivery systems contemplated by the present invention exhibited sustained delivery compared to an adhesive matrix or polymer film type delivery system.

### Example 6

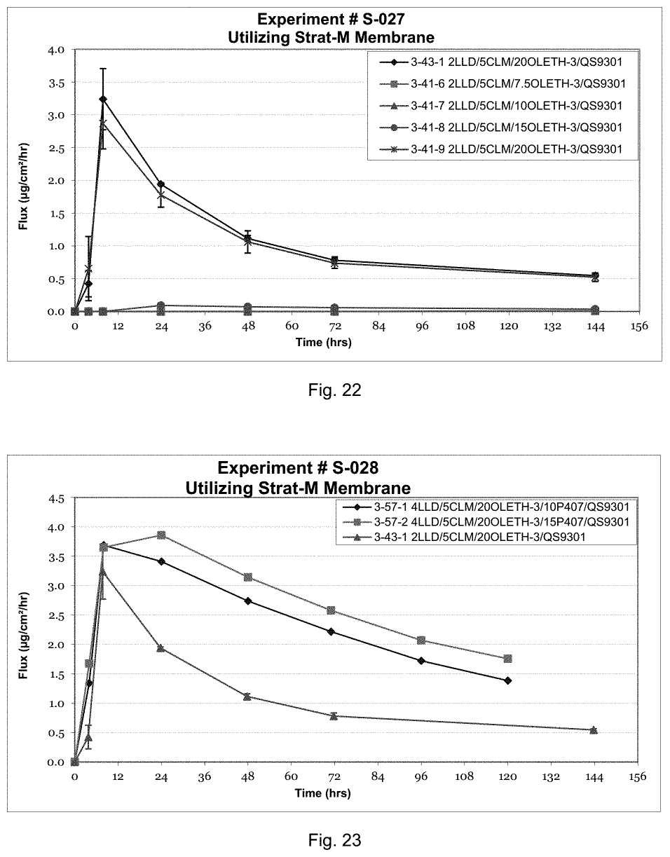

Next, various solid dispersion of a drug in adhesive layer formulations were made that included an oleth-based non-ionic surfactant or a combination of an oleth-based non-ionic surfactant (e.g., oleth-3) and a poloxamer non-ionic surfactant (e.g., P407). The amount of the oleth-based non-ionic surfactant varied from 7.5 wt. % to 20 wt. % (see FIG. 22), while the amount of the poloxamer non-ionic surfactant varied from 0 wt. % to 15 wt. % (see FIG. 23).

As can be seen from FIG. 22, which is a graph showing the permeation of lenalidomide through a Strat-M membrane, as the weight percentage of oleth-3 increased from 7.5 wt. % to 20 wt. %, the solid dispersion of lenalidomide in adhesive layer formulation exhibited improved and sustained delivery for up to about 144 hours.

Meanwhile, FIG. 23 is a graph showing the improved permeation of lenalidomide through a Strat-M membrane that included both an oleth-based non-ionic surfactant and a poloxamer non-ionic surfactant, demonstrating that permeation is improved with an oleth and a poloxamer compared to an oleth alone. As shown, there is a significant increase in AUC due to an increase in the available lenalidomide as well as a permeability improvement, where it is believed that the poloxamer (e.g., P407) improves the solubility of the lenalidomide in the presence of water and the oleth improves the permeation of the available lenalidomide in the solid. This example thus demonstrates that the inclusion of a poloxamer (specifically P407) can lead to significant improvement in the solubility of the lenalidomide in the presence of water and that the oleth can contribute to the permeation of the available lenalidomide.

### Example 7

Next, the effect of different drug concentrations and drying temperature on the flux profile of lenalidomide was evaluated. The various formulations tested are shown below in Table 36. Transdermal flux for each formulation using Franz diffusion cells, where the receptor compartment was filled with receiving medium. Then, human cadaver skin was placed on the cell with the epidermal side facing the donor compartment. The adhesive matrix was then applied to the skin, and the receiving medium was sampled to determine the amount of the drug that diffused through the skin. It should be noted that SD001 was a solid dispersion of a drug in adhesive layer, while the other examples were solubilized drug in adhesive layers.

TABLE 36
LLD Formulations

| | SD001 | PA75 A | PA76 A | PA77 A | PA75 B | PA76 B | PA77 B |
|---|---|---|---|---|---|---|---|
| Excipients | (% w/w) | (% w/w) | (% w/w) | (% w/w) | (% w/w) | (% w/w) | (% w/w) |
| Lenalidomide | 2.00 | 1.6% | 3.1% | 2.9% | 1.6% | 3.1% | 2.9% |
| Oleth 3 | 20.00 | 7.9% | 7.8% | 7.2% | 7.9% | 7.8% | 7.2% |
| Kollidon CL-M | 5.00 | — | — | — | — | — | — |
| Lauryl lactate | — | 7.9% | 7.8% | 7.2% | 7.9% | 7.8% | 7.2% |
| Methyl laurate | | 7.9% | 7.8% | 7.2% | 7.9% | 7.8% | 7.2% |
| Lactic acid | — | — | — | 7.2% | — | — | 7.2% |
| Aerosil | — | 2.0% | 1.9% | 1.8% | 2.0% | 1.9% | 1.8% |
| PVP K-90 | — | 5.1% | 5.0% | 4.6% | 5.1% | 5.0% | 4.6% |
| Durotak 9301 | 73.00 | 67.7% | 67.3% | 61.9% | 67.7% | 67.3% | 61.9% |
| Volatile | NMP, | DMSO | | | | | |
| solvents | Ethyl | | | | | | |
| | acetate | | | | | | |
| Drying | | 60° C., 20 min | | | 110^{o} C., 20 min | | |
| condition | | | | | | | |
| Flux 0-24 Hrs | 0.04 (73) | 0.04 (46) | 0.01 | 0.08 | 0.07 (85) | 0.01 | 0.07 |
| | | | (101) | (120) | | (430) | (150) |
| Flux 24-48 Hrs | 0.10 (59) | 0.11 (29) | 0.06 (56) | 0.13 | 0.13 (55) | 0.05 (35) | 0.16 (96) |
| | | | | (103) | | | |
| Flux 48-72 Hrs | 0.30 (40) | 0.27 (16) | 0.17 (30) | 0.24 (84) | 0.27 (33) | 0.14 (36) | 0.35 (65) |
| Flux 0-72 Hrs | 0.15 (48) | 0.14 (22) | 0.08 (40) | 0.15 (95) | 0.16 (47) | 0.07 (31) | 0.19 (83) |

Further, although the transdermal drug delivery systems described above includes a two layer transdermal formulations which may be in form of a liquid or semi-solid form of a desired degree of viscosity, for example, a polymer film, solution, suspension, nano suspension, micro suspension, dispersion, emulsion, micro emulsion, nano emulsion, gel, ointment, cream, paste, lotion, mousse, or balm. Alternatively, the transdermal formulation may form part of a TDS that comprises the transdermal formulation. Exemplary TDS include, without limitation, topical formulations (e.g. for occlusive or non-occlusive application to the skin or mucous membrane), gels, lotions, sprays, metered dose transdermal sprays, aerosols, suppositories, magma, transdermal patches, bilayer matrix patches, multilayer matrix patches, monolithic matrix patches with or without adhesive, drug-in-adhesive patches, matrix reservoir patches (with a separate matrix reservoir optionally surrounded by adhesive), microreservoir patches, hydrogel matrix patches, mucoadhesive patches, adhesive systems, transdermally applicable tape, microneedle systems, iontophoresis systems, or combinations thereof. In further embodiments, the formulations provided herein provide for stable formulations of the active components in the formulations. For example, the formulations are shelf stable and maintain at least 90% of their activity over a predetermined period, when stored under standard ambient conditions. In further embodiments, the formulations are shelf stable for at least 3 months, 6 months, 9 months, a year, or longer.

Materials to make the transdermal delivery system of the invention in patch form known to those skilled in the art, for example, such as but not limited to reservoir patch, matrix patch, drug in adhesives, transdermal films and may include, such as but are not limited to polymers, copolymers, derivatives, backing film, release membrane, release liners, etc. either alone or in combinations thereof. Pressure sensitive adhesives such as but not limited to silicone polymers, rubber based adhesives, acrylic polymers, acrylic copolymers, polyisobutylene, acrylic acid-isooctyl acrylate copolymer, hot melt adhesives, polybutylene etc.; backing film such as but not limited to ethylene vinyl acetate copolymers, vinyl acetate resins, polyurethane, polyvinyl chloride, metal foils, polyester, aluminized films, polyethylene, etc.; release membrane such as but not limited to microporous polyethylene membrane, microporous polypropylene membrane, rate controlling ethylene vinyl acetate copolymer membrane etc.; release liners such as but not limited to siliconized polyester films, fluoropolymer coated polyester film, polyester film, siliconized polyethylene terephthalate film, etc.; tapes, etc.

Moreover, different techniques and ingredients can be used to increase the stability and/or solubility of drug molecule in formulation such as without any limitation to coating, encapsulation, microencapsulation, nanoencapsulation, lyophilization, chelating agents, complexing agents, etc.

Additionally, each of the transdermal drug delivery systems described in the present invention can include components that act as excipients in addition to or in place of other components of the formulations described above.

### Solvents

The transdermal formulation and/or topical formulation of the invention may comprise solvents known to those skilled in the art either alone or in combinations thereof without any limitation to following like alcohol C₁-C₂₀ such as but not limited to (methanol, ethanol, isopropyl alcohol, butanol, propanol etc.), polyhydric alcohols, glycols such as but not limited to (propylene glycol, polyethylene glycol, dipropylene glycol, hexylene glycol, butyene glycol, glycerine, etc.), derivative of glycols, pyrrolidones such as but not limited to N methyl 2-pyrrolidone, 2-pyrrolidone etc.; sulfoxides such as but not limited to (dimethyl sulfoxide, decymethylsulfoxide, etc.; dimethylisosorbide, mineral oils, vegetable oils, water, polar solvents, semi polar solvents, non-polar solvents, volatile chemicals which can be used to make matrix patch such as but not limited to ethanol, propanol, ethyl acetate, acetone, methanol, dichloromethane, chloroform, toluene, IPA; acids such as but not limited to acetic acid, lactic acid, levulinic acid, bases and others. Such solvents can be present in the formulation in an amount ranging from about 0.01% w/w or w/v to about 95% w/w or w/v.

### Thickeners

The transdermal formulation and/or topical formulation of the invention may comprise gelling agents and/or thickening and/or suspending agents and/or polymers and/or adhesive polymers and/or pressure sensitive adhesive polymers known to those skilled in the art either alone or in combinations thereof without any limitation to following like natural polymers, polysaccharides and its derivatives such as but not limited to agar, alginic acid and derivatives, cassia tora, collagen, gelatin, gellum gum, guar gum, pectin, potassium, or sodium carageenan, tragacanth, xantham, gum copal, chitosan, resin etc.; semisynthetic polymers and its derivatives such as without any limitation to cellulose and its derivatives (methylcellulose, ethyl cellulose, carboxymethyl cellulose, hydroxylpropyl cellulose, hydroxylpropylmethyl cellulose etc.; synthetic polymers and its derivatives such as without any limitation to carboxyvinyl polymers or carbomers (Carbopol® 940, Carbopol® 934, Carbopol® 971p NF), polyethylene, and its copolymers, etc., clays such as but not limited to silicates and bentonite; silicon dioxide, polyvinyl alcohol, acrylic polymers (Eudragit®), acrylic acid esters, polyacrylate copolymers, polyacrylamide, polyvinyl pyrrolidone homopolymer and polyvinyl pyrrolidone copolymers such as but not limited to PVP, Kollidon 30, or poloxamer; isobutylene, ethyl vinyl acetate copolymers, natural rubber, synthetic rubber, pressure sensitive adhesives polymers such as silicone polymers including but not limited to BIO-PSA 4302, BIO-PSA 4202 etc.; acrylate pressure sensitive adhesive polymers such as but not limited to Duro-Tak® 87-2156, Duro-Tak® 387-2287, Duro-Tak® 87-9301, Duro-Tak® 387-2051, etc.; polyisobutylene such as but not limited to polyisobutylene low molecular weight, polyisobutylene medium molecular weight, polyisobutylene 35000 MW, etc.; acrylic copolymers, rubber based adhesives, hot melt adhesives, styrene-butadiene copolymers, bentonite, all water and/or organic solvent swellable polymers, etc. Such thickeners can be present in the formulation in an amount ranging from about 0.1% w/w or w/v to about 90% w/w or w/v.

### Permeation Enhancers

The transdermal formulation and/or topical formulation of the invention may comprise permeation enhancers known to those skilled in the art either alone or in combination thereof without any limitation to the following, such as sulfoxides, and similar chemicals such as but not limited to dimethyl sulfoxide, dimethylacetamide, dimethylformamide, decymethylsulfoxide, dimethylisosorbide, etc.; azone, pyrrolidones such as but not limited to N-methyl-2-pyrrolidone, 2-pyrrolidon etc.; esters, fatty acid esters such as but not limited to propylene glycol monolaurate, butyl ethanoate, ethyl ethanoate, isopropyl myristate, isopropyl palmitate, methyl ethanoate, decyloleate, glycerol monooleate, glycerol monolaurate, lauryl laurate etc.; fatty acids such as but not limited to capric acid, caprylic acid, lauric acid, oleic acid, myristic acid, linoleic acid, stearic acid, palmitic acid etc.; alcohols, fatty alcohols and glycols such as but not limited to oleyl alcohol, nathanol, dodecanol, propylene glycol, glycerol etc.; ethers alcohol such as but not limited to diethylene glycol monoethyl ether; urea, triglycerides such as but not limited to triacetin, polyoxyethylene fatty alcohol ethers, polyoxyethylene fatty acid esters, esters of fatty alcohols, essential oils, hydramol, surfactant type enhancers such as but not limited to Brij®, sodium lauryl sulfate, tween, or polysorbate; terpene, terpenoids and all penetration or permeation enhancers referred in the book “Percutaneous Penetration Enhancers” (Eric W. Smith, Howard I. Maibach, 2005. November, CRC press). Such permeation enhancers can be present in the formulation in an amount ranging from about 0.01% w/w or w/v to about 95% w/w or w/v.

### Plasticizers

The transdermal formulation and/or topical formulation of the invention may comprise plasticizers known to those skilled in the art either alone or in combination thereof without any limitation to following like glycerol and its esters, phosphate esters, glycol derivatives, sugar alcohols, sebacic acid esters, citric acid esters, tartaric acid esters, adipate, phthalic acid esters, triacetin, oleic acid esters and all the plasticizers which can be used in transdermal drug delivery system referred in the book “Handbook of Plasticizers” (George Wypych, 2004, Chem Tec Publishing). Such plasticizers can be present in the formulation in an amount ranging from about 0.01% w/w or w/v to about 95% w/w or w/v.

### Other Components/Excipients

The transdermal formulation and/or topical formulation of the invention may comprise emollients, humectants, skin irritation reducing agents and the similar compounds or chemicals known to those skilled in the art either alone or in combinations thereof without any limitation to following like petrolatum, lanolin, mineral oil, dimethicone, zinc oxide, glycerin, propylene glycol and others. Such components can be present in the formulation in an amount ranging from about 0.01% w/w or w/v to about 95% w/w or w/v.

The transdermal formulation and/or topical formulation of the invention may comprise solubilizers, surfactants, emulsifying agents, dispersing agents and similar compounds or chemicals known to those skilled in the art either alone or in combination thereof without any limitation to following like polysorbate such as but not limited to polysorbate 20, polysorbate 40, polysorbate 60, polysorbate 80, etc.; span such as but not limited to span 80, span 20, etc.; surfactants such as anionic, cationic, non-ionic and amphoteric; propylene glycol monocaprylate type I, propylene glycol monocaprylate type II, propylene glycol dicaprylate, medium chain triglycerides, propylene glycol monolaurate type II, linoleoyl polyoxyl-6 glycerides, oleoyl-polyoxyl-6-glycerides, lauroyl polyoxyl-6-gylcerides, polyglyceryl-3-dioleate, diethylene glycol monoethyl ether, propylene glycol monolaurate type I, polyglyceryl-3-dioleate, caprylocaproylpolyoxyl-8 glycerides, etc.; cyclodextrins and others. Such components can be present in the formulation in an amount ranging from about 0.01% w/w or w/v to about 95% w/w or w/v.

The transdermal formulation and/or topical formulation of the invention may comprise auxiliary pH buffering agents and pH stabilizers and similar compounds known to those skilled in the art which helps to maintain the appropriate pH of formulation preferably in the range of 4.0-8.0 either alone or in combination thereof without any limitation to following such as phosphate buffer, acetate buffer, citrate buffer, etc., acids such as but not limited to carboxylic acids, inorganic acids, sulfonic acids, vinylogous carboxylic acids and others; base such as but not limited to sodium hydroxide, potassium hydroxide, ammonium hydroxide, triethylamine, sodium carbonate, sodium bicarbonate, etc. Such pH adjusters can be present in the formulation in an amount ranging from about 0.01% w/w or w/v to about 30% w/w or w/v.

The transdermal formulation and/or topical formulation of the invention may comprise stabilizers such as but not limited to (sodium metabisulfite, citric acid, ascorbic acid, BHA, BHT), oxidizing agents, stabilizers, discoloring agents, preservatives, moisture scavengers, oxygen scavengers, excipients which retard or prevent hydrolysis, excipients which retard or prevent oxidation and similar compounds or chemicals known to those skilled in the art which promote a stable formulation can be used either alone or in combination thereof without any limitation. Such stabilizers can be present in the formulation in an amount ranging from about 0.01% w/w or w/v to about 50% w/w or w/v.

These and other modifications and variations of the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed is:

1. A transdermal drug delivery system comprising:
a solubilized drug in adhesive layer including an active pharmaceutical ingredient comprising an immunomodulatory agent, a pressure sensitive adhesive, a crystallization inhibitor, and optionally a polar aprotic solvent, wherein the immunomodulatory agent is homogeneously dissolved in the solubilized drug in adhesive layer and is present in an amount ranging from about 0.1 wt. % to about 50 wt. % based on a dry weight of the solubilized drug in adhesive layer; and
wherein the transdermal drug delivery system is a single, double, or multi-layered structure.

2. The transdermal drug delivery system of claim 1, wherein the immunomodulatory agent comprises lenalidomide, pomalidomide, iberdomide or thalidomide.

3. The transdermal drug delivery system of claim 1, wherein the pressure sensitive adhesive comprises an acrylate copolymer, a polyisobutylene, a silicone, or a combination thereof.

4. The transdermal drug delivery system of claim 3, wherein the pressure sensitive adhesive comprises the acrylate copolymer.

5. The transdermal drug delivery system of claim 1, wherein the crystallization inhibitor comprises a polyvinylpyrrolidone.

6. The transdermal drug delivery system of claim 1, further comprising a thickener.

7. The transdermal drug delivery system of claim 6, wherein the thickener comprises cellulose, a cellulose derivative, methylcellulose, ethyl cellulose, carboxymethyl cellulose, hydroxylpropyl cellulose, hydroxylpropylmethyl cellulose, hydroxypropyl methylcellulose, acrylate, an acrylate derivative, or a combination thereof.

8. The transdermal drug delivery system of claim 1, further comprising a skin permeation enhancer.

9. The transdermal drug delivery system of claim 8, wherein the skin permeation enhancer comprises a fatty acid or one of its derivatives, a fatty alcohol or one of its derivatives, a fatty ester or one of its derivatives, a surfactant, a solubilizer, a plasticizer, an emollient, a skin irritation-reducing agent, a buffering agent, or a combination thereof.

10. The transdermal drug delivery system of claim 1, further comprising a skin modifier comprising butylated hydroxytoluene (BHT), butylated hydroxyanisole (BHA), gallic acid, ascorbic acid, ascorbyl palmitate, lactic acid, methyl salicylate, salicylic acid, or a combination thereof.

11. The transdermal drug delivery system of claim 1, comprising the polar aprotic solvent, wherein the polar aprotic solvent comprises n-methyl-2-pyrrolidone (NMP), dimethyl sulfoxide (DMSO), dimethylformamide, dimethyl isosorbide, or a combination thereof.

12. The transdermal drug delivery system of claim 1, further comprising:
a backing layer, wherein the backing layer forms an exterior facing-surface of the transdermal drug delivery system; and a release liner, wherein the release liner is positioned adjacent a skin contacting surface of the solubilized drug in adhesive layer, wherein the solubilized drug in adhesive layer comprises 0.1 wt. % to about 50 wt. % of the transdermal drug delivery system.

13. A transdermal drug delivery system comprising:
a solid dispersion of a drug in adhesive layer including an active pharmaceutical ingredient comprising an immunomodulatory agent, a pressure sensitive adhesive, a crosslinked polyvinylpyrrolidone, and a skin permeation enhancer comprising a surfactant, wherein the immunomodulatory agent is homogeneously dispersed throughout the solid dispersion of the drug in adhesive layer and is present in an amount ranging from about 0.1 wt. % to about 25 wt. % based on a dry weight of the solid dispersion drug in adhesive layer;
wherein the transdermal drug delivery system is a single, double, or multi-layered structure.

14. The transdermal drug delivery system of claim 13, wherein the immunomodulatory agent comprises lenalidomide, pomalidomide, iberdomide or thalidomide.

15. The transdermal drug delivery system of claim 13, wherein the pressure sensitive adhesive comprises an acrylate copolymer, a polyisobutylene, a silicone, or a combination thereof.

16. The transdermal drug delivery system of claim 13, wherein the crosslinked polyvinylpyrrolidone is present in the solid dispersion drug in adhesive layer in an amount ranging from about 0.1 wt. % to about 40 wt. % based on the dry weight of the solid dispersion drug in adhesive.

17. The transdermal drug delivery system of claim 13, wherein a ratio of the immunomodulatory agent to the crosslinked polyvinylpyrrolidone is from about 1:10 to about 4:1.

18. The transdermal drug delivery system of claim 13, wherein the skin permeation enhancer further comprises a fatty acid or one of its derivatives, a fatty alcohol or one of its derivatives, a fatty ester or one of its derivatives, a solubilizer, a plasticizer, an emollient, a skin irritation-reducing agent, a buffering agent, an antioxidant a preservative, or a combination thereof.

19. The transdermal drug delivery system of claim 13, wherein the surfactant comprises a non-ionic surfactant.

20. The transdermal drug delivery system of claim 19, wherein the surfactant comprises a polyoxyethylene or polyethylene glycol ether of a fatty derivative which comprises an oleic acid or oleyl alcohol derivative, a lauric acid or lauryl alcohol derivative, cetyl or ceteryl alcohol, stearic acid or stearyl alcohol or similar fatty derivative of polyoxyethylene, a poloxamer, or a combination thereof.

21. The transdermal drug delivery system of claim 13, further comprising:
a backing layer, wherein the backing layer forms an exterior facing-surface of the transdermal drug delivery system; and a release liner, wherein the release liner is positioned adjacent a skin contacting surface of the solid dispersion drug in adhesive layer, wherein the solid dispersion drug in adhesive layer comprises 0.1 wt. % to about 50 wt. % of the transdermal drug delivery system.
